SEQUENCE LISTING (101 sequences)

SEQ ID NO: 1 - protein, 433 aa, human cytomegalovirus
  ref <308>: DATABASE ACCESSION NUMBER: GenBank/AAS48949
  ref <309>: DATABASE ENTRY DATE: 2016-06-10
  ref <313>: RELEVANT RESIDUES IN SEQ ID NO: (1)..(433)
MDRKTRLSEPPTLALRLKPYKTAIQQLRSVIRALKENTTVTFLPTPSLILQTVRSHCVSKITFNSSCLYITDKSFQPKTINNSTPLLGNFMYLTSSKDLTKFYVQDISDLSAKISMCAPDFNMEFSSACVHGQDIVRESENSAVHVDLDFGVVADLLKWIGPHTRVKRNVKKAPCPTGTVQILVHAGPPAIKFILTNGSELEFTANNRVSFHGVKNMRINVQLKNFYQTLLNCAVTKLPCTLRIVTEHDTLLYVASRNGLFAVENFLTEEPFQRGDPFDKNYVGNSGKSRGGGGGSGSLSSLANAGGLHDDGPGLDNDIMNEPMGLGGLGGGGGGGGKKHDRGGGGGSGTRKMSSGGGGGDHDHGLSSKEKYEQHKITSYLTSKGGSGGGGGGGGGGLDRNSGNYFNDAKEESDSEDSVTFEFVPNTKKQKCG

SEQ ID NO: 2 - protein, 491 aa, human cytomegalovirus
  ref <308>: DATABASE ACCESSION NUMBER: GenBank/ YP_081562
  ref <309>: DATABASE ENTRY DATE: 2018-08-13
  ref <313>: RELEVANT RESIDUES IN SEQ ID NO: (1)..(491)
  ref <308>: DATABASE ACCESSION NUMBER: GenBank/YP_081562
  ref <309>: DATABASE ENTRY DATE: 2018-08-13
  ref <313>: RELEVANT RESIDUES IN SEQ ID NO: (1)..(491)
MESSAKRKMDPDNPDEGPSSKVPRPETPVTKATTFLQTMLRKEVNSQLSLGDPLFPELAEESLKTFERVTEDCNENPEKDVLAELVKQIKVRVDMVRHRIKEHMLKKYTQTEEKFTGAFNMMGGCLQNALDILDKVHEPFEEMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMAYAQKIFKILDEERDKVLTHIDHIFMDILTTCVETMCNEYKVTSDACMMTMYGGISLLSEFCRVLSCYVLEETSVMLAKRPLITKPEVISVMKRRIEEICMKVFAQYILGADPLRVCSPSVDDLRAIAEESDEEEAIVAYTLATRGASSSDSLVSPPESPVPATIPLSSVIVAENSDQEESEQSDEEEEEGAQEEREDTVSVKSEPVSEIEEVAPEEEEDGAEEPTASGGKSTHPMVTRSKADQ

SEQ ID NO: 3 - protein, 580 aa, human cytomegalovirus
  ref <308>: DATABASE ACCESSION NUMBER: GenBank/P19893
  ref <309>: DATABASE ENTRY DATE: 2020-12-02
  ref <313>: RELEVANT RESIDUES IN SEQ ID NO: (1)..(580)
MESSAKRKMDPDNPDEGPSSKVPRPETPVTKATTFLQTMLRKEVNSQLSLGDPLFPELAEESLKTFEQVTEDCNENPEKDVLAELGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEHTMPVTHPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 4 - protein, 1046 aa, human cytomegalovirus
  ref <308>: DATABASE ACCESSION NUMBER: GenBank/AGT36561
  ref <309>: DATABASE ENTRY DATE: 2016-12-01
  ref <313>: RELEVANT RESIDUES IN SEQ ID NO: (1)..(1046)
MSLQFIGLQRRDVVALVNFLRHLTQKPDVDLEAHPKILKKCGEKRLHRRTVLFNELMLWLGYYRELRFHNPDLSSVLEEFEVRCAAVARRGYTYPFGDRGKARDHLAVLDRTEFDTDVRHDAEIVERALVSAVILAKMSVRETLVTAIGQTEPIAFVHLKDTEVQRIEENLEGVRRNMFCVKPLDLNLDRHANTALVNAVNKLVYTGRLIMNVRRSWEELERKCLARIQERCKLLVKELRMCLSFDSNYCRNILKHAVENGDSADTLLELLIEDFDIYVDSFPQSAHTFLGARPPSLEFDDDANLLSLGGGSAFSSVPKKHVPTQPLDGWSWIASPWKGHKPFRFEAHGSLAPAADAHAARSAAVGYYDEEEKRRERQKRVDDEVVQREKQQLKAWEERQQNLQQRQQQPPPPTRKPGASRRLFGSSADEDDDDDDDEKNIFTPIKKPGTSGKGAASGNGVSSIFSGMLSSGSQKPTSGPLNIPQQQQRHAAFSLVSPQVTKASPGRVRRDSAWDVRPLTETRGDLFSGDEDSDSSDGYPPNRQDPRFTDTPVDITDTETSAKPPVTTAYKFEQPTLTFGAGVNVPAGAGAAILTPTPVNPSTAPAPAPTPTFAGTQTPVNGNSPWAPTAPLPGDMNPANWPRERAWALKNPHLAYNPFRMPTTSTTSQNNVSTTPRRPSTPRAAVTQTASQNAADEVWALRDQTAESPVEDSEEEDDDSSDTGSVVSLGHTTPSSDYNDVISPPSQTPEQSTPSRIRKAKLSSPMTTTSTSQKPVLGKRVATPHASARAQTVTSTPVQGRVEKQVSGTPSTVPATLLQPQPASSKTTSSRNVTSGARTSSASARQPSASASVLSPTEDDVVSPVTSPLSMLSSASPSPAKSAPPSPVKGRGSRVGVPSLKPTLGGKAVVGRPPSVPVSGSAPGRLSGTSRAASTTPTYPAVTTVYPPSSTAKSSVSNAPPVASPSILKPGASAALQSRRSTGTAAVGSPVKSTTGMKTVAFDLSSPQKSGTGPQPGSAGMGGAKTPSDAVQNILQKIEKIKNTEE

SEQ ID NO: 5 - protein, 105 aa, human cytomegalovirus
  ref <308>: DATABASE ACCESSION NUMBER: GenBank/AHH35121
  ref <309>: DATABASE ENTRY DATE: 2019-05-07
  ref <313>: RELEVANT RESIDUES IN SEQ ID NO: (1)..(105)
MARRLWILSLLAVTLTVALAAPSQKSKRSVTVKQPSTSTNSDGNTTRNQDVTLSQGGSTTDGNEDYSGEEYDVLITDGDGSEHQQPQEKTDKHKEEHTKENEKTQ

SEQ ID NO: 6 - protein, 222 aa, human cytomegalovirus
  ref <308>: DATABASE ACCESSION NUMBER: GenBank/AAS48924
  ref <309>: DATABASE ENTRY DATE: 2016-06-10
  ref <313>: RELEVANT RESIDUES IN SEQ ID NO: (1)..(222)
MASDVSSHLLTVTQSRWTIHHMYNKLLILALFTPVILESIIYVSGPQGGNVTLVSNFTSNISARWFRWDGNDSHLICFYKRGEGLSTPYVGLSLSCAANQITIFNLTLNDSGRYGAEGFTRSGENETFLWYNLTVKPKPLETTTASNVTTIVTTTPTVIGTKSNVTGNASLAPQLRAVAGFLNQTPRENNTHLALVGVIVFIALIVVCIMGWWKLLCSKPKL

SEQ ID NO: 7 - protein, 171 aa, human cytomegalovirus
  ref <308>: DATABASE ACCESSION NUMBER: GenBank/ADE62343
  ref <309>: DATABASE ENTRY DATE: 2020-04-12
  ref <313>: RELEVANT RESIDUES IN SEQ ID NO: (1)..(171)
MSPKNLTPFLTALWLLLDHSRVPRVRAEECCEFINVNHPPERCYDFKMCNRFTVALRCPDGEVCYSPEKTAEIRGIVTTMTHSLTRQVVHNKLTSCNYNPLYLEADGRIRCGKVNDKAQYLLGAAGSVPYRWINLEYDKITRIVGLNQYLESVKKHKRLDVCRAKMGYMLQ

SEQ ID NO: 8 - protein, 120 aa, human cytomegalovirus
  ref <308>: DATABASE ACCESSION NUMBER: GenBank/F5HBX1
  ref <309>: DATABASE ENTRY DATE: 2011-06-28
  ref <313>: RELEVANT RESIDUES IN SEQ ID NO: (1)..(120)
  ref <308>: DATABASE ACCESSION NUMBER: Uniprot.org/F5HBX1
  ref <309>: DATABASE ENTRY DATE: 2011-06-28
  ref <313>: RELEVANT RESIDUES IN SEQ ID NO: (1)..(120)
MRLIFGALIISLTYMYYYEVHGTELRCKCLDGKKLPPKTIMLGNFWFHRESGGPRCNNNEYFLYLGGGKKHGPGVCLSPHHPFSKWLDKRNDNRWYNVNVTRQPERGPGKITVTLVGLKE

SEQ ID NO: 9 - protein, 159 aa, human cytomegalovirus
  ref <308>: DATABASE ACCESSION NUMBER: GenBank/F5HA06
  ref <309>: DATABASE ENTRY DATE: 2011-06-28
  ref <313>: RELEVANT RESIDUES IN SEQ ID NO: (1)..(159)
  ref <308>: DATABASE ACCESSION NUMBER: Uniprot.org/F5HA06
  ref <309>: DATABASE ENTRY DATE: 2011-06-28
  ref <313>: RELEVANT RESIDUES IN SEQ ID NO: (1)..(159)
MLLTWLHHPILNSRIKLLSVRYLSLTAYMLLAICPIAVRLLELEDYDKRCRCNNQILLNTLPVGTELLKPIAASESCNRQEVLAILKDKGTKCLNPNAQAVRRHINRLFFRLVLDEEQRIYDVVSTNIEFGAWPVPTAYKAFLWKYAKKLNYHYFRLRW

SEQ ID NO: 10 - protein, 175 aa, human cytomegalovirus
  ref <308>: DATABASE ACCESSION NUMBER: GenBank/P17150
  ref <309>: DATABASE ENTRY DATE: 2005-02-15
  ref <313>: RELEVANT RESIDUES IN SEQ ID NO: (1)..(175)
  ref <308>: DATABASE ACCESSION NUMBER: Uniprot/P17150
  ref <309>: DATABASE ENTRY DATE: 2005-02-15
  ref <313>: RELEVANT RESIDUES IN SEQ ID NO: (1)..(175)
MLSVMVSSSLVLIVFFLGASEEAKPATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 11 - protein, 139 aa, human cytomegalovirus
  ref <308>: DATABASE ACCESSION NUMBER: GenBank/P17150
  ref <309>: DATABASE ENTRY DATE: 2005-02-15
  ref <313>: RELEVANT RESIDUES IN SEQ ID NO: (1)..(139)
  ref <308>: DATABASE ACCESSION NUMBER: Uniprot.org/P17150
  ref <309>: DATABASE ENTRY DATE: 2005-02-15
  ref <313>: RELEVANT RESIDUES IN SEQ ID NO: (1)..(139)
  ref <301>: AUTHORS: Vivian P. Young
  ref <302>: TITLE: Detection of HCMV viral IL-10 ((vIL-10) in Healthy Blood Donors
  ref <306>: PAGES: 18
  ref <307>: DATE: 2015-05-17
  ref <313>: RELEVANT RESIDUES IN SEQ ID NO: (127)..(139)
MLSVMVSSSLVLIVFFLGASEEAKPATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCVSVSVAALSAQR

SEQ ID NO: 12 - protein, 576 aa, human cytomegalovirus
  ref <308>: DATABASE ACCESSION NUMBER: uniprot.org/P09722
  ref <309>: DATABASE ENTRY DATE: 2005-02-15
  ref <313>: RELEVANT RESIDUES IN SEQ ID NO: (1)..(576)
  ref <308>: DATABASE ACCESSION NUMBER: Uniprot.org/P09722
  ref <309>: DATABASE ENTRY DATE: 2005-02-15
  ref <313>: RELEVANT RESIDUES IN SEQ ID NO: (1)..(576)
MSLLTKAAAEAWGTYLRQRDERCEDAIRCDYGVFQFRNTVFQKTLSMLQGLYLRQYDPPALRTYVQRHQGTTVALRNPANWFLVMREQAAIPQIYARSLAADYLCCDDTLEAVGVLAVRPPDSDLTRNTKQAQELPCVLMLSHYGTVYVYDWETDGLYEVASDIKAFSKNGLLWCEYVYRHPQTPFATTEPRYHVQKFLCTDPTDAAAVAKTAREMSGLNLVIRTPGRTEVEPLLMLGSIEGLRACRPFDHMPAADFRDLLNFIRQRLCCEWYVVGLVGYYLAYGPFVPSGLVLLDKFGVVYLHKIEDSDLYRIADNFHMFLKCGLLKLRGLCRFDRGLRGECRLEELPVCHHTLKRDVLRWHGALGTITRSQLESALDWFLRPTRGTDKVPNNSSAWGRTDLLPTGALQDNQNWAFPSHSVETLPALQGGLWEDNDETTQTVDGQRCFRMPKFFPPPMCRVPYNECGVELPGGDSSDEDESGRPRRIANRIGDTPETPCNSENEDDTTVEGTSGGPEAMDSQAPYPSEDSPTTEKEAWLQRGRRAKAMHARGHTLKQVPIPEPDQMGDDGPDPGP

SEQ ID NO: 13 - protein, 163 aa, human cytomegalovirus
  ref <308>: DATABASE ACCESSION NUMBER: Uniprot.org/F5HEF3
  ref <309>: DATABASE ENTRY DATE: 2011-06-28
  ref <313>: RELEVANT RESIDUES IN SEQ ID NO: (1)..(163)
MNLEQLINVLGLLVWIAARAVSRVGPHGSGLVYRELHDFYGYLQLDLLGPVVAGNRSVRTWREQADRARGTFVRRSGLNTSHILPVGGLSGGSGTLPAGLYRPEEEVFLLLNRCHGPLSTPKSACLAEVGVANASFLSRFNVGDFHGASWENGTAPDGEPGVC

SEQ ID NO: 14 - protein, 473 aa, human cytomegalovirus
  ref <308>: DATABASE ACCESSION NUMBER: Uniprot.org/Q6SWB8
  ref <309>: DATABASE ENTRY DATE: 2004-07-05
  ref <313>: RELEVANT RESIDUES IN SEQ ID NO: (1)..(473)
MLWAHCGRFLRYHLLPLLLCRLPFLLLFQRPQWAHGLDIVEEDEWLREIQGATYQLSIVRQAMQHAGFQVRAASVMTRRNAVDLDRPPLWSGSLPHLPVYDVRSPRPLRPPSSQHHAVSPELPSRDGIRWQYQELQYLVEEQRRRNQSRNAIPRPSFPPPDPPSQPAEDARDADAERTESPHSAESTVRHDASENAVRRRHERRRYNALTVRSRDSLLLTRIRFSNQRCFGRGRLRHPAGSGPNTGGPRPGGAGLRQLRQQLTVRWQLFRLRCHGWTQQVSSQIRTRWEESNVVSQTATRVRTWFVERTTFWRRTWVPRQNPAAEAQELAVIPPAPTVLRQNEEPRQQLTGEETRNSTHTQREEVEDVSREGAREGNDGSRASGNDERRNNAGRYDDDHEVQEPQVTYPAGQGELNRRSQEENEEGGPCESPPMTTNTLTVACPPREPPHRALFRLCLGLWVSSYLVRRPMTI

SEQ ID NO: 15 - protein, 869 aa, human herpesvirus 6A
  ref <308>: DATABASE ACCESSION NUMBER: Uniprot.org/A0A219XZB7
  ref <309>: DATABASE ENTRY DATE: 2017-10-25
  ref <313>: RELEVANT RESIDUES IN SEQ ID NO: (1)..(869)
MDLQRHPIPFAWLDRDKVELLTNFLSNLGRLENVDLREHPHVTNSCVVREGEDVDDLKTLYNLLVLWLMYHYVLSKMKPDYNTIWQDITKLQSVVNEYLKSKGLNKGIFENMFTNKEKFESQFSDINRALLRLGNSIKWGSNVAIHTPYVNLTAEDSSEIENNLQDAEKNMLWYTVYNINDPWDENGFLVTSINKLIYLGKLFLALKQSWSKLEKVAMSQIVITQNHLSGHLSRNDNFNIVYSHRVLQTPLTGQRVESFLKIITSDYDIIKSSLESHSASKAFSVSEIGPHSLMDFVSLDGKIPSNLTLPSMSIDTKKSSVDPARLKKSESRSFDTFLMQRQPKFLELDSVDNAGGKILIKEATLGGEDVKATTPASSVSLMSGVESPSSFTSTNLDLPLSSFTSTNLDLRDKSHGNYKIGPSGILDFNVKFPPDAQLNTNDVDLLQDKTSIGSPSSGITDVVNGFANLNLHQNKSNVSPPWSRNTAANADFLDPVHRFVPEQTGTPFVLNNSDVAGSEENHTTYSTETGVSPRNVFLIKDLRGKDGFRKQKQSDIPKSLTKERNDKAIMHSREVTGDSGDATETVGARNSPALRKIKQANDFFAGLNKKNDRDVLRGGKGNSKDLHSGGNAKKKEMSGKFNVDKEMTRNGQEPSRSLMGDARNAGDGQYIQAGLGQRVNNLLSQFTNLISLGEKGIEDILQNQRGTELKLATENKSGRESEEANVKRILEVSNPQDMFKNFRLQNNLDSVQSPFRLPDVDLSRELDSASFKDALDLKLPGNGEREIDLALEKVKVGETETSDLKVGQDESFVPAQLMKVETPEEKDDVIERMVLRIRQDGETDENTVSGPGVAESLDIEAKGESAIAS

SEQ ID NO: 16 - protein, 755 aa, human herpesvirus 7
  ref <308>: DATABASE ACCESSION NUMBER: Uniprot.org/P52519
  ref <309>: DATABASE ENTRY DATE: 1996-10-01
  ref <313>: RELEVANT RESIDUES IN SEQ ID NO: (1)..(755)
MKMSHLPFAWISDEAKCFLSRFFENISSLPVVDIRENPWILSQCIVKTGNSINNVKTLYNNLILWIYFHQTLCKKKPDYEEVWQEILKVQKILKDYLEQRQMITDYSSLTSFNKVGFETEFKNVAKDLLKLGSFLRWGTVTHAADYVNLTTEERAEIGENLQKAKNNMLSFTIYQIVDPWNENGYYVTNINRLLYLGNLLITLHGSWMNMEKLALNTINEKKNAILKAIENNKNFVSIYSYQILSLPLTSHRVTSFFKILTEDFDVITKSLELHALPVKSTWDDRVKFTPEPIQTFKVLTDLSKSSLSNQFESSSKKTSHGSSFNPEPFIKTEQRSNNTLSKDLFVGSEDGLLSSVKKDSMILDEPRNSTSINNSKKMHRILQTEILDLTDQTMHRPEDKVNQFNEIAVAPDGINQVIDTLSKLDLHNSNKVIDIVSSPKVNVVQLPKNKIDYHSTFFLPENEVNRQNGVQSRDQLSKNSTNDLQKILELRERIKTIKQNNEDIFKLPSEKRRKEIVHENLQSFDDEHNEMSLPPQDQKSIKQKNGNKANSSTKTLNMIGTNDVNASMKEKESASSAKKNQLVKDVKWTPSSSLLDLSRRNDLLQKELFESGLGEKVKKLLTDFTDTISLEERSLKDVLEPPKKTDVSNIATFNDNNLKNLLNSRKRDPLFQNFSFTEKMQPVRSPFFLPNAEIQDFDSGSLLTGKETQNTIFGASKAQENGDKDLIDLENSVQKDDDIVNKLVSHLTHSEEDVV

SEQ ID NO: 17 - protein, 907 aa, Epstein-Barr Virus
  ref <308>: DATABASE ACCESSION NUMBER: Uniprot.org/P03200
  ref <309>: DATABASE ENTRY DATE: 1986-07-21
  ref <313>: RELEVANT RESIDUES IN SEQ ID NO: (1)..(907)
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATSPTLGKTSPTSAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 18 - protein, 941 aa, Human herpesvirus 6A
  ref <308>: DATABASE ACCESSION NUMBER: Uniprot.org/Q69567
  ref <309>: DATABASE ENTRY DATE: 2008-07-22
  ref <313>: RELEVANT RESIDUES IN SEQ ID NO: (1)..(941)
MEPAKPSGNNMGSNDERMQDYRPDPMMEESIKEILEESLMCDTSFDDLIIPGLESFGLIIPESSNNIESNNVEEGSDGELKTLAAQSAGNCIQSIGASVKAAMKQEQSDMEDKLIKCAGLLTQQQSMFIGLGLEQLSQLININLLSSASTKYVESYSKMLHGKELDFFNWCEPRFIVFACDKFDGLVKKVASESRNLLLDLRANMNNDIIKAVKDIFSKATVTLDCQKLNQGATMLMMMAHNKEMSNPDISSKDFCEKINTLKQTLLEGKNEIVETNAKNMQILQTFAIKQMNQIFMDGCDKAFLKLNVNCKNLITAAKNLANTILQSIVICSNEFSWQHLKLLRRGFKVTMLNMITQACECLESDYDDTGLIKPLTPLQIMDGYINMNKNRQSSICDGNTDPSDSMILDLADFDDHGRYSEESSIESIHEDDDNKMYPCTPSPEVPGKSKYVGTFTENSRQSGDEQTNPNCVGTASVTDLGGPDNLNSISGLQSCKNMLLERLLDTQCDSVVEGTEQDGSYGNTLISEMMMFGYETDHSAPYESESDNNDEIDYIANSDSAARTNNIHMNSTDENTPFSNSICSPPEVTPSKKNVKPKSMTPGSKPKKRVAKRKHVSSKSPKNKKIKTDQLPKAADVIVISSESEDEEDGDNIIGNSILIKAIKSESDSESSSESNDCTSEHKQLHLSDYDEVTNNGHCPSYGFPTPVFTIPIRSMQGTGGIKSKFIPKKNWIWYMKKTHQVDNCPIHNSENVDAKDDSDGTEAKHCFMNHFVPIKTDDEDYDKNNVSYIYNKIQNSKIDSGDIIPTKKLIIDMVMDNFMDLNDIIKQGITKHCQDLCNKYNVVTPTTCEDDLNMTNSQTFATTATQVFDPPVTGNNSSILNIINDTTSQNDENRCTEGTSNSNEKCTNISDCNSDGTEAFKLDGYPSDYDPFVENAQIY

SEQ ID NO: 19 - protein, 1500 aa, human herpesvirus 6A
  ref <308>: DATABASE ACCESSION NUMBER: Uniprot.org/Q77Z83
  ref <309>: DATABASE ENTRY DATE: 2008-07-22
  ref <313>: RELEVANT RESIDUES IN SEQ ID NO: (1)..(1500)
MEPAKPSGNNMGSNDERMQDYRPDPMMEESIKEILEESLMCDTSFDDLIIPGLESFGLIIPESSNNIESNNVEEGSDGELKTLAEQKCKQGNDNDVIQSAMKLSGLYCDADITHTQPLSDNTHQDPIYSQESRIFTKTIQDPRIVAQTHRQCTSSASNLQSNESGSTQVRFASELPNQLLQPMYTSHNQNANLQNNFTSLPYQPYHDPYRDIESSYRESRNTNRGYDYNFRHHPYRPRGGNGKYNYYNPNSKYQQPYKRCFTRTYNRRGRGHRSYDCSDRSADLPYEHYTYPNYEQQNPDPRMNNYKDFTQLTNKFNFESYDYSMAFSTDSTHVQSDNYNHPTKAQTIPETTKTKKHEATKDNETSTENQVLTPDVISLSYRPSSYKMDIIKKIYDTDVIPLPKEALTANGSNRDVDIQKYKKAHIRCRSVQKKKERSSQTNKHDENHASSRSDLKERKSNENEDKAVTKARDFSKLNPLLSPLPLTPEPAIDFADHTDKFYSTPEFNQIQQNLHRSKTSLQDTVPISKHTPRAPTKDNSYKKHHDSKDNYPKMKHSPGRTTSKKNTTNSNGHQNFKEVSVKNVSGKATSTSPKSKTHHYSSSSDEEGQYKSPVKTIIQSPSPYCKLKNPSIMDKNSAKNHTASADKNLTDNSPIRSNLNPTAFNKSNSNKSITDSTSNSDECTDRKPNCNSTKNESKDPNRTCGKNSDKHLSKSCTMASKRAPSRASSRASSRDSSRASSRASSRASSRDSSRASSRASSRDSSRASSRASSRASSKASSRASSRASSRASSRDSSRASSKASSRASSRDSSRASSRASSRASSKASSRASSRASSRASSRDSSRASSKASSRASSRDSSRASSRDSSRDSSRASSRASSRDSSRASSKASRKASSRASSRASSRASSKASGKASSEASSRASSRNSSRASSRASSRASSRDSSRASSRASSRDSSRASSKASRKASSRASSRASSRELRQIYCDSNKRQTPPHDTSINTKFEISEIKFRCGEDLNFYKNTAARLQCFNHNDQFYNPRFRPHIRTNRKKSESTNDTDSESSMSRCKSHCRNSPDSLTVVRRKKHKSGSSSISSSIEENCRSNSHIVTGKEKFTPFYYQSSRTRSSSSSSSSSASLSCSKSTLKTCRKTQYKDNKQIKSKSDSKHKTTNMSSDYESNRHADVFKNSPEAGEKFPLHNSSPFNTHEQSNHSENAIDEEQKKAPNITTSHLQGKQNVRLHNTKKCKKKRPRDDDSDSSIKNFCKKRISGAQKTESEVSEPDDLCYRDYVRLKERKVSEKFKIHRGRVATKDFQKLFRNTMRAFEYKQIPKKPCNEKNLKEAVYDICCNGLSNNAAIIMYFTRSKKVAQIIKIMQKELMIRPNITVSEAFKMNHAPPKYYDKDEIKRFIQLQKQGPQELWDKFENNTTHDLFTRHSDVKTMIIYAATPIDFVGAVKTCNKYAKDNPKEIVLRVCSIIDGDNPISIYNPISKEFKSKFSTLSKC

SEQ ID NO: 20 - protein, 393 aa, human herpesvirus 6A
  ref <308>: DATABASE ACCESSION NUMBER: Uniprot.org/M9TIE8
  ref <309>: DATABASE ENTRY DATE: 2013-06-26
  ref <313>: RELEVANT RESIDUES IN SEQ ID NO: (1)..(393)
MCWSFHLFFKAHKARVGARTSFLTEMERGSRDHHRDHRDHREHRETREPPTLAFHMKSWKTINKSLKAFAKLLKENATVTFTPQPSIIIQSAKNHLVQKLTIQAECLFLSDTDRFLTKTINNHIPLFESFMNIISNPEVTKMYIQHDSDLYTRVLVTASDTCTQASVPCVHGQEVVRDTGRSPLRIDLDHSTVSDVLKWLSPVTKTKRSGKSDALMAHIIVQVNPPTIKFVTEMNELEFSNSNKVIFYDVKNMRFNLSAKNLQQALSMCAVIKTSCSLRTVAAKDCKLILTSKSTLLTVEAFLTQEQLKEESRFERMGKQDDGKGDRSHKNEDGSALASKQEMQYKITNYMVPAKNGTAGSSLFNEKEDSESDDSMHFDYSSNPNPKRQRCVV

SEQ ID NO: 21 - protein, 830 aa, human herpesvirus 6A
  ref <308>: DATABASE ACCESSION NUMBER: Uniprot.org/P28864
  ref <309>: DATABASE ENTRY DATE: 1996-10-01
  ref <313>: RELEVANT RESIDUES IN SEQ ID NO: (1)..(830)
MSKMAVLFLAVFLMNSVLMIYCDPDHYIRAGYNHKYPFRICSIAKGTDLMRFDRDISCSPYKSNAKMSEGFFIIYKTNIETYTFPVRTYKKELTFQSSYRDVGVVYFLDRTVMGLAMPVYEANLVNSHAQCYSAVAMKRPDGTVFSAFHEDNNKNNTLNLFPLNFKSITNKRFITTKEPYFARGPLWLYSTSTSLNCIVTEATAKAKYPFSYFALTTGEIVEGSPFFNGSNGKHFAEPLEKLTILENYTMIEDLMNGMNGATTLVRKIAFLEKADTLFSWEIKEENESVCMLKHWTTVTHGLRAETNETYHFISKELTAAFVAPKESLNLTDPKQTCIKNEFEKIINEVYMSDYNDTYSMNGSYQIFKTTGDLILIWQPLVQKSLMFLEQGSEKIRRRRDVGDVKSRHDILYVQLQYLYDTLKDYINDALGNLAESWCLDQKRTITMLHELSKISPSSIVSEVYGRPISAQLHGDVLAISKCIEVNQSSVQLHKSMRVVDAKGVRSETMCYNRPLVTFSFVNSTPEVVPGQLGLDNEILLGDHRTEECEIPSTKIFLSGNHAHVYTDYTHTNSTPIEDIEVLDAFIRLKIDPLENADFKVLDLYSPDELSRANVFDLENILREYNSYKSALYTIEAKIATNTPSYVNGINSFLQGLGAIGTGLGSVISVTAGALGDIVGGVVSFLKNPFGGGLMLILAIVVVVIIIVVFVRQRHVLSKPIDMMFPYATNPVTTVSSVTGTTVVKTPSVKDVDGGTSVAVSEKEEGMADVSGQVSDDEYSQEDALKMLKAIKSLDESYRRKPSSSESHASKPSLIDRIRYRGYKSVNVEEA

SEQ ID NO: 22 - protein, 723 aa, human herpesvirus 6A
  ref <308>: DATABASE ACCESSION NUMBER: GenBank/YP_009458641
  ref <309>: DATABASE ENTRY DATE: 2019-01-18
  ref <313>: RELEVANT RESIDUES IN SEQ ID NO: (1)..(689)
MMPLTVRAGHAPYRLPISNYWWLLLGRHSLRHVHSYLRLHKGLRIPLPWPEQECLHLHPKPYKCLLRYPSITRQPHLLQGWPTKSSLWFDPKPYHPSADSKLLPLGLITLSACSTRVSQPTHWSGFHVTDLSLSWLTGSSPWLVLLQGQGGSLFCHDVLQGRLYVLSHSVSLFLKTGLRHCEAIYRAPLWRDRPLPSLWTCHDPDKAFLPTLLARSARRGLAAFYALWRLHLGSRSELSHPVLEWERTELVLTDWRRGRPCTHLPSDSRSQRATSSGATDTRDAATEEAAGGKKEAEGGGPSVAEHFARCRPLLDELCGEGGWLPFAFLSTSPHVWLILTEGGPVLAIDVNDTSVWRIADDLELLRRLGSLLLLSGLWLPLRPPSGSGEAVGEPGYEEEERRGRASTASATAATSTRGPTRPTRVTRKGRVATGGVHLPARPESEEQTDGHHGRQESSHDHQRGGSGRGHRDDGAHRHANDKTEPQQRGEHEERKQTDSGRHEHAQESQVARRDEEGTEQGGSGRSCGGATQTYGGRGRHDSCPSIPLSVPGPDPRLWVPPPHLLFPSPLPPMTPVDDEPSVRPRCPPSPAEEPPTCRPRPPRPSSDTPLSAVSRPSAPPVPPPSTARVRFFLSSSSSPSYSPSPLSPPSPVSPSSPRSPFIPPIRSPGLRAKPRVSSGHPAAFPPAPSSAPARSKTVPSGPSSASPSAPCVSKSQPPAAHTA

SEQ ID NO: 23 - protein, 97 aa, human herpesvirus 6B
  ref <308>: DATABASE ACCESSION NUMBER: Uniprot.org/B7XBD2
  ref <309>: DATABASE ENTRY DATE: 2009-03-03
  ref <313>: RELEVANT RESIDUES IN SEQ ID NO: (1)..(97)
MAIGFIGSSPDAELSSENSRISSSVLLGCLLCCTDWSAVVPGKTETFRKPFVAIMIKKLKSCFAAYLSDLEQGSMCDMANASPTSLELGLSKLDKES

SEQ ID NO: 24 - protein, 830 aa, human herpesvirus 6B
  ref <308>: DATABASE ACCESSION NUMBER: Uniprot.org/A0A0N9LSF3
  ref <309>: DATABASE ENTRY DATE: 2016-01-20
  ref <313>: RELEVANT RESIDUES IN SEQ ID NO: (1)..(830)
MSKMRVLFLAVFLMNSVLMIYCDSDDYIRAGYNHKYPFRICSIAKGTDLMRFDRDISCSPYKSNAKMSEGFFIIYKTNIETYTFPVRTYKNELTFPTSYRDVGVVYFLDRTVMGLAMPVYEANLVNSRAQCYSAVAIKRPDGTVFSAYHEDNNKNETLELFPLNFKSVTNKRFITTKEPYFARGPLWLYSTSTSLNCIVTEATAKAKYPFSYFALTTGEIVEGSPFFDGSNGKHFAEPLEKLTILENYTMIEDLMNGMNGATTLVRKIAFLEKGDTLFSWEIKEENESVCMLKHWTTVTHGLRAETDETYHFISKELTAAFVASKESLNLTDPKQTCIKNEFEKIITDVYMSDYNDAYSMNGSYQIFKTTGDLILIWQPLVQKSLMVLEQGSVNLRRRRDLVDVKSRHDILYVQLQYLYDTLKDYINDALGNLAESWCLDQKRTITMLHELSKISPSSIVSEVYGRPISAQLHGDVLAISKCIEVNQSSVQLYKSMRVVDAKGVRSETMCYNRPLVTFSFVNSTPEVVLGQLGLDNEILLGDHRTEECEIPSTKIFLSGNHAHVYTDYTHTNSTPIEDIEVLDAFIRLKIDPLENADFKLLDLYSPDELSRANVFDLENILREYNSYKSALYTIEAKIATNTPSYVNGINSFLQGLGAIGTGLGSVISVTAGALGDIVGGVVSFLKNPFGGGLMLILAIVVVVIIIVVFVRQKHVLSKPIDMMFPYATNPVTTVSSVTGTTVVKTPSVKDADGGTSVAVSEKEEGMADVSGQISGDEYSQEDALKMLKAIKSLDESYRRKPSSSESHASKPSLIDRIRYRGYKSVNVEEA

SEQ ID NO: 25 - protein, 1199 aa, human herpesvirus 7
  ref <308>: DATABASE ACCESSION NUMBER: GenBank/YP_073824
  ref <309>: DATABASE ENTRY DATE: 2018-08-13
  ref <313>: RELEVANT RESIDUES IN SEQ ID NO: (1)..(1199)
MERSGATSPMLIVGQPSETMASNDERHIEYHPDTTVEETIRDILQDSLKCNPSFDNTLFQELDSFDFLDSISSNDIATANASYSTNVEEGASSDIKLAAAQSISACIQGILKSVNAAMDQEKADIEDCLINHAGILTNDRHMLTDLALEQLSQMLNVNLISGASSGFISFYEDILSGEQLEFFLWCESRYTVYACDKFDEIVKKVIADCREYLSDLRNNMDREFIKCIKKIISMGNFISSNEKLNRCATMLLMMGKNRDITDINISNQAFYIKTNKIKKDLLECKFEIIEKNSRIAQTMKDFAIKQMNQMFMSMCDETFLKIHFNCINLIEAAKNLGAAVLQCIVICSKEFSCKKFRANRKEFKITLMHMINDACDQIEKLYDVTGEVTVLNSISIRNGYVTGNGHHSSTLLESHLYNSDSTPPEIEDFNDYGKYSEECSETSEQSYQSVHQNVSPTQCVSKSEQYNVQSNFSDICQTIKTTLAVSQASDESHSKNTTGKNPEVCTREYKLDFFEEIDTQNLAKKVVSKDSPDCLMYPAETNPGMSTEESEVNVSEGTDTQDFSRNTICRFLSDNPIISVEKNSEVNAIKNETGLSEAAATQDFFKEATMKSCRDTKADQILTGQRKTIKNKQKVSKNNEAFNMSLYNLKRNEETYQDKNSLNFSNEFPHTTFSRRDKKTDNQAGILMERSVTEEICVNNSLTFSNEFPPTFSRTDKKTNSPAGILMERSIFNKDTQDKEQYFELFTMTNHTLDNQNKGHNLSEDKTDVKIKTDKSLSGQEEMIKNKQSVSQGNKTSNVNFHNLNRNENKYKDGKDLIIPDKFPNETFYIRDKKKDISTNRAVERSTADVDTESDVEKVAAEGLILLEQNSNNNSRDTDNTNLVISEMETYGYDTDKSTIYESESSSTDTNSTFSSVSLTSFSDSQTTAHDFSAVKKKQLKRKSESKLKTSKAKKKKLIVKEGCNNMELNSCTDTDNENMSNFNTEMSTDSIPYRSKLINNSDNCENNGFPSVVFTIPIRSIRGVTRGVRVKIVPKKNCLWFLRKTHKVNIPDTGMKKDENKELTKQASYCFNNHFIPIRSNDEEYRRENVSYIFPKLQENFIEVDSISETKTMVTNIIMDNFMEITEMIKNGINRHYEELKKRYANKNVSEMNSFILEQSVDTQNYGKENTDSDQTYVDFLEGYPQGYDPFLENAQIY

SEQ ID NO: 26 - protein, 1205 aa, human herpesvirus 7
  ref <308>: DATABASE ACCESSION NUMBER: GenBank/YP_073823.1
  ref <309>: DATABASE ENTRY DATE: 2018-08-13
  ref <313>: RELEVANT RESIDUES IN SEQ ID NO: (1)..(1205)
MSTTADVVREAMTLSGLYNEIHDHTKPENEITYSNFNNQEIVTSKTLQDPRRAIPLKSRSTFTSESSPNTTETGQFRFASELPHQGLQSLYYNYPQASGENFISLPYQPQSSVDIFNNLDYKNRELCHNSFDTTYRQHHYRGRNNRGKYDSRNYCNPNSKYQNHYGKHFHRPYNRRGRGYAKFHDFSNRNEFTFPEYMQRDQSYDSYNSDFRHNPYRDNYQQLCPNYGFQSRDYSMAFSQIPSSSMITDSQNLQKECISSGLTLSKKSEYSQNTPIQTNHKETKLISNVNNLEKPARKKTDKDKTPQNEDVVSLSYIPSSHKMDLIKRIYNADIVPLAKDSLSSTSDVDKCKYNKSSQEKTFMKARTRPSTSKEKQITSTTDSKHCPNLDKGSSNLKKKDKSPDEAIENLRKLSKLEPMLSPLPLTPKPDVQESNQRAPTKLINFKRLLEKNDKDISASDIEFIKQSSRCTVSTALNCNSSTVFSDTKCTGKNSYRVGRPRQAKTTVNSKIDAKDNDFEGKVTAPCNIPSIDNPPVESEKSNTTDSMCNDNLTQRTRSDSSSTEEGQYKSPVNDYSQSEVEHVHSIENQHMEQIISQTTDEIAQNFVSPEQEFLAKSDTENGLFEFFDNELQCTQNSSETKKFPISDRSMSPKSTRSSSSDSDTDTSSSSSSSSSKCSCKSSSSASSKNSVLSRNSCSSTELKPSYTESPNSNGNRQCDFVSKVDMSEFEFRCGKELNLYKHVESRLQLDQNNEHLYNPRFRPHIRKKRPPSTESESDTNSDLQSQSFSSSKKRRHEHRNTESESCSESTKTRHRQKHRSRSSSDTSRSRSASKSRTCKVCSNNKQDSQSETQCKHLISISRSNSEHSISESTYQSCKNKNSETLRSRSRSSESDCKKRSERSCSNHSWSSGSTQKSKRVDIDFTHGAKSIPNEYEQKKSTNKFFKNKRRNARKRFSDDSDSSMQLFYKKRISGTQKSDSELSDNDLSYREYVKLQEQKQSAKFKVQRGRVPTKDFEKLFRKTIRAFEYKQIPKKPFPEAKLKESVYALCCNGSSKSGALIIYFTRSKTVAEDIKAMQKELMVRPNITMSEPFKMNHAPPRYYDKDSIKNFIELQKKGPHELWENISNSAHTLYTRHSDLKTIIIYAATPIDFFMAAKICNNYAKKRPKEIVLRLSSITDGDNPISIYNPVSRDFLSKYTALCKF

SEQ ID NO: 27 - protein, 364 aa, human herpesvirus 7
  ref <308>: DATABASE ACCESSION NUMBER: GenBank/YP_073767.1
  ref <309>: DATABASE ENTRY DATE: 2018-08-13
  ref <313>: RELEVANT RESIDUES IN SEQ ID NO: (1)..(364)
MDRSNREHHSHRDHREHRDSKEPPTMAFHMKTWKTINKPLRAFAKLLRENTTVTFTPQPAIIIQSAKNHLVLKLIIHAECLYITDTDHFSTKTINNFVPLFDSFMGIISNPDVTKLYIQHDSDLYTRFLVTASDICAQASIPCVNGQEIVRESGKSALRIDLDHSTVTEILKWLAPVTKNKRSNKNEMTLAQIVVQVNPPSIKFLTDLNEIEFAHSGKVVFHDAKCMRLVLSSKNLQQAFSTCAVLKSSCSLRAIAGKEYKLFLIAKNVFLTVEAYLSQEQVKDDPKFERQAKTEEKGEKNHKVEEGNNFFCKQETQHKITSYMVPTKNGGTGTNFFNEKEDSESDDSAHFDYTPNSKRQRCGM

SEQ ID NO: 28 - protein, 822 aa, human herpesvirus 7
  ref <308>: DATABASE ACCESSION NUMBER: GenBank/ BAA13124.1
  ref <309>: DATABASE ENTRY DATE: 2016-07-23
  ref <313>: RELEVANT RESIDUES IN SEQ ID NO: (1)..(822)
MKILFLSVFITFSLQLSLQTEADFVMTGHNQHLPFRICSIATGTDLVRFDREVSCASYGSNIKTTEGILIIYKTKIEAHTFSVRTFKKELTFQTTYRDVGTVYFLDRTVTTLPMPIEEVHMVNTEARCLSSISVKRSEEEEYVAYHKDEYVNKTLDLIPLNFKSDTVRRYITTKEPFLRNGPLWFYSTSTSINCIVTDCIAKTKYPFDFFALSTGETVEGSPFYNGINSKTFNEPTEKILFRNNYTMLKTFDDGSKGNFVTLTKMAFLEKGNTIFSWEVQNEESSICLLKHWMTIPHALRAENANSFHFIAQELTASFVTGKSNYTLSDSKYNCINSNYTSILDEIYQTQYNNSHDKNGSYEIFKTEGDLILIWQPLIQRKLTVLENFSNASRKRRKRELETNKDIVYVQLQYLYDTLKDYINTALGKLAEAWCLDQKRTITVLHELSKISPSGIISAVYGKPMSAKLIGDVLAVSKCIEVNQTSVQLHKSMRLTKDSSYDTLRCYSRPLLTYSFANSSKETYLGQLGLDNEILLGNHRTEECEQSNTKIFLSGKFAHIFKDYTYVNSSLITEIEALDAFVDLNIDPLENADFTLLELYTKDELSKANVFDLETILREYNSYKSALHHIETKIATVTPTYIGGIDTFFKGLGALGLGLGAVLGVTAGALGDVVNGVFSFLKNPFGGALTILLTLGVIGLVIFLFLRHKRLAQTPIDILFPYTSKSTNSVLQATQSVQAQVKEPLDSSPPYLKTNKDTEPQGDDITHTNEYSQVEALKMLKAIKLLDESYKKAEIAEAKKSQRPSLLERIQYRGYQKLSTEEL

SEQ ID NO: 29 - protein, 475 aa, Human herpesvirus 7
  ref <308>: DATABASE ACCESSION NUMBER: GenBank/ AAC40802.1
  ref <309>: DATABASE ENTRY DATE: 2009-10-15
  ref <313>: RELEVANT RESIDUES IN SEQ ID NO: (1)..(475)
MTAATTEHFALRAALNRYWWLLLGRHKLSLVCNYVTAHRQQLLPLPWPEQEFLQLDPAPYSNLRNRVAHHLHRGWPAAHNTCFDPRPYFPNAKVKLLPLGSITLTRSFSSDEPHPIGDDVHHSHDRGDYHTVICSWLTGTSPILVLLQGPDGSIYCHDVYRGRLYLVAHSVSLFARLGLRHCEPLYAAPRWKHVPLPSMWVASPPASATLTQTLAVSATHGLDALYSLLKIHRGTPCSLIHPVNGYVLDMILTGRSFQEAPCQNTRTSVKTTPHVMDAVCGGRGSWLSIGYLVKMPHIHLAVTRTCLVTAIDVRQNFLWRVADDALLFLVTGSLLLLSRPTADLTSWSCLQQEPVWRNCLDTRGEQDETEDQEMKQSTSKKQNENKKLNTSKKHTRVSSAIPTFPLSLRETPPEARSPAVLAAATQSHKTRAISTHNATTTIRIPRLPSYLLEARLLSVTAILKDTKKKKTQPQA

SEQ ID NO: 30 - protein, 338 aa, Homo Sapiens
  ref <308>: DATABASE ACCESSION NUMBER: Uniprot.org/P17693
  ref <309>: DATABASE ENTRY DATE: 1990-08-01
  ref <313>: RELEVANT RESIDUES IN SEQ ID NO: (1)..(338)
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 31 - DNA, 167 bp, Epstein-Barr virus
  ref <308>: DATABASE ACCESSION NUMBER: GenBank/ KP195701
  ref <309>: DATABASE ENTRY DATE: 2015-04-04
  ref <313>: RELEVANT RESIDUES IN SEQ ID NO: (1)..(167)
  ref <308>: DATABASE ACCESSION NUMBER: GenBank/KP195701
  ref <309>: DATABASE ENTRY DATE: 2015-04-04
  ref <313>: RELEVANT RESIDUES IN SEQ ID NO: (1)..(167)
aggacctacgctgccctagaggttttgctagggaggagacgtgtgtggctgtagccacccgtcccgggtacaagtcccgggtggtgaggacggtgtctgtggttgtcttcccagactctgctttctgccgtcttcggtcaagtaccagctggtggtccgcatgtttt

SEQ ID NO: 32 - DNA, 173 bp, Epstein-Barr virus
  ref <308>: DATABASE ACCESSION NUMBER: GenBank/ KP195701
  ref <309>: DATABASE ENTRY DATE: 2015-04-04
  ref <313>: RELEVANT RESIDUES IN SEQ ID NO: (328)..(500)
  ref <308>: DATABASE ACCESSION NUMBER: GenBank/KP195701
  ref <309>: DATABASE ENTRY DATE: 2015-04-04
  ref <313>: RELEVANT RESIDUES IN SEQ ID NO: (328)..(500)
aggacagccgttgccctagtggtttcggacacaccgccaacgctcagtgcggtgctaccgacccgaggtcaagtcccgggggaggagaagagaggcttcccgcctagagcatttgcaagtcaggattctctaatccctctgggagaagggtattcggcttgtccgctgttttt

SEQ ID NO: 33 - RNA, 22 nt, Epstein-Barr virus
  ref <308>: DATABASE ACCESSION NUMBER: mirBase/MIMAT0000995
  ref <309>: DATABASE ENTRY DATE: 2004-01-01
  ref <313>: RELEVANT RESIDUES IN SEQ ID NO: (4)..(25)
uaaccugaucagccccggaguu

SEQ ID NO: 34 - RNA, 22 nt, Epstein-Barr virus
  ref <308>: DATABASE ACCESSION NUMBER: miBase/MIMAT0000996
  ref <309>: DATABASE ENTRY DATE: 2004-01-01
  ref <313>: RELEVANT RESIDUES IN SEQ ID NO: (6)..(27)
aaauucuguugcagcagauagc

SEQ ID NO: 35 - RNA, 22 nt, Epstein-Barr virus
  ref <308>: DATABASE ACCESSION NUMBER: mirBase/MIMAT0000998
  ref <309>: DATABASE ENTRY DATE: 2004-01-01
  ref <313>: RELEVANT RESIDUES IN SEQ ID NO: (3)..(24)
uaacgggaaguguguaagcaca

SEQ ID NO: 36 - RNA, 24 nt, Epstein-Barr virus
  ref <308>: DATABASE ACCESSION NUMBER: mirBase/MIMAT0000999
  ref <309>: DATABASE ENTRY DATE: 2004-01-01
  ref <313>: RELEVANT RESIDUES IN SEQ ID NO: (6)..(29)
ucuuaguggaagugacgugcugug

SEQ ID NO: 37 - RNA, 22 nt, Epstein-Barr virus
  ref <308>: DATABASE ACCESSION NUMBER: mirBase/MIMAT0001000
  ref <309>: DATABASE ENTRY DATE: 2004-01-01
  ref <313>: RELEVANT RESIDUES IN SEQ ID NO: (3)..(24)
uauuuucugcauucgcccuugc

SEQ ID NO: 38 - RNA, 21 nt, Epstein-Barr virus
  ref <308>: DATABASE ACCESSION NUMBER: mirBase/MIMAT0003410
  ref <309>: DATABASE ENTRY DATE: 2006-01-01
  ref <313>: RELEVANT RESIDUES IN SEQ ID NO: (12)..(32)
accuaguguuaguguugugcu

SEQ ID NO: 39 - RNA, 22 nt, Epstein-Barr virus
  ref <308>: DATABASE ACCESSION NUMBER: GenBank/MIMAT0003412
  ref <309>: DATABASE ENTRY DATE: 2006-01-01
  ref <313>: RELEVANT RESIDUES IN SEQ ID NO: (9)..(30)
gaccugaugcugcuggugugcu

SEQ ID NO: 40 - RNA, 18 nt, Epstein-Barr virus
  ref <308>: DATABASE ACCESSION NUMBER: mirBase/MIMAT0009205
  ref <309>: DATABASE ENTRY DATE: 2006-01-01
  ref <313>: RELEVANT RESIDUES IN SEQ ID NO: (57)..(74)
gugggccgcuguucaccu

SEQ ID NO: 41 - RNA, 22 nt, Epstein-Barr virus
  ref <308>: DATABASE ACCESSION NUMBER: mirBase/MIMAT0003414
  ref <309>: DATABASE ENTRY DATE: 2006-01-01
  ref <313>: RELEVANT RESIDUES IN SEQ ID NO: (18)..(39)
uaagguugguccaauccauagg

SEQ ID NO: 42 - RNA, 22 nt, Epstein-Barr virus
  ref <308>: DATABASE ACCESSION NUMBER: mirBase/MIMAT0004815
  ref <309>: DATABASE ENTRY DATE: 2006-01-01
  ref <313>: RELEVANT RESIDUES IN SEQ ID NO: (15)..(36)
ccuggaccuugacuaugaaaca

SEQ ID NO: 43 - RNA, 23 nt, Epstein-Barr virus
  ref <308>: DATABASE ACCESSION NUMBER: mirBase/MIMAT0003418
  ref <309>: DATABASE ENTRY DATE: 2006-01-01
  ref <313>: RELEVANT RESIDUES IN SEQ ID NO: (49)..(71)
gucacaaucuauggggucguaga

SEQ ID NO: 44 - RNA, 22 nt, Epstein-Barr virus
  ref <308>: DATABASE ACCESSION NUMBER: mirBase/MIMAT0004816
  ref <309>: DATABASE ENTRY DATE: 2006-01-01
  ref <313>: RELEVANT RESIDUES IN SEQ ID NO: (14)..(35)
uacuggacccugaauuggaaac

SEQ ID NO: 45 - RNA, 22 nt, Epstein-Barr virus
  ref <308>: DATABASE ACCESSION NUMBER: mirBase/MIMAT0004817
  ref <309>: DATABASE ENTRY DATE: 2006-01-01
  ref <313>: RELEVANT RESIDUES IN SEQ ID NO: (18)..(39)
gccaccucuuugguucuguaca

SEQ ID NO: 46 - RNA, 24 nt, Epstein-Barr virus
  ref <308>: DATABASE ACCESSION NUMBER: mirBase/MIMAT0003421
  ref <309>: DATABASE ENTRY DATE: 2006-01-01
  ref <313>: RELEVANT RESIDUES IN SEQ ID NO: (14)..(37)
ucagacaguuuggugcgcuaguug

SEQ ID NO: 47 - RNA, 22 nt, Epstein-Barr virus
  ref <308>: DATABASE ACCESSION NUMBER: mirBase/MIMAT0003423
  ref <309>: DATABASE ENTRY DATE: 2006-01-01
  ref <313>: RELEVANT RESIDUES IN SEQ ID NO: (49)..(70)
uccugugguguuuggugugguu

SEQ ID NO: 48 - RNA, 22 nt, Epstein-Barr virus
  ref <308>: DATABASE ACCESSION NUMBER: mirBase/MIMAT0004818
  ref <309>: DATABASE ENTRY DATE: 2006-01-01
  ref <313>: RELEVANT RESIDUES IN SEQ ID NO: (15)..(36)
aaccggcucguggcucguacag

SEQ ID NO: 49 - protein, 408 aa, Homo Sapiens
  ref <308>: DATABASE ACCESSION NUMBER: GenBank/NP_003133
  ref <309>: DATABASE ENTRY DATE: 2020-11-19
  ref <313>: RELEVANT RESIDUES IN SEQ ID NO: (1)..(408)
MAENGDNEKMAALEAKICHQIEYYFGDFNLPRDKFLKEQIKLDEGWVPLEIMIKFNRLNRLTTDFNVIVEALSKSKAELMEISEDKTKIRRSPSKPLPEVTDEYKNDVKNRSVYIKGFPTDATLDDIKEWLEDKGQVLNIQMRRTLHKAFKGSIFVVFDSIESAKKFVETPGQKYKETDLLILFKDDYFAKKNEERKQNKVEAKLRAKQEQEAKQKLEEDAEMKSLEEKIGCLLKFSGDLDDQTCREDLHILFSNHGEIKWIDFVRGAKEGIILFKEKAKEALGKAKDANNGNLQLRNKEVTWEVLEGEVEKEALKKIIEDQQESLNKWKSKGRRFKGKGKGNKAAQPGSGKGKVQFQGKKTKFASDDEHDEHDENGATGPVKRAREETDKEEPASKQQKTENGAGDQ

SEQ ID NO: 50 - protein, 128 aa, Homo Sapiens [sequence omitted]

SEQ ID NO: 51 - protein, 386 aa, Epstein-Barr virus
  ref <308>: DATABASE ACCESSION NUMBER: GenBank/YP_401722
  ref <309>: DATABASE ENTRY DATE: 2018-08-13
  ref <313>: RELEVANT RESIDUES IN SEQ ID NO: (1)..(386)
MEHDLERGPPGPRRPPRGPPLSSSLGLALLLLLLALLFWLYIVMSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWMYYHGQRHSDEHHHDDSLPHPQQATDDSGHESDSNSNEGRHHLLVSGAGDGPPLCSQNLGAPGGGPDNGPQDPDNTDDNGPQDPDNTDDNGPHDPLPQDPDNTDDNGPQDPDNTDDNGPHDPLPHSPSDSAGNDGGPPQLTEEVENKGGDQGPPLMTDGGGGHSHDSGHGGGDPHLPTLLLGSSGSGGDDDDPHGPVQLSYYD

SEQ ID NO: 52 - protein, 497 aa, Epstein-Barr virus
  ref <308>: DATABASE ACCESSION NUMBER: GenBank/QHE24167
  ref <309>: DATABASE ENTRY DATE: 2020-01-18
  ref <313>: RELEVANT RESIDUES IN SEQ ID NO: (1)..(497)
MGSLEMVPMGAGPPSPGGDPDGYDGGNNSQYPSASGSSGNTPTPPNDEERESNEEPPPPYEDPYWGNGDRHSDYQPLGTQDQSLYLGLQHDGNDGLPPPPYSPRDDSSQHIYEEAGRGSMNPVCLPVIVAPYLFWLAAIAASCFTASVSTVVTATGLALSLLLLAAVASSYAAAQRKLLTPVTVLTAVVTFFAICLTWRIEDPPFNSLLFALLAAAGGLQGIYVLVMLVLLILAYRRRWRRLTVCGGIMFLACVLVLIVDAVLQLSPLLGAVTVVSMTLLLLAFVLWLSSPGGLGTLGAALLTLAAALALLASLILGTLNLTTMFLLMLLWTLVVLLICSSCSSCPLSKILLARLFLYALALLLLASALIAGGSILQTNFKSLSSTEFIPNLFCMLLLIVAGILFILAILTEWGSGNRTYGPVFMCLGGLLTMVAGAVWLTVMSNTLLSAWILTAGFLIFLIGFALFGVIRCCRYCCYYCLTLESEERPPTPYRNTV

SEQ ID NO: 53 - protein, 170 aa, Epstein-Barr virus
  ref <308>: DATABASE ACCESSION NUMBER: GenBank/YP_401634
  ref <309>: DATABASE ENTRY DATE: 2018-08-13
  ref <313>: RELEVANT RESIDUES IN SEQ ID NO: (1)..(170)
MERRLVVTLQCLVLLYLAPECGGTDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 54 - protein, 301 aa, Herpes simplex virus 1
  ref <308>: DATABASE ACCESSION NUMBER: GenBank/ALO18646
  ref <309>: DATABASE ENTRY DATE: 2015-11-21
  ref <313>: RELEVANT RESIDUES IN SEQ ID NO: (1)..(301)
MTSRRSVKSGPREVPRDEYEDLYYTPSSGMASPDSPPDTSRRGALQTRSRQRGEVRFVQYDESDYALYGGSSSEDDEHPEVPRTRRPVSGAVLSGPGPARAPPPPAGSGGAGRTPTTAPRAPRTQRVATKAPAAPAAETTRGRKSAQPESAALPDAPASTAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVE

SEQ ID NO: 55 - protein, 904 aa, Herpes simplex virus 1
  ref <301>: AUTHORS: Pachl et al.
  ref <302>: TITLE: Expression of Cell-Associated and Secreted Forms of Herpes Simplex Virus Type 1 Glycoprotein gB in Mammalian Cells
  ref <303>: JOURNAL: Journal of Virology
  ref <304>: VOLUME: 61
  ref <305>: ISSUE: 2
  ref <306>: PAGES: 315-325
  ref <307>: DATE: 1987-02-01
  ref <313>: RELEVANT RESIDUES IN SEQ ID NO: (1)..(30)
  ref <308>: DATABASE ACCESSION NUMBER: GenBank/AAA45778
  ref <309>: DATABASE ENTRY DATE: 1993-08-02
  ref <313>: RELEVANT RESIDUES IN SEQ ID NO: (1)..(30)
MRQGAPARGCRWFVVWALLGLTLGVLVASAAPSSPGTPGVAAATQAANGGPATPAPPALGAAPTGDPKPKKNKKPKNPTPPRPAGDNATVAAGHATLREHLRDIKAESTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMEHTEHKAKKKGTSALLSAKVTDMVMRKRRNTNYTQVPNKDGDADEDDL

SEQ ID NO: 56 - protein, 511 aa, Herpes Simplex Virus 1
  ref <308>: DATABASE ACCESSION NUMBER: genBank/AAA45779
  ref <309>: DATABASE ENTRY DATE: 1993-08-02
  ref <313>: RELEVANT RESIDUES IN SEQ ID NO: (1)..(511)
MAPGRVGLAVVLWGLLWLGAGVAGGSETASTGPTITAGAVTNASEAPTSGSPGSAASPEVTPTSTPNPNNVTQNKTTPTEPASPPTTPKPTSTPKSPPTSTPDPKPKNNTTPAKSGRPTKPPGPVWCDRRDPLARYGSRVQIRCRFRNSTRMEFRLQIWRYSMGPSPPIAPAPDLEEVLTNITAPPGGLLVYDSAPNLTDPHVLWAEGAGPGADPPLYSVTGPLPTQRLIIGEVTPATQGMYYLAWGRMDSPHEYGTWVRVRMFRPPSLTLQPHAVMEGQPFKATCTAAAYYPRNPVEFDWFEDDRQVFNPGQIDTQTHEHPDGFTTVSTVTSEAVGGQVPPRTFTCQMTWHRDSVTFSRRNATGLALVLPRPTITMEFGVRHVVCTAGCVPEGVTFAWFLGDDPSPAAKSAVTAQESCDHPGLATVRSTLPISYDYSEYICRLTGYPAGIPVLEHHGSHQPPPRDPTERQVIEAIEWVGIGIGVLAAGVLVVTAIVYVVRTSQSRQRHRR

SEQ ID NO: 57 - protein, 234 aa, Herpes simplex virus 1
  ref <308>: DATABASE ACCESSION NUMBER: GenBank/YP_009137132
  ref <309>: DATABASE ENTRY DATE: 2018-08-13
  ref <313>: RELEVANT RESIDUES IN SEQ ID NO: (1)..(234)
MASEAAQPDAGLWSAGNAFADPPPPYDSLSGRNEGPFVVIDLDTPTDPPPPYSAGPLLSVPIPPTSSGEGEASERGRSRQAAQRAARRARRRAERRAQRRSFGPGGLLATPLFLPETRLVAPPDITRDLLSGLPTYAEAMSDHPPTYATVVAVRSTEQPSGALAPDDQRRTQNSGAWRPPRVNSRELYRAQRAARGSSDHAPYRRQGCCGVVWRHAVFGVVAIVVVIILVFLWR

SEQ ID NO: 58 - protein, 904 aa, Herpes simplex virus 2
  ref <308>: DATABASE ACCESSION NUMBER: GenBank/AIL27719
  ref <309>: DATABASE ENTRY DATE: 2015-08-26
  ref <313>: RELEVANT RESIDUES IN SEQ ID NO: (1)..(904)
MRGGGLICALVVGALVAAVASAAPRALGGSGGSGGVAATVAANGGPASRPPPVPSPATTKARKQKTKKPPKRPEATPPPDVNATVAAGHATLRAHLRDIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTEYSLSRVDLGDCIGRDAREAIDRMFARKYNATHIKVGQPQYYLATGGFLIAYQPLLSNTLAELYVREYIREQDRKPRNATAAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 59 - protein, 300 aa, Herpes simplex virus 2
  ref <301>: AUTHORS: Bellner et al.
  ref <302>: TITLE: A Proinflammatory Peptide from Herpes Simplex Virus Type 2 Glycoprotein G A;;ects Neutrophil, Monocyte, and NK Cell Functions
  ref <303>: JOURNAL: The Journal of Immunology
  ref <304>: VOLUME: 174
  ref <306>: PAGES: 2235-2241
  ref <307>: DATE: 2005
  ref <313>: RELEVANT RESIDUES IN SEQ ID NO: (1)..(300)
  ref <308>: DATABASE ACCESSION NUMBER: GenBank/ABU45441
  ref <309>: DATABASE ENTRY DATE: 2007-11-29
  ref <313>: RELEVANT RESIDUES IN SEQ ID NO: (1)..(300)
MHAIAPRLLLLFVLSGLPGTRGGSGVPGPINPPNDDVVFPGGSPVAQYCYAYPRLDDPGPLGSADAGRQDLPRHVVRHEPRGRSFLTGGLVLLAPPIRGFGAPNVTYAARVTYYRLTRACRQPILLRQYGGCRGGSPPSPKTCGSYTYTYQGGGPPTRYALVNASLLVPIWDRAAETFEYQIELGGELHVGLLWVKAGGEGPGPTAPPQAARAEGGPCVPPVPAGRPWRAVPPVWYSAPNPGFRGLRFRERCLPPQTPAAPSDLPRVAFAPQSLLVGITGRTFIRMARPTGDVGVLPPHW

SEQ ID NO: 60 - protein, 16 aa, Herpes simplex virus 2
  ref <301>: AUTHORS: Bellner et al.
  ref <302>: TITLE: A Proinflammatory Peptide from Herpes Simplex Virus Type 2 Glycoprotein G Affects Neutrophil, Monocyte, and NK Cell Functions
  ref <303>: JOURNAL: The Journal of Immunology
  ref <304>: VOLUME: 174
  ref <306>: PAGES: 2235-2241
  ref <307>: DATE: 2005
  ref <313>: RELEVANT RESIDUES IN SEQ ID NO: (190)..(205)
  ref <308>: DATABASE ACCESSION NUMBER: GenBank/ABU45441
  ref <309>: DATABASE ENTRY DATE: 2007-11-29
  ref <313>: RELEVANT RESIDUES IN SEQ ID NO: (190)..(205)
VGLLWVKAGGEGPGPT

SEQ ID NO: 61 - protein, 868 aa, Varicella Zoster virus
  ref <308>: DATABASE ACCESSION NUMBER: GenBank/AAK01041.1
  ref <309>: DATABASE ENTRY DATE: 2016-06-10
  ref <313>: RELEVANT RESIDUES IN SEQ ID NO: (1)..(868)
MFVTAVVSVSPSSFYESLQVEPTQSEDITRSAHLGDGDEIREAIHKSQDAETKPTFYVCPPPTGSTIVRLEPTRTCPDYHLGKNFTEGIAVVYKENIAAYKFKATVYYKDVIVSTAWAGSSYTQITNRYADRVPIPVSEITDTIDKFGKCSSKATYVRNNHKVEAFNEDKNPQDMPLIASKYNSVGSKAWHTTNDTYMVAGTPGTYRTGTSVNCIIEEVEARSIFPYDSFGLSTGDIIYMSPFFGLRDGAYREHSNYAMDRFHQFEGYRQRDLDTRALLEPAARNFLVTPHLTVGWNWKPKRTEVCSLVKWREVEDVVRDEYAHNFRFTMKTLSTTFISETNEFNLNQIHLSQCVKEEARAIINRIYTTRYNSSHVRTGDIQTYLARGGFVVVFQPLLSNSLARLYLQELVRENTNHSPQKHPTRNTRSRRSVPVELRANRTITTTSSVEFAMLQFTYDHIQEHVNEMLARISSSWCQLQNRERALWSGLFPINPSALASTILDQRVKARILGDVISVSNCPELGSDTRIILQNSMRVSGSTTRCYSRPLISIVSLNGSGTVEGQLGTDNELIMSRDLLEPCVANHKRYFLFGHHYVYYEDYRYVREIAVHDVGMISTYVDLNLTLLKDREFMPLQVYTRDELRDTGLLDYSEIQRRNQMHSLRFYDIDKVVQYDSGTAIMQGMAQFFQGLGTAGQAVGHVVLGATGALLSTVHGFTTFLSNPFGALAVGLLVLAGLVAAFFAYRYVLKLKTSPMKALYPLTTKGLKQLPEGMDPFAEKPNATDTPIEEIGDSQNTEPSVNSGFDPDKFREAQEMIKYMTLVSAAERQESKARKKNKTSALLTSRLTGLALRNRRGYSRVRTENVTGV

SEQ ID NO: 62 - protein, 623 aa, Varicella Zoster virus
  ref <308>: DATABASE ACCESSION NUMBER: GenBank/AAK01047
  ref <309>: DATABASE ENTRY DATE: 2016-06-10
  ref <313>: RELEVANT RESIDUES IN SEQ ID NO: (1)..(623)
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLAAVVLLCLVIFLICTAKRMRVKAYRVDKSPYNQSMYYAGLPVDDFEDSESTDTEEEFGNAIGGSHGGSSYTVYIDKTR

SEQ ID NO: 63 - protein, 354 aa, Varicella Zoster virus
  ref <308>: DATABASE ACCESSION NUMBER: GenBank/AAK01046
  ref <309>: DATABASE ENTRY DATE: 2016-06-10
  ref <313>: RELEVANT RESIDUES IN SEQ ID NO: (1)..(354)
MFLIQCLISAVIFYIQVTNALIFKGDHVSLQVNSSLTSILIPMQNDNYTEIKGQLVFIGEQLPTGTNYSGTLELLYADTVAFCFRSVQVIRYDGCPRIRTSAFISCRYKHSWHYGNSTDRISTEPDAGVMLKITKPGINDAGVYVLLVRLDHSRSTDGFILGVNVYTAGSHHNIHGVIYTSPSLQNGYSTRALFQQARLCDLPATPKGSGTSLFQHMLDLRAGKSLEDNPWLHEDVVTTETKSVVKEGIENHVYPTDMSTLPEKSLNDPPENLLIIIPIVASVMILTAMVIVIVISVKRRRIKKHPIYRPNTKTRRGIQNATPESDVMLEAAIAQLATIREESPPHSVVNPFVK

SEQ ID NO: 64 - protein, 560 aa, Varicella Zoster Virus
  ref <308>: DATABASE ACCESSION NUMBER: GenBank/AGC94505
  ref <309>: DATABASE ENTRY DATE: 2015-07-14
  ref <313>: RELEVANT RESIDUES IN SEQ ID NO: (1)..(560)
MKRIQINLILTIACIQLSTESQPTPVSITELYTSAATRKPDPAVAPTSAASRKPDPAVAPTSAATRKPDPAVAPTSAATRKPDPAVAPTSAATRKPDPAVAPTSAASRKPDPAVAPTSAASRKPDPAVAPTSAATRKPDPAANTQHSQPPFLYENIQCVHGGIQSIPYFHTFIMPCYMRLTTGQQAAFKQQQKTYEQYSLDPEGSNITRWKSLIRPDLHIEVWFTRHLIDPHRQLGNALIRMPDLPVMLYSNSADLNLINNPEIFTHAKENYVIPDVKTTSDFSVTILSMDATTEGTYIWRVVNTKTKNVISEHSITVTTYYRPNITVVGDPVLTGQTYAAYCNVSKYYPPHSVRVRWTSRFGNIGKNFITDAIQEYANGLFSYVSAVRIPQQKQMDYPPPAIQCNVLWIRDGVSNMKYSAVVTPDVYPFPNVSIGIIDGHIVCTAKCVPRGVVHFVWWVNDSPINHENSEITGVCDQNKRFVNMQSSCPTSELDGPITYSCHLDGYPKKFPPFSAVYTYDASTYATTFSVVAVIIGVISILGTLGLIAVIATLCIRCCS

SEQ ID NO: 65 - protein, 841 aa, Varicella Zoster virus
  ref <308>: DATABASE ACCESSION NUMBER: GenBank/AAK01042
  ref <309>: DATABASE ENTRY DATE: 2016-06-10
  ref <313>: RELEVANT RESIDUES IN SEQ ID NO: (1)..(841)
MFALVLAVVILPLWTTANKSYVTPTPATRSIGHMSALLREYSDRNMSLKLEAFYPTGFDEELIKSLHWGNDRKHVFLVIVKVNPTTHEGDVGLVIFPKYLLSPYHFKAEHRAPFPAGRFGFLSHPVTPDVSFFDSSFAPYLTTQHLVAFTTFPPNPLVWHLERAETAATAERPFGVSLLPARPTVPKNTILEHKAHFATWDALARHTFFSAEAIITNSTLRIHVPLFGSVWPIRYWATGSVLLTSDSGRVEVNIGVGFMSSLISLSSGPPIELIVVPHTVKLNAVTSDTTWFQLNPPGPDPGPSYRVYLLGRGLDMNFSKHATVDICAYPEESLDYRYHLSMAHTEALRMTTKADQHDINEESYYHIAARIATSIFALSEMGRTTEYFLLDEIVDVQYQLKFLNYILMRIGAGAHPNTISGTSDLIFADPSQLHDELSLLFGQVKPANVDYFISYDEARDQLKTAYALSRGQDHVNALSLARRVIMSIYKGLLVKQNLNATERQALFFASMILLNFREGLENSSRVLDGRTTLLLMTSMCTAAHATQAALNIQEGLAYLNPSKHMFTIPNVYSPCMGSLRTDLTEEIHVMNLLSAIPTRPGLNEVLHTQLDESEIFDAAFKTMMIFTTWTAKDLHILHTHVPEVFTCQDAAARNGEYVLILPAVQGHSYVITRNKPQRGLVYSLADVDVYNPISVVYLSKDTCVSEHGVIETVALPHPDNLKECLYCGSVFLRYLTTGAIMDIIIIDSKDTERQLAAMGNSTIPPFNPDMHGDDSKAVLLFPNGTVVTLLGFERRQAIRMSGQYLGASLGGAFLAVVGFGIIGWMLCGNSRLREYNKIPLT

SEQ ID NO: 66 - protein, 160 aa, Varicella Zoster virus
  ref <308>: DATABASE ACCESSION NUMBER: GenBank/ AGC94560
  ref <309>: DATABASE ENTRY DATE: 2015-07-14
  ref <313>: RELEVANT RESIDUES IN SEQ ID NO: (1)..(160)
MASHKWLLQMIVFLKTITIAYCLHLQDDTPLFFGAKPLSDVSLIITEPCVSSVYEAWDYAAPPVSNLSEALSGIVVKTKCPVPEVILWFKDKQMAYWTNPYVTLKGLAQSVGEEHKSGDIRDALLDALSGVWVDSTPSSTNIPENGCVWGADRLFQRVCQ

SEQ ID NO: 67 - protein, 204 aa, Kaposi Sarcoma virus
  ref <308>: DATABASE ACCESSION NUMBER: GenBank/AAB61701
  ref <309>: DATABASE ENTRY DATE: 1997-06-27
  ref <313>: RELEVANT RESIDUES IN SEQ ID NO: (1)..(204)
MCWFKLWSLLLVGSLLVSGTRGKLPDAPEFEKDLLIQRLNWMLWVIDECFRDLCYRTGICKGILEPAAIFHLKLPAINDTDHCGLIGFNETSCLKKLADGFFEFEVLFKFLTTEFGKSVINVDVMELLTKTLGWDIQEELNKLTKTHYSPPKFDRGLLGRLQGLKYWVRHFASFYVLSAMEKFAGQAVRVLDSIPDVTPDVHDK

SEQ ID NO: 68 - protein, 196 aa, Kaposi Sarcoma virus
  ref <301>: AUTHORS: Chandran et al
  ref <302>: TITLE: Reactivities of human sera with human herpesvirus-8- infected BCBL-1 cells and identification of HHV-8-specific proteins and glycoproteins and the encoding cDNAs
  ref <303>: JOURNAL: Virology
  ref <304>: VOLUME: 243
  ref <305>: ISSUE: 1
  ref <306>: PAGES: 208-217
  ref <307>: DATE: 1998-03-30
  ref <313>: RELEVANT RESIDUES IN SEQ ID NO: (1)..(196)
  ref <308>: DATABASE ACCESSION NUMBER: GenBank/F5HB98
  ref <309>: DATABASE ENTRY DATE: 2011-06-28
  ref <313>: RELEVANT RESIDUES IN SEQ ID NO: (1)..(196)
MSSTQIRTEIPVALLILCLCLVACHANCPTYRSHLGFWQEGWSGQVYQDWLGRMNCSYENMTALEAVSLNGTRLAAGSPSSEYPNVSVSVEDTSASGSGEDAIDESGSGEEERPVTSHVTFMTQSVQATTELTDALISAFSGSYSSGEPSRTTRIRVSPVAENGRNSGASNRVPFSATTTTTRGRDAHYNAEIRTH

SEQ ID NO: 69 - protein, 550 aa, Kaposi Sarcoma virus
  ref <308>: DATABASE ACCESSION NUMBER: GenBank/QLI56341
  ref <309>: DATABASE ENTRY DATE: 2020-07-22
  ref <313>: RELEVANT RESIDUES IN SEQ ID NO: (1)..(550)
MAFLRQTLWILWTFTMVIGQDNEKCSQKTLIGYRLKMSRDGDIAVGETVELRCRSGYTTYARNITATCLQGGTWSEPTATCNKKSCPNPGEIQNGKVIFHGGQDALKYGANISYVCNEGYFLVGREYVRYCMIGASGQMAWSSSPPFCEKEKCHRPKIENGDFKPDKDYYEYNDAVHFECNEGYTLVGPHSIACAVNNTWTSNMPTCELAGCKFPSVTHGYPIQGFSLTYKHKQSVTFACNDGFVLRGSPTITCNVTEWDPPLPKCVLEDIDDPNNSNPGRLHPTPNEKPNGNVFQRSNYTEPPTKPEDTHTAATCDTNCEQPPKILPTSEGFNETTTSNTITKQLEDEKTTSQPNTHITSALTSMKAKGNFTNKTNNSTDLHIASTPTSQDDATPSIPSVQTPNYNTNAPTRTLTSLHIEEGPSNSTTSEKATASTLSHNSHKNDTGGIYTTLNKTTQLPSTNKPTNSQAKSSTKPRVETHNKTTSNPAISLTDSADVPQRPREPTLPPIFRPPASKNRYLEKQLVIGLLTAVALTCGLITLFHYLFFR

SEQ ID NO: 70 - RNA, 22 nt, Kaposi Sarcoma virus
  ref <308>: DATABASE ACCESSION NUMBER: mirBase.org/MIMAT0015214
  ref <309>: DATABASE ENTRY DATE: 2005-04-12
  ref <313>: RELEVANT RESIDUES IN SEQ ID NO: (41)..(62)
gcagcaccuguuuccugcaacc

SEQ ID NO: 71 - RNA, 21 nt, Kaposi Sarcoma virus
  ref <308>: DATABASE ACCESSION NUMBER: mirBase.org/MIMAT0015215
  ref <309>: DATABASE ENTRY DATE: 2005-04-12
  ref <313>: RELEVANT RESIDUES IN SEQ ID NO: (58)..(78)
gaucuuccagggcuagagcug

SEQ ID NO: 72 - RNA, 21 nt, Kaposi Sarcoma virus
  ref <308>: DATABASE ACCESSION NUMBER: mirBase.org/MIMAT0002194
  ref <309>: DATABASE ENTRY DATE: 2005-04-12
  ref <313>: RELEVANT RESIDUES IN SEQ ID NO: (48)..(68)
ucgcggucacagaaugugaca

SEQ ID NO: 73 - RNA, 22 nt, Kaposi Sarcoma virus
  ref <308>: DATABASE ACCESSION NUMBER: mirBase.org/MIMAT0002192
  ref <309>: DATABASE ENTRY DATE: 2005-04-12
  ref <313>: RELEVANT RESIDUES IN SEQ ID NO: (45)..(66)
uagaauacugaggccuagcuga

SEQ ID NO: 74 - RNA, 23 nt, Kaposi Sarcoma virus
  ref <308>: DATABASE ACCESSION NUMBER: mirBase.org/MIMAT0002190
  ref <309>: DATABASE ENTRY DATE: 2005-04-12
  ref <313>: RELEVANT RESIDUES IN SEQ ID NO: (46)..(68)
uaggaugccuggaacuugccggu

SEQ ID NO: 75 - RNA, 22 nt, Kaposi Sarcoma virus
  ref <308>: DATABASE ACCESSION NUMBER: mirBase.org/MIMAT0002189
  ref <309>: DATABASE ENTRY DATE: 2005-04-12
  ref <313>: RELEVANT RESIDUES IN SEQ ID NO: (37)..(58)
ugaugguuuucgggcuguugag

SEQ ID NO: 76 - RNA, 20 nt, Kaposi Sarcoma virus
  ref <308>: DATABASE ACCESSION NUMBER: mirBase.org/MIMAT0002187
  ref <309>: DATABASE ENTRY DATE: 2005-04-12
  ref <313>: RELEVANT RESIDUES IN SEQ ID NO: (47)..(66)
ugaucccauguugcuggcgc

SEQ ID NO: 77 - RNA, 21 nt, Kaposi Sarcoma virus
  ref <308>: DATABASE ACCESSION NUMBER: mirBase.org/MIMAT0002186
  ref <309>: DATABASE ENTRY DATE: 2005-04-12
  ref <313>: RELEVANT RESIDUES IN SEQ ID NO: (46)..(66)
cuaggcgcgacugagagagca

SEQ ID NO: 78 - RNA, 22 nt, Kaposi Sarcoma virus
  ref <308>: DATABASE ACCESSION NUMBER: mirBase.org/MIMAT0002185
  ref <309>: DATABASE ENTRY DATE: 2005-04-12
  ref <313>: RELEVANT RESIDUES IN SEQ ID NO: (41)..(62)
cuggguauacgcagcugcguaa

SEQ ID NO: 79 - RNA, 22 nt, Kaposi Sarcoma virus
  ref <308>: DATABASE ACCESSION NUMBER: mirBase.org/MIMAT0002179
  ref <309>: DATABASE ENTRY DATE: 2005-04-12
  ref <313>: RELEVANT RESIDUES IN SEQ ID NO: (46)..(67)
uaguguuguccccccgaguggc

SEQ ID NO: 80 - RNA, 22 nt, Kaposi Sarcoma virus
  ref <308>: DATABASE ACCESSION NUMBER: mirBase.org/MIMAT0002181
  ref <309>: DATABASE ENTRY DATE: 2005-07-05
  ref <313>: RELEVANT RESIDUES IN SEQ ID NO: (46)..(67)
uuaaugcuuagccuguguccga

SEQ ID NO: 81 - RNA, 22 nt, Kaposi Sarcoma virus
  ref <308>: DATABASE ACCESSION NUMBER: mjRBase/MIMAT0002181
  ref <309>: DATABASE ENTRY DATE: 2006-03-15
  ref <313>: RELEVANT RESIDUES IN SEQ ID NO: (59)..(80)
ugggggagggugcccugguuga

SEQ ID NO: 82 - protein, 437 aa, Homo Sapiens
  ref <308>: DATABASE ACCESSION NUMBER: GenBank/AAB59531
  ref <309>: DATABASE ENTRY DATE: 1999-06-16
  ref <313>: RELEVANT RESIDUES IN SEQ ID NO: (1)..(437)
MSWSLHPRNLILYFYALLFLSSTCVAYVATRDNCCILDERFGSYCPTTCGIADFLSTYQTKVDKDLQSLEDILHQVENKTSEVKQLIKAIQLTYNPDESSKPNMIDAATLKSRIMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQAGDV

SEQ ID NO: 83 - protein, 622 aa, Homo Sapiens
  ref <308>: DATABASE ACCESSION NUMBER: GenBank/AAC63054
  ref <309>: DATABASE ENTRY DATE: 1998-10-07
  ref <313>: RELEVANT RESIDUES IN SEQ ID NO: (1)..(622)
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 84 - protein, 295 aa, Homo Sapiens
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREIFYIIGAVVFVVIILVIILAISLHKCRKAGVGQSWKENSPLNVS

SEQ ID NO: 85 - protein, 402 aa, Homo Sapiens [sequence omitted]

SEQ ID NO: 86 - protein, 317 aa, Homo Sapiens
  ref <308>: DATABASE ACCESSION NUMBER: GenBank/3H32_F
  ref <309>: DATABASE ENTRY DATE: 2020-12-01
  ref <313>: RELEVANT RESIDUES IN SEQ ID NO: (1)..(317)
KYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQAGDV

SEQ ID NO: 87 - protein, 2813 aa, Homo Sapiens
  ref <308>: DATABASE ACCESSION NUMBER: GenBank/NP_000543
  ref <309>: DATABASE ENTRY DATE: 2021-01-03
  ref <313>: RELEVANT RESIDUES IN SEQ ID NO: (1)..(2813)
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRIALLLMASQEPQRMSRNFVRYVQGLKKKKVIVIPVGIGPHANLKQIRLIEKQAPENKAFVLSSVDELEQQRDEIVSYLCDLAPEAPPPTLPPHMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 88 - protein, 644 aa, Homo Sapiens
  ref <308>: DATABASE ACCESSION NUMBER: GenBank/NP_001095886
  ref <309>: DATABASE ENTRY DATE: 2020-12-14
  ref <313>: RELEVANT RESIDUES IN SEQ ID NO: (1)..(644)
MKLITILFLCSRLLLSLTQESQSEEIDCNDKDLFKAVDAALKKYNSQNQSNNQFVLYRITEATKTVGSDTFYSFKYEIKEGDCPVQSGKTWQDCEYKDAAKAATGECTATVGKRSSTKFSVATQTCQITPAEGPVVTAQYDCLGCVHPISTQSPDLEPILRHGIQYFNNNTQHSSLFMLNEVKRAQRQVVAGLNFRITYSIVQTNCSKENFLFLTPDCKSLWNGDTGECTDNAYIDIQLRIASFSQNCDIYPGKDFVQPPTKICVGCPRDIPTNSPELEETLTHTITKLNAENNATFYFKIDNVKKARVQVVAGKKYFIDFVARETTCSKESNEELTESCETKKLGQSLDCNAEVYVVPWEKKIYPTVNCQPLGMISLMKRPPGFSPFRSSRIGEIKEETTVSPPHTSMAPAQDEERDSGKEQGHTRRHDWGHEKQRKHNLGHGHKHERDQGHGHQRGHGLGHGHEQQHGLGHGHKFKLDDDLEHQGGHVLDHGHKHKHGHGHGKHKNKGKKNGKHNGWKTEHLASSSEDSTTPSAQTQEKTEGPTPIPSLAKPGVTVTFSDFQDSDLIATMMPPISPAPIQSDDDWIPDIQIDPNGLSFNPISDFPDTTSPKCPGRPWKSVSEINPTTQMKESYYFDLTDGLS

SEQ ID NO: 89 - protein, 464 aa, Homo Sapiens
  ref <308>: DATABASE ACCESSION NUMBER: GenBank/NP_000479
  ref <309>: DATABASE ENTRY DATE: 2020-12-16
  ref <313>: RELEVANT RESIDUES IN SEQ ID NO: (1)..(464)
MYSNVIGTVTSGKRKVYLLSLLLIGFWDCVTCHGSPVDICTAKPRDIPMNPMCIYRSPEKKATEDEGSEQKIPEATNRRVWELSKANSRFATTFYQHLADSKNDNDNIFLSPLSISTAFAMTKLGACNDTLQQLMEVFKFDTISEKTSDQIHFFFAKLNCRLYRKANKSSKLVSANRLFGDKSLTFNETYQDISELVYGAKLQPLDFKENAEQSRAAINKWVSNKTEGRITDVIPSEAINELTVLVLVNTIYFKGLWKSKFSPENTRKELFYKADGESCSASMMYQEGKFRYRRVAEGTQVLELPFKGDDITMVLILPKPEKSLAKVEKELTPEVLQEWLDELEEMMLVVHMPRFRIEDGFSLKEQLQDMGLVDLFSPEKSKLPGIVAEGRDDLYVSDAFHKAFLEVNEEGSEAAASTAVVIAGRSLNPNRVTFKANRPFLVFIREVPLNTIIFMGRVANPCVK

SEQ ID NO: 90 - protein, 615 aa, Homo Sapiens
  ref <308>: DATABASE ACCESSION NUMBER: Genbank/AAB59491
  ref <309>: DATABASE ENTRY DATE: 2016-07-26
  ref <313>: RELEVANT RESIDUES IN SEQ ID NO: (1)..(237)
MRALLLLGFLLVSLESTLSIPPWEAPKEHKYKAEEHTVVLTVTGEPCHFPFQYHRQLYHKCTHKGRPGPQPWCATTPNFDQDQRWGYCLEPKKVKDHCSKHSPCQKGGTCVNMPSGPHCLCPQHLTGNHCQKEKCFEPQLLRFFHKNEIWYRTEQAAVARCQCKGPDAHCQRLASQACRTNPCLHGGRCLEVEGHRLCHCPVGYTGPFCDVDTKASCYDGRGLSYRGLARTTLSGAPCQPWASEATYRNVTAEQARNWGLGGHAFCRNPDNDIRPWCFVLNRDRLSWEYCDLAQCQTPTQAAPPTPVSPRLHVPLMPAQPAPPKPQPTTRTPPQSQTPGALPAKREQPPSLTRNGPLSCGQRLRKSLSSMTRVVGGLVALRGAHPYIAALYWGHSFCAGSLIAPCWVLTAAHCLQDRPAPEDLTVVLGQERRNHSCEPCQTLAVRSYRLHEAFSPVSYQHDLALLRLQEDADGSCALLSPYVQPVCLPSGAARPSETTLCQVAGWGHQFEGAEEYASFLQEAQVPFLSLERCSAPDVHGSSILPGMLCAGFLEGGTDACQGDSGGPLVCEDQAAERRLTLQGIISWGSGCGDRNKPGVYTDVAYYLAWIREHTVS

SEQ ID NO: 91 - protein, 216 aa, Homo Sapiens
  ref <308>: DATABASE ACCESSION NUMBER: Genbank/NP_063916
  ref <309>: DATABASE ENTRY DATE: 2020-12-17
  ref <313>: RELEVANT RESIDUES IN SEQ ID NO: (1)..(216)
MRIQDPGKVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 92 - protein, 260 aa, Homo Sapiens
  ref <308>: DATABASE ACCESSION NUMBER: Genbank/NP_009127
  ref <309>: DATABASE ENTRY DATE: 2020-11-19
  ref <313>: RELEVANT RESIDUES IN SEQ ID NO: (1)..(260)
MGRPRPRAAKTWMFLLLLGGAWAGHSRAQEDKVLGGHECQPHSQPWQAALFQGQQLLCGGVLVGGNWVLTAAHCKKPKYTVRLGDHSLQNKDGPEQEIPVVQSIPHPCYNSSDVEDHNHDLMLLQLRDQASLGSKVKPISLADHCTQPGQKCTVSGWGTVTSPRENFPDTLNCAEVKIFPQKKCEDAYPGQITDGMVCAGSSKGADTCQGDSGGPLVCDGALQGITSWGSDPCGRSDKPGVYTNICRYLDWIKKIIGSKG

SEQ ID NO: 93 - protein, 466 aa, Homo Sapiens
  ref <308>: DATABASE ACCESSION NUMBER: Genbank/AAA88040
  ref <309>: DATABASE ENTRY DATE: 1996-02-13
  ref <313>: RELEVANT RESIDUES IN SEQ ID NO: (1)..(466)
MVSQALRLLCLLLGLQGCLAAGGVAKASGGETRDMPWKPGPHRVFVTQEEAHGVLHRRRRANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP

SEQ ID NO: 94 - protein, 488 aa, Homo Sapiens
  ref <308>: DATABASE ACCESSION NUMBER: Genbank/AAA52421
  ref <309>: DATABASE ENTRY DATE: 1994-11-08
  ref <313>: RELEVANT RESIDUES IN SEQ ID NO: (1)..(488)
MGRPLHLVLLSASLAGLLLLGESLFIRREQANNILARVTRANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 95 - protein, 196 aa, Homo Sapiens
  ref <308>: DATABASE ACCESSION NUMBER: 1VSC_B
  ref <309>: DATABASE ENTRY DATE: 2020-12-01
  ref <313>: RELEVANT RESIDUES IN SEQ ID NO: (1)..(196)
  ref <308>: DATABASE ACCESSION NUMBER: Genbank/1VSC_B
  ref <309>: DATABASE ENTRY DATE: 2020-12-01
  ref <313>: RELEVANT RESIDUES IN SEQ ID NO: (1)..(196)
FKIETTPESRYLAQIGDSVSLTCSTTGCESPFFSWRTQIDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVD

SEQ ID NO: 96 - protein, 532 aa, Homo Sapiens
  ref <308>: DATABASE ACCESSION NUMBER: CAA41977
  ref <309>: DATABASE ENTRY DATE: 2006-11-14
  ref <313>: RELEVANT RESIDUES IN SEQ ID NO: (1)..(532)
  ref <308>: DATABASE ACCESSION NUMBER: Genbank/CAA41977
  ref <309>: DATABASE ENTRY DATE: 2006-11-14
  ref <313>: RELEVANT RESIDUES IN SEQ ID NO: (1)..(532)
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTREVTVNVLSPRYEIVIITVVAAAVIMGTAGLSTYLYNRQRKIKKYRLQQAQKGTPMKPNTQATPP

SEQ ID NO: 97 - protein, 610 aa, Homo Sapiens
  ref <308>: DATABASE ACCESSION NUMBER: NP_000441
  ref <309>: DATABASE ENTRY DATE: 2020-01-03
  ref <313>: RELEVANT RESIDUES IN SEQ ID NO: (1)..(610)
  ref <308>: DATABASE ACCESSION NUMBER: Genbank/NP_000441
  ref <309>: DATABASE ENTRY DATE: 2020-01-03
  ref <313>: RELEVANT RESIDUES IN SEQ ID NO: (1)..(610)
MIASQFLSALTLVLLIKESGAWSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVTCRAVRQPQNGSVRCSHSPAGEFTFKSSCNFTCEEGFMLQGPAQVECTTQGQWTQQIPVCEAFQCTALSNPERGYMNCLPSASGSFRYGSSCEFSCEQGFVLKGSKRLQCGPTGEWDNEKPTCEAVRCDAVHQPPKGLVRCAHSPIGEFTYKSSCAFSCEEGFELHGSTQLECTSQGQWTEEVPSCQVVKCSSLAVPGKINMSCSGEPVFGTVCKFACPEGWTLNGSAARTCGATGHWSGLLPTCEAPTESNIPLVAGLSAAGLSLLTLAPFLLWLRKCLRKAKKFVPASSCQSLESDGSYQKPSYIL

SEQ ID NO: 98 - protein, 42 aa, Homo Sapiens [sequence omitted]

SEQ ID NO: 99 - protein, 352 aa, Homo Sapiens
  ref <308>: DATABASE ACCESSION NUMBER: CAA38585
  ref <309>: DATABASE ENTRY DATE: 2008-10-23
  ref <313>: RELEVANT RESIDUES IN SEQ ID NO: (1)..(352)
  ref <308>: DATABASE ACCESSION NUMBER: Genbank/CAA38585
  ref <309>: DATABASE ENTRY DATE: 2008-10-23
  ref <313>: RELEVANT RESIDUES IN SEQ ID NO: (1)..(352)
MRSLGALLLLLSACLAVSAGPVPTPPDNIQVQENFNISRIYGKWYNLAIGSTCPWLKKIMDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQEPEPILIPRVRRAVLPQEEEGSGGGQLVTEVTKKEDSCQLGYSAGPCMGMTSRYFYNGTSMACETFQYGGCMGNGNNFVTEKECLQTCRTVAACNLPIVRGPCRAFIQLWAFDAVKGKCVLFPYGGCQGNGNKFYSEKECREYCGVPGDGDEELLRFSN

SEQ ID NO: 100 - protein, 582 aa, Homo Sapiens [sequence omitted]

SEQ ID NO: 101 - protein, 1663 aa, Homo Sapiens
  ref <308>: DATABASE ACCESSION NUMBER: NP_000055
  ref <309>: DATABASE ENTRY DATE: 2020-12-12
  ref <313>: RELEVANT RESIDUES IN SEQ ID NO: (1)..(1663)
  ref <308>: DATABASE ACCESSION NUMBER: Genbank/NP_000055
  ref <309>: DATABASE ENTRY DATE: 2020-12-12
  ref <313>: RELEVANT RESIDUES IN SEQ ID NO: (1)..(1663)
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

(19) United States
(12) Patent Application Publication
(10) Pub. No.: US 2022/0170933 A1
(43) Pub. Date: Jun. 2, 2022

(54) METHOD FOR DIAGNOSING AND STAGING HERPESVIRUS-MEDIATED NEURODEGENERATION

(71) Applicant: Richelle Gayle Cutler, Oro Valley, AZ (US)

(72) Inventor: Richelle Gayle Cutler, Oro Valley, AZ (US)

(21) Appl. No.: 17/145,403

(22) Filed: Jan. 11, 2021

Related U.S. Application Data
(60) Provisional application No. 63/120,072, filed on Dec. 1, 2020.

(51) Int. Cl. G01N 33/569; C12Q 1/70

(52) U.S. Cl. CPC G01N 33/56994; C12Q 1/705; G01N 2800/28; G01N 2800/26; G01N 2333/045; C12Q 2600/158; G01N 2333/035; G01N 2333/05; G01N 2333/04; G01N 2333/03; C12Q 2600/112; G01N 2800/56

(57) ABSTRACT

The invention describes a method to identify and stage human herpesvirus-mediated neurodegenerations by measuring secreted herpesvirus protein and RNA levels in a blood sample and comparing that level to an individual's baseline or reference level. Combining herpesvirus secretome levels with other neurodegeneration biomarkers, such as coagulation factors and cell adhesion molecules, can aid in diagnosis and staging of neuropathology. Sensitive and economical ELISA diagnostic kits can detect and quantify the herpesvirus secretome and neurodegeneration biomarkers to diagnose herpesvirus-mediated neurodegeneration, stage disease, and predict disease progression. Finally, this method will elucidate herpesvirus-mediated neurodegenerations and aid in developing therapeutics.

This application claims benefit under 35 U.S.C. section 119(e) of U.S. Provisional Aβ plication No. 63/120,072, filed on Dec. 1, 2020.

### BACKGROUND

The prevalence of age-related neurodegenerations, including Alzheimer's disease, Parkinson's disease, vascular dementia, amyotrophic lateral sclerosis, Lewy body disease, and frontotemporal dementia are increasing as the aging population grows. Alzheimer's disease is the most common age-related neurodegeneration. The age-corrected incidence of Alzheimer's disease (AD) per 100,000 is increasing. In 2000, the U.S. annual death rate from AD was 17.6 per 100,000, and in 2017, the rate was 37.3 per 100,000. The rate of AD is expected to increase by 3-fold in the next 20 years. However, the baby boomer population bubble does not explain a rate increase, and while improved AD diagnosis may explain some of the increase, it cannot explain it all. Thus, an environmental factor with increasing pervasiveness is likely involved.

Amyloid-β and neurofibrillary tangles begin to appear in the brains of cognitively healthy adults decades before the onset of cognitive impairment. And while PET scans or cerebrospinal fluid biomarkers can detect AD pathology years before symptom onset, these tests are expensive or include a risky lumbar puncture. The blood biomarker p-tau217 can aid in the diagnosis of early AD (Palmqvist et al. 2020). However, it does not identify the non-genetic driver causing the neurodegeneration. A non-genetic driver could be a human herpesvirus infection since herpesviruses have been associated with AD, including human cytomegalovirus (HCMV), the roseoloviruses (HHV-6A/B and HHV-7), human simplex virus 1 and 2 (HSV-1/2), varicella-zoster virus (VZV), Epstein-Barr virus (EBV), and Kaposi's sarcoma-associated herpesvirus (KSHV).

In 1979, Renvoize and colleagues reported finding a significant association between the human leukocyte antigen (HLA) BW15 and AD risk. HCMV antibody titers were significantly higher in AD patients and even higher in patients with BW15 (Renvoize et al. 1979). HLA-BW15 is adjacent to HLA-DR15 in the major histocompatibility region with few recombination hotspots, indicating linkage equilibrium (Bakker et al. 2006). The haplotype HLA-DRB1*15:01 at HLA-DR15 has a significant association with AD risk (Steele et al. 2017). Moreover, HCMV antibody levels are significantly associated with the cortical density of neurofibrillary tangles (Lurain et al. 2013) and AD risk (Barnes et al. 2015).

Peripheral mononuclear blood cells from AD patients have significantly higher interferon-γ levels in response to the HCMV protein pp65 compared to non-demented controls (Westman et al. 2014). In fact, similar to AD patients, HCMV seropositive patients have significantly elevated inflammatory cytokines, such as IL-6 and TNF-α, (Lurain et al. 2013). HCMV DNA was detected by PCR in a significantly higher portion of brains from vascular AD patients than brains from healthy controls (Lin et al. 2002). HCMV DNA may also be present in the brains of AD patients since vascular abnormality is a common AD pathology. However, HCMV DNA positivity was similar in brain samples from AD and healthy controls.

A meta-analysis pooling data from five studies showed that HHV-6 status significantly increased the risk of AD, with a pooled odds ratio of 2.23 (95% CI 0.95-5.33) (Steel and Eslick 2015). Also, AD patients have significantly lower HHV-6 IgG compared to controls, indicating that impaired HHV-6 immunity may increase the risk of AD (Westman et al. 2017). Readhead et al. recently corroborated these findings by finding that AD brains contain significantly more HHV-6A and HHV-7 transcripts compared to brains from cognitive controls, and while HSV-1 transcripts were also more abundant in AD brain, they were much less prevalent than HHV-6A and HHV-7 (Readhead et al. 2018).

Epstein-Barr virus DNA is significantly higher in peripheral blood leukocytes from AD patients than from age-matched control subjects (Carbone et al. 2014). Moreover, acute EBV infection is associated with reduced dendritic cells (Panikkar et al. 2015) and decreased dendritic cell number is associated with AD (Ciaramella et al. 2016). Another finding that supports EBV involvement is clonally expanded T cells found targeting EBV antigens in brain lesions from AD patients (Gate et al. 2020). EBV can also transform B cell lines isolated from AD patients significantly more efficiently than B cells from healthy controls (Ounanian et al. 1992). This finding suggests that a particular EBV entry receptor allotype can increase EBV infection vulnerability and lead to AD. The EBV entry receptors are CD21 (CR2), CD35 (CR1), EphA2, integrin receptors (αvβ5, αvβ6, αvβ8), NRP1, NMHC-IIA, and HLA-DRB1.

Variation in disease expression can result from EBV entry receptor variants and EBV strains with different cell tropism or virulence. Consistent with a role for EBV in AD, CD35, and HLA-DRB1 gene variants are significantly associated with AD risk (Chung et al. 2014; Lu et al. 2017). Notably, individuals with HLA-DRB1 antigen 13 have increased EBV seropositivity (Jabs et al. 1999), while healthy women harboring a single nucleotide variant (HLA-DRB1*13:02), have stable gray matter volume with age compared to women that do not (James et al. 2018).

Endothelial cells express CD21 and CD35 (Timens et al. 1991; Collard et al. 1999). Accordingly, EBV reportedly infects primary human brain microvessel endothelial cells in culture (Casiraghi et al. 2011; Jones et al. 1995). Interestingly, a brain MRI of an acute case of EBV encephalitis showed inflammation restricted to brain microcirculation; and electron microscopy of AD pathology showed microvessel disturbance (Miyakawa 1997; Di Carlo et al. 2011).

Erythrocytes also express CD35 and are anomalous in AD (Kosenko et al. 2017). Furthermore, receiving washed erythrocyte transfusions significantly increases AD risk (Lin et al. 2019). This connection indicates that EBV strains with CD35 tropism may be involved in neurodegeneration. Interestingly, immunolabeling for CD35, in the brain is restricted to astrocytes (Fonseca et al. 2016). However, EBV has been shown to infect neurons (Jha et al. 2015).

EBV is involved in the pathogenesis of multiple sclerosis, and myelin degeneration is the cardinal pathology of multiple sclerosis (Mechelli et al. 2015). Myelin degeneration also occurs in AD, and multiple sclerosis and AD can coexist in patients (Luczynski et al. 2019). Thus, EBV could cause myelin degeneration in AD.

HSV-1 is another herpesvirus implicated in AD pathogenesis. For instance, HSV-1 and -2 infection of neuroblastoma cells replicate AD amyloid-β pathology (Shipley et al. 2005; Chiara et al. 2010). HSV-1 is present in trigeminal ganglion (TG) neurons in an estimated 60-90% of the population, depending upon demographics. TG neurons from AD patients test positive for HSV-1 twice as often as those from cognitive controls (Deatly et al. 1990). HSV-1 is also found in the brainstem of healthy human cadavers (Steiner et al. 1994; Olsson et al. 2016; Hu et al. 2016; Fraser et al. 1981; Baringer and Pisani 1994; Theil et al. 2004). Fraser et al. found 6 of 11 cadavers tested positive for HSV-1 DNA by Southern blot, and Baringer and Pisani found HSV-1 DNA in various brain regions in 14 out of 40 cadavers by PCR, with the pons, medulla, and olfactory bulbs testing positive most often. Theil et al. detected HSV-1 DNA in multiple cranial nerve nuclei from all 5 healthy cadavers tested using PCR but found no LAT RNA expression by RT-PCR. Steiner et al. detected HSV-1 RNA expression in brainstems from all 7 cadavers tested. What is striking about the reports by Steiner and Theil, is that HSV-1 DNA was found in the brainstem of every cadaver tested. Steiner detected HSV-1 transcripts using a larger probe that included LAT, ICP27, U55, and UL56 by in situ hybridization while a smaller probe specific for just LAT was negative on a northern blot. Therefore, LAT may not be expressed by latent HSV-1 in the CNS. However, HSV-2 is found via LAT expression mostly in the brain stem and is responsible for more meningitis cases than HSV-1.

The locus coeruleus (LC) is decimated in AD brains, with a 70% loss of neurons in late-stage disease (Andrés-Benito et. al. 2017). The LC is also one of the first brain regions to show neurofibrillary tangles. HSV infected noradrenergic neurons from the LC is almost certain given that rostral LC neurons are intermingled among the sensory mesencephalic neurons receiving afferent fibers from all three trigeminal nerve branches (André Parent 1996; Geerling et al. 2010). The trigeminal neurons innervate widely throughout the mesencephalic nucleus to transmit pressure and kinesthesis sensory from the teeth, hard palate, joint capsule, periodontium, lingual, facial, and extraocular muscles (Andre Parent 1996; Young and Perryman 1984). Furthermore, motoneurons residing in the trigeminal motor nucleus send fibers directly through the mandibular branch of the trigeminal nerve, passing underneath the ganglion to innervate jaw muscles. Neurons of the mesencephalic nucleus are also positioned directly rostral to the main LC nucleus which has proximity to the LC region with the most severe degeneration. In addition to entering the CNS at the mesencephalic nucleus, HSV can travel anterograde in axons from bipolar trigeminal neurons to the three trigeminal nerve nuclei (mesencephalic, sensory, and spinal). HSV can also take a direct path to the CNS through the olfactory nerve and optic nerve. In summary, it is not surprising that HSV can infect the CNS in healthy immunocompetent humans given the direct pathways to the CNS.

Noradrenergic neurons in the LC have a direct pathway to the hippocampus. LC noradrenergic neurons innervate hippocampal microvessels releasing the vasopressor norepinephrine. Hyperactivity of HSV infected LC noradrenergic neurons could increase vasoconstriction resulting in hypoxia. Interestingly, live imaging of mouse autonomic nuclei infected with a type of alphaherpesvirus showed groups of neurons with aberrant synchronous firing (Granstedt et al. 2013).

VZV is also found in human TG neurons. However, VZV is not found consistently increased in AD brains compared to brains from healthy controls (Warren-Gash et al. 2019). Although, the inconsistency could be related to regions sampled and methodology. VZV can cause vasculopathy in both small and large blood vessels which increases the risk of stroke and is associated with giant cell arteritis (Nagel et al. 2015). Giant cell arteritis is associated with herpes zoster ophthalmicus, which incidentally significantly increases the risk of developing dementia within 5 years (Tsai et al. 2017). In summary, occult reactivation of VZV can lead to undetected vasculopathy that leads to stroke and dementia.

Recently, KSHV DNA was the highest herpesvirus DNA in community wastewater (Miyani et al. 2020). In the United States, KSHV infection prevalence is thought to be between 6-10%, the lowest of all herpesviruses, the high KSHV genome copy number was explained by high HIV positivity in the community at the time of measurement. However, KSHV seroprevalence data dates to the late 1990's when assay results were inconsistent and Kaposi's sarcoma patients themselves could have 70-90% seropositivity depending on the assay. Also, KSHV seropositivity can subsequently show seroreversion (Martin J N 2007). The KSHV human IL-6 homolog vIL-6 is detected in the serum of about 1 in 20 healthy blood donors in the United States and about 1 in 5 donors from Italy (Aoki et al. 2001). vIL-6 is produced predominantly in lytic infection (Chen and Lagunoff 2007); therefore, approximately one in 20 healthy adults in the United States may have subacute lytic KSHV infection. A high KSHV seropositivity rate of 26% was found in south Texas children (Baillargeon et al. 2002). In contrast, no KSHV DNA was not detected in blood samples by quantitative polymerase chain reaction (PCR) from 684 blood donors (Qu et al. 2010). The prevalence of Kaposi's sarcoma virus in the United States today is truly unknown and it is apparent that KSHV infection prevalence is highly variable geographically—as is AD (Russ et al. 2012). Interestingly, KSHV has a high prevalence in Italy and sub-Saharan African, and the middle east, and these geographical locations also show the highest AD incidence (Mubangizi et al. 2020; Cesarman et al. 2019). Like the other gamma herpesvirus EBV, Kaposi's sarcoma virus infects immune cells, endothelial cells, and neurons (Tso et al. 2017).

Although evidence suggests an association between infection with multiple herpesvirus types and AD there are also credible conflicting reports (Allnutt et al. 2019; Jeong and Liu 2019; Warren-Gash et al. 2019; Nath 2018). As a result, most AD researchers are unconvinced that herpesviruses are involved in AD or other age-related neurodegeneration, and only a few researchers are examining a herpesvirus causation for AD.

### SUMMARY

The invention described herein is a method of diagnosing and quantifying herpesvirus-mediated neurodegeneration comprised of measuring herpesvirus secretome proteins and RNA in blood or a blood component from an individual. The herpesvirus secreted protein and RNA levels are compared to a baseline reference level to assess herpesvirus activity level. In some cases, the human cytomegalovirus secreted proteins are measured, the proteins selected from pp52 (SEQ ID NO 1), IE1 (SEQ ID NO 2), IE2 (SEQ ID NO 3), pp150 (SEQ ID NO 4), UL21.5 (SEQ ID NO 5), UL7 (SEQ ID NO 6), UL128 (SEQ ID NO 7), vCXCL-1 (SEQ ID NO 8), vCXCL-2 (SEQ ID NO 9), vIL-10 (SEQ ID NO 10), LAvIL-10 (SEQ ID NO 11), early nuclear protein HWLF1 (SEQ ID NO 12), US34 (SEQ ID NO 13), and UL13 (SEQ ID NO 14). In some cases, the human herpesvirus 6A secreted proteins are measured, the proteins selected from pp100 (SEQ ID NO 15), IE1 (SEQ ID NO 18), IE2 (SEQ ID NO 19), PPS (SEQ ID NO 20), glycoprotein B (SEQ ID NO 21) and DR1/DR2 (SEQ ID NO 22). In some cases, the human herpesvirus 6B protein U83 (SEQ ID NO 23) and/or glycoprotein B (SEQ ID NO 24) is measured. In some cases, the human herpesvirus 7 secreted proteins are measured, the proteins selected from pp100 (SEQ ID NO 16), IE1 (SEQ ID NO 25), IE2 (SEQ ID NO 26), PPS (SEQ ID NO 27), glycoprotein B (SEQ ID NO 28), and DR1/DR2 (SEQ ID NO 29). In some cases, Epstein-Barr virus secreted RNAs are measured, the RNAs selected from EBER1 (SEQ ID NO 31), EBER2 (SEQ ID NO 32), BHRF1-1 (SEQ ID NO 33), BHRF1-2 (SEQ ID NO 34), BHRF1-3 (SEQ ID NO 35), EBV-mir-BART1 (SEQ ID NO 36), EBV-mir-BART2 (SEQ ID NO 37), EBV-mir-BART3 (SEQ ID NO 38), EBV-mir-BART4 (SEQ ID NO 39), EBV-mir-BART5 (SEQ ID NO 40), EBV-mir-BART6 (SEQ ID NO 41), EBV-mir-BART7 (SEQ ID NO 42), EBV-mir-BART8 (SEQ ID NO 43), EBV-mir-BART9 (SEQ ID NO 44), EBV-mir-BART10 (SEQ ID NO 45), EBV-mir-BART11 (SEQ ID NO 46), EBV-mir-BART12 (SEQ ID NO 47), EBV-mir-BART13 (SEQ ID NO 48). In some cases, the human proteins associated with Epstein-Barr virus RNA and Epstein-Barr virus secreted proteins are measured, the proteins selected from gp350 (SEQ ID NO 17), Homo Sapiens La (SEQ ID NO 49), Homo Sapiens L22 (SEQ ID NO 50), LMP1 (SEQ ID NO 51), LMP2A (SEQ ID NO 52), and ebvIL-10 (SEQ ID NO 53). In some cases, the human simplex virus type 1 secreted proteins are measured, the proteins selected from VP22 protein (SEQ ID NO 54), glycoprotein B (SEQ ID NO 55), glycoprotein C (SEQ ID NO 56), and UL56 (SEQ ID NO 57). In some cases, the human simplex virus type 2 secreted proteins are measured, the proteins selected from glycoprotein B (SEQ ID NO 58), glycoprotein G (SEQ ID NO 59), and peptide gG-2p20 (SEQ ID NO 60). In some cases, the VZV secreted proteins are measured, the proteins selected from glycoprotein B (SEQ ID NO 61), glycoprotein E (SEQ ID NO 62), glycoprotein I (SEQ ID NO 63), glycoprotein C (SEQ ID NO 64), glycoprotein H (SEQ ID NO 65), and glycoprotein L (SEQ ID NO 66). In some cases, proteins secreted from each herpesvirus type are measured in a screening assay to identify the active herpesviruses. In some cases, Kaposi's sarcoma virus secreted proteins and RNA are measured, the proteins selected from vIL-6 (SEQ ID NO 67), ORF8.1 (SEQ ID NO 68), ORF4 (SEQ ID NO 69); the RNA selected from KSHV-miR-K12-1 (SEQ ID NO 70), KSHV-miR-K12-2 (SEQ ID NO 71), KSHV-miR-K12-3 (SEQ ID NO 72), KSHV-miR-K12-4 (SEQ ID NO 73), KSHV-miR-K12-5 (SEQ ID NO 74), KSHV-miR-K12-6 (SEQ ID NO 75), KSHV-miR-K12-7 (SEQ ID NO 76), KSHV-miR-K12-8 (SEQ ID NO 77), KSHV-miR-K12-9 (SEQ ID NO 78), KSHV-miR-K12-10 (SEQ ID NO 79), KSHV-miR-K12-11 (SEQ ID NO 80), KSHV-miR-K12-12 (SEQ ID NO 81).

In some embodiments, herpesvirus secretome information is combined with blood Alzheimer's disease biomarkers to indicate disease stage and progression rate. In some cases, the Alzheimer's disease biomarker measured is a coagulation factor, the molecules selected from γ-chain-fibrinogen (SEQ ID NO 82), thrombin (SEQ ID NO 83), tissue thromboplastin (SEQ ID NO 84), plasminogen activator inhibitor type 1 (SEQ ID NO 85), D-dimer (SEQ ID NO 86), Von Willebrand factor (SEQ ID NO 87) Bradykinin (SEQ ID NO 88), antithrombin (SEQ ID NO 89), activated factor XII (SEQ ID NO 90), activated factor VIII (SEQ ID NO 91), Kallikrein-8 (SEQ ID NO 92), activated factor VII (SEQ ID NO 93), and activated factor X (SEQ ID NO 94). In some cases, the Alzheimer's disease biomarker measured is a cellular adhesion molecule, the molecules selected from VCAM-1 (SEQ ID NO 95), ICAM-1 (SEQ ID NO 96), and E-Selectin (SEQ ID NO 97). In some cases, the Alzheimer's disease biomarker measured is selected from the soluble HLA-G1 protein (SEQ ID NO 30), amyloid-β (SEQ ID NO 98), alpha1-microglobulin (SEQ ID NO 99), complement factor I (SEQ ID NO 100), complement 3a (SEQ ID NO 101), and noradrenaline. In some embodiments, coagulation factor, cell adhesion molecule, and Alzheimer's disease biomarkers levels are combined with herpesvirus secretome proteins or RNA levels, to improve the accuracy of detecting AD neuropathology, estimating disease stage, and predicting progression rate. In some cases, the secretome proteins or RNA, coagulation factors, cell adhesion molecules, and Alzheimer's disease biomarkers are measured by an enzyme-linked immunosorbent assay.

In some embodiments, the enzyme-linked immunosorbent assay is utilized in a diagnostic kit for measuring the herpesvirus secretome factors in blood, wherein the kit comprises: (a) an antibody or aptamer which specifically binds to a herpesvirus secretome protein or RNA; and (b) a solid matrix to which the antibody or aptamer is bound; and (c) reagents for detecting a complex between the antibody or aptamer and a sample. In some cases, a screening kit includes an assortment of antibodies or aptamers that target secretome proteins or RNA from the eight herpesviruses as listed above. In some cases, the kit includes antibodies or aptamers that target secreted factors from one herpesvirus type. In some cases, the kit includes an antibody or aptamer that targets secreted herpesvirus factors and a coagulation factor, cellular adhesion molecule, and/or Alzheimer's disease biomarker.

In some cases, the identity and quantity of herpesvirus secreted proteins and RNA are measured in blood using mass spectrometry. In some cases, a patient's blood is dried on paper and mailed to a processing center for testing. The described invention is the first sensitive and economical method to detect herpesvirus activity in blood and thereby quantify the extent and progression rate of herpesvirus-mediated neurodegeneration.

### DETAILED DESCRIPTION

Herpesviruses, especially HSV-1, have been suspected to be involved in AD for at least four decades (Middleton P J et. al. 1980). Many reports refute an association between herpesvirus types and AD. (Deatly et al. 1990; Kittur et al. 1992; Allnutt et al. 2019; Hemling et al. 2003; Jeong and Liu 2019). After decades of conflicting reports, the question remains an enigma. The absence of herpesvirus DNA in AD brains can be explained in part by when and where the tissue has been sampled. First, most AD brain samples are taken postmortem from late-stage AD patients. This is analogous to examining the cause of an auto accident after the drivers and wreckage have long left the scene. Herpesviruses can infect blood vessels, including pericytes, endothelial cells, and astrocytes, and some microvessels are obliterated in some AD brain regions (Miyakawa 1997). Moreover, significant pathology occurs in the brain stem, and this region is rarely sampled. Another reason for conflicting reports is that herpesviruses have evolved mechanisms to restrict spreading to avoid immune detection and the triggering a fatal inflammatory response like encephalitis. Accordingly, brains from transplant patients with HCMV disease show widespread neurodegeneration but only sparsely infected cells. Thus, substantial herpesvirus-mediated AD neuropathology is non-cell-autonomous, and caused by secreted factors from few infected cells (Kosugi et al. 1998; Bigger et al. 2000; Zhang and Atherton 2002). The reactivation or infection of few cells makes it difficult to detect viral RNA/DNA and conclude disease association. Furthermore, scarce RNA detection may be beyond detection limits of the reverse transcriptase reaction in the presence of significant background RNA (Levesque-Sergerie et al. 2007).

Human herpesviruses remain latent in most cells throughout an individual's life and reactivate only in replication reservoirs of the oropharynx and urogenital regions for transmission. With increasing age epigenetic dedifferentiation lowers herpesvirus reactivity threshold (Lupey-Green et al. 2018), increasing reactivation frequency (Parry et al. 2016; Stowe et al. 2007). During times of infection, inflammation, and stress, latent herpesviruses reactivate in peripheral blood mononuclear cells. In an aged person, reactivated herpesviruses may not be subdued as readily by immunity and epigenomic silencing, resulting in chronic occult activity. With increased reactivation frequency, the virus spreads throughout the body increasing viral load. This scenario explains why initiating events, such as infection, inflammation, or stress are risk factors for developing AD in the elderly (Phelan et al. 2012; Ehlenbach et al. 2010; Norton et al. 2010).

Herpesvirus infected peripheral blood mononuclear cells migrate to areas releasing chemokines. Once there, inflammatory cytokines induce terminal differentiation of peripheral blood mononuclear cells, including monocyte-derived macrophages and dendritic cells. Terminal differentiation induces herpesvirus reactivation and replication. The cells forming the neurovascular unit, including endothelial cells, pericytes, smooth muscle cells, and astrocytes can become infected by HSV-1 and -2, VZV, EBV, HCMV, HHV-6A/B and HHV-7, and KSHV. Herpesviruses secrete viral factors during latency and replication which can induce inflammation, spread immunosuppression, and alter the physiology of uninfected cells (Mason et al. 2012). Many of these secreted viral factors can be detected in peripheral blood (Young 2015; Teow et al. 2017; Taga et al. 1995; Aoki et al. 2001; Polizzotto et al. 2013). Herpesvirus secretomes can also be detected in saliva or urogenital secretions, and while there is strain resemblance with blood components, there is also evidence of discordance, indicating that distinct strains infect anatomical sites (Kwok et al. 2015). Moreover, the secretome in replication reservoirs could reflect intermittent shedding and not disease.

Chemokine release can attract multiple herpesvirus types to an infection site and localized immunosuppression can increase the reactivation, exacerbating inflammation further. Likewise, encephalitis and transplant surgery fatalities increase with the number of herpesvirus types reactivated (Sánchez-Ponce et al. 2018; Tang 1997). And transplant patients with poor outcomes often have herpesvirus coinfections (Sánchez-Ponce et al. 2018). Furthermore, cytomegalovirus-mediated immune modifications appear to upregulate a secondary inflammatory response. For example, murine cytomegalovirus (MCMV) infection produced an enhanced immune response in experimental autoimmune encephalomyelitis (EAE), a mouse model of multiple sclerosis (Vanheusden et al. 2017). The increased immune response may be related to CD4+CD28^{null} T cell expansion resulting from repeated MCMV antigen stimulation. In EAE, the mouse is immunized with myelin oligodendrocyte glycoprotein (MOG) to induce an immune response. The MCMV/EAE mouse is a model for human HCMV and EBV coinfection. This is not restricted to HCMV since HSV-1 infection of the Akata B-cell line reactivates a latent EBV infection (Wu et al. 2012). Thus, humans infected with multiple herpesviruses, such as HSV-1 and -2, VZV, HCMV, and EBV are more likely to reactivate latent herpesviruses and have increased neuroinflammation leading to AD.

A multiplex PCR amplifying all nine human herpesvirus in multiple human fluids and tissues from 60 individuals found that most individuals were infected with multiple herpesviruses (Pyöriä et al. 2020). Interestingly, Pyöriä did not use whole blood to detect herpesvirus DNA which typically yields much higher viral load. Given the evidence that most people are infected with multiple herpesvirus types, and that herpesviruses reactivate with age, different types of neurodegenerations, i.e., AD, Parkinson's disease, multiple sclerosis, vascular dementia, amyotrophic lateral sclerosis, Lewy body disease, and frontotemporal dementia, could be caused by different combinations of herpesvirus types and strains. Indeed, transcriptome analyses from AD patients revealed two different subtypes of AD (Milind et al. 2020). The number of herpesvirus types and strains, age at primary infection, viral load, host genetics, diet, and stress are likely to define the age of dementia symptom onset and progression rate.

Herpesvirus activity is currently evaluated by immunoglobin levels and viral RNA/DNA (Ross 2011). HCMV is also evaluated by the pp65 antigenemia assay. However, each of these methods has disadvantages. Immunoglobin measures previous exposure and are a poor measure of chronic subclinical activity (Li et al. 2014; Cone et al. 1993). Moreover, seropositivity is not an accurate indicator of HCMV infection. One study showed half of the healthy HCMV⁺ DNA positive and vIL-10⁺ donors were HCMV seronegative (Young et al. 2017). The presence of viral RNA/DNA in plasma can be transient and missed in a routine screening (Lodding et al. 2018). Moreover, blood viral RNA/DNA does not reflect herpesvirus activity at anatomical sites (Kwok et al. 2015). Also, the presence of viral RNA/DNA in plasma or serum is not reliable because it does not distinguish disease versus intermittent shedding. The cost for reagents and labor in screening RNA from multiple herpesviruses by reverse transcription and PCR amplification is cost-prohibitive. Also, the HCMV pp65 antigenemia is a labor-intensive assay. Furthermore, these methods do not reveal latent viral activity or activity involving the release of capsid types A and B, or dense bodies, which contain hundreds proteins and can produce significant neuropathology without viral DNA. Lastly, measuring roseolovirus DNA viral load is especially problematic because some individuals inherit the virus chromosomally integrated.

Latent and lytic herpesviruses secrete herpesvirus factors that can alter neighboring cell physiology, increase angiogenesis, and alter immunity. Immunosuppression caused by herpesvirus secreted factors can increase susceptibility to opportunistic infections (Poole and Sinclair 2015). Measuring the viral secretome in blood is a sensitive indicator of total herpesvirus viral load and has the potential to distinguish lytic and latent activity. The method of identifying and quantifying herpesvirus activity by measuring secreted herpesvirus factors in the blood can aid in the early diagnosis of neurodegeneration.

Currently, herpes zoster ophthalmicus is often associated with alphaherpesvirus-mediated giant cell arteritis, including HSV-1 and -2 and VZV (Powers et al. 2005; England et al. 2017). Occult HCMV infection can also cause retinitis in immunocompetent individuals (Karkhaneh et al. 2016; Stewart et al. 2005). Notably, both can be problematic to diagnose, and yet urgent diagnosis is critical to prevent permanent vision loss. The detection of secreted herpesvirus factors in the blood of patients with acute herpesvirus-mediated vision loss is a quick, precise, and low-cost diagnostic tool. Furthermore, AD is associated with retinal pathology, and thus retinal pathology combined with the detection of secreted herpesvirus can aid in the diagnosis of AD (Koronyo et al. 2017). In summary, the characterization of herpesvirus secretomes has the potential to be the earliest and most sensitive biomarker for predicting herpesvirus-mediated neurodegeneration.

The function of secreted viral proteins in uninfected cells can be presumed based on their known function in infected cells. For example, within HCMV infected cells, pp150 (SEQ ID NO 4) binds to the protein bicaudal D1 (BicD1). BicD1 binds Rab6 vesicles to the dynein-dynactin complex for intracellular movement along microtubules. Binding of pp150 to BicD1 is required to translocate Rab6 vesicles to the viral assembly compartment. Rab6 also binds the spliced isoform APP-binding family A member 1 (APBA), previously called Mint1 826, which colocalizes with amyloid precursor protein (APP) during vesicle transport. The displacement of Rab6 vesicles to the viral assembly compartment by pp150 must also disrupt APP trafficking. In uninfected cells without an assembly compartment, secreted pp150 could enter uninfected cells, bind BidD1, and disrupt normal Rab6|Mint826|APP trafficking.

MCMV infected murine fibroblasts have increased APP, total tau, and serine 396 phosphorylated tau expression but do not produce more amyloid-β. However, how MCMV infection affects APP processing may differ between neurons and other cells, such as astrocytes, and between infected cells and uninfected cells. Accordingly, many defined regions of tau deposits do not overlap amyloid-β deposits in AD brains (Uematsu et al. 2018). The displacement of Rab6 vesicles to the viral assembly compartment could affect neurite maintenance since the restriction of BicD1|Rab6 to the centrosome prevents anterograde transport, which inhibits neuritogenesis (Schlager et al. 2010). Rab6 may also be involved in transporting late endosomes to the lysosome (Patwardhan et al. 2017). Thus, pp150-mediated Rab6 vesicle displacement effects are consistent with some AD pathology. Disseminated pp150 could cause widespread non-cell-autonomous degeneration with few infected cells. Detection of pp150 in blood could be a direct measure for HCMV-mediated neurodegeneration. Increasing pp150 levels are independent of regenerative capacity and could be identified much earlier than the current biomarkers amyloid-β and p-tau.

Homologs to HCMV pp150 are found in the roseolovirus (HHV6A and HHV7) and EBV. Roseolovirus sequence similarity is at the N terminal of pp150 (SEQ ID NO 4), with HHV6A pp100 (SEQ ID NO 15) having 45% positives and an E value of 1×10⁻²⁰ and HHV7 pp100 (SEQ ID NO 16) having 42% positives and an E value of 1×10⁻¹⁶. EBV glycoprotein 350 (gp350) has a shorter sequence similarity located toward the carboxyl-terminal of the protein with 49% positives and an E value of 6×10⁻⁰⁵. Also, gp350 (SEQ ID NO 17) has sequence alignment to HCMV pp150 where pp150 binds BicD1. Thus, pp100 from HHV6A and HHV7, and EBV gp350 could also bind BicD1 to prevent normal Rab6-APP vesicles trafficking.

In one embodiment, secreted factors from latent and lytic herpesvirus are measured for detecting which herpesviruses are present and for staging herpesvirus-neuropathology and progression rate. In this respect, neuropathology or neurodegeneration includes herpesvirus-mediated retinal disease. The viral secretome can be measured in serum or plasma and can be contained within exosomes. The level of herpesvirus secretome proteins or RNAs over a background control or a baseline reference value is indicative of herpesvirus-mediated neurodegeneration. In some embodiments, HCMV secreted proteins are measured in blood from individuals to identify and quantify their HCMV-mediated neuropathology, the proteins selected from: (1) the phosphoprotein pp52 (SEQ ID NO 1) encoded by the UL44, and also known as ICP36 or DNA polymerase processivity subunit (PPS); (2&3) the viral transcription factors IE1 (SEQ ID NO 2) and IE2 (SEQ ID NO 3); (4) the tegument phosphoprotein pp150 (SEQ ID NO 4); (5) the glycoprotein homolog of cellular RANTES, pUL21.5 (SEQ ID NO 5); (6) the novel glycoprotein UL7 (SEQ ID NO 6), shown to inhibit inflammatory signaling and increase tubulogenesis; (7) the chemokine like protein UL128 (SEQ ID NO 7) that functions like CCL3/MIP-1 a by attracting PBMCs; (8&9) viral CXC chemokines, vCXCL-1 (SEQ ID NO 8) and vCXCL-2 (SEQ ID NO 9; (10) a homolog of human IL-10, vIL-10 (SEQ ID NO 10), functions to suppress immunity; (11) a homolog of human IL-10 expressed during latency, LAvIL-10 (SEQ ID NO 11); (12) early nuclear protein HWLF1 (SEQ ID NO 12); (13) protein US34 (SEQ ID NO 13); (14) protein UL13 (SEQ ID NO 14) (Dumortier et al. 2008; Luganini et al. 2016; Mocarski et al. 1988; Arvin A, Campadelli-Fiume G, Mocarski E, et al 2007).

The HCMV gene UL111A is alternatively spliced during latency resulting in a premature stop codon in intron 2 that produces a 139 amino acid protein. The full-length vIL-10 protein (SEQ ID NO 10) is 175 amino acids long. A polyclonal goat antibody to vIL-10 from R&D Systems (AF117) detects both vIL-10 and LAvIL-10, as shown by Western (Young 2015; Young et al. 2017). LAvIL-10 and vIL-10 are detected in serum from healthy HCMV seropositive donors using sandwich ELISA. FIG. 1 shows the wide range of vIL-10 levels among donors. Moreover, vIL-10 levels appear to increase in females and a decrease in males. Used with permission from author and copyright owner Vivian P. Young.

In some embodiments, roseolovirus secreted proteins are measured in blood from individuals to identify and quantify roseolovirus herpesvirus-mediated neuropathology, the HHV-6A proteins selected from pp100 (SEQ ID NO 15), IE1 (SEQ ID NO 18), IE2 (SEQ ID NO 19), PPS (SEQ ID NO 20), glycoprotein B (SEQ ID NO 21), and DR1/DR2 (SEQ ID NO 22); the HHV-6B proteins selected from U83 (SEQ ID NO 23) and glycoprotein B (SEQ ID NO 24); and HHV-7 proteins selected from pp100 (SEQ ID NO 16), IE1 (SEQ ID NO 25), IE2 (SEQ ID NO 26), PPS (SEQ ID NO 27), glycoprotein B (SEQ ID NO 28), and DR1/DR2 (SEQ ID NO 29).

The HHV6 gene U94 is found only in HHV6 and encodes a protein called RepH6. RepH6 expression is required for latent infection but is also expressed during productive infection (Rotola et al. 1998). RepH6 increases the expression through the upregulation of the transcription factor ATF3 (Rizzo et al. 2018). Increased ATF3 expression is also induced by lipolysis products and hippocampal endothelium is particularly sensitive to ATF3 upregulation. Increased ATF3 expression is associated with increased expression of proinflammatory response and oxidative stress genes (Aung et al. 2016). Soluble human leukocyte antigen G1 (sHLA-G1) inhibits angiogenesis by binding directly to the CD160 receptors on endothelial cells to induce apoptosis (Fons et al. 2006; Le Bouteiller et al. 2007). Wiendl and colleagues found increased HLA-G1 expression in brain tissue from two AD patients compared to healthy controls (Wiendl et al. 2005). Serum sHLA-G1 could be an additional sensitive early biomarker for herpesvirus-mediated AD pathogenesis (Wiendl et al. 2005). In some cases, an individual's herpesvirus secretome levels are combined with the level of human sHLA-G1 (SEQ ID NO 30) to determine neurodegeneration risk and stage.

EBV expresses two non-coding RNAs, EBER1 (SEQ ID NO 31) and EBER2 (SEQ ID NO 32). The EBERs are expressed at high levels during latent and lytic infection. EBER1 was found in blood bound to the human lupus-associated (La) protein (Iwakiri et al. 2009). EBER1 was also found bound to human L22 ribosomal protein in uninfected cells (Toczyski et al. 1994). EBER1 can bind and inactivate the protein kinase R (PKR), preventing viral RNA activated PKR mediated protein synthesis arrest. EBER1 concentration is associated with the expression interferon related genes and IL-6 (Aromseree et al. 2017). Individuals afflicted with an EBV-associated disease have significantly higher dementia risk (Hou et al. 2019; Luczynski et al. 2019; Chen et al. 2019; Zhao et al. 2018). EBER2 secretion has not been reported but secretion could depend on cell type.

Neurons from AD brains show abnormal expression of cell-cycle entry proteins cyclins-D and —B and increased hyperploidy. Cell cycle entry is abnormal for postmitotic neurons and is linked to synaptic dysfunction and neuron death. EBV expresses high levels of two short ncRNA molecules, EBER1 and EBER2, during latency and replication. EBER expression induces IL-6 mediated activation of signal transducers and activators of transcription 3 (STAT3). STAT3 activation decreases the expression of cyclin dependent kinase inhibitor p21 and p27, which releases cyclin dependent kinases 2 and 4 inhibition, allowing cyclins to promote G1/S transition.131,132 Cyclin-dependent kinase 4 activation induces cell death by hyperphosphorylation of the pRb family member p130. Phosphorylated p130 binds chromatin modifiers Suv39H1 and HDAC1, releasing the transcription factor E2F4, which binds transcription factors B and C-Myb to initiate transcription of the proapoptotic B113-containing BIM. BIM activates BAX/BAK, which forms multimeric pores in the mitochondrial membrane to release cytochrome c. Cytochrome c binds the protein 14-3-3ε to release its inhibition over the apoptotic protease activating factor-1, allowing apoptosome formation and apoptosis-inducing caspase 9/3 activation.133 Accordingly, EBER1 could be responsible for the abnormal expression of cell cycle entry proteins and increased BIM expression in the AD brain.

EBER1 can bind to TLR3 on neurons to cause irreversible growth cone collapse and inhibit neurite outgrowth. Exosomes containing EBERs are released from nasopharyngeal tumors and delivered to adjacent endothelial cells where EBERs upregulated angiogenesis. Interestingly, vascular density is increased in AD hippocampus compared to controls. Thus, secreted EBER could cause non-cell autonomous increase in growth cone collapse, angiogenesis, and reduced antiviral defense. Interestingly, granulomas in EBV-mediated nasopharyngeal cancers exhibit cells with intracellular amyloid deposits that do not correspond to the EBER expressing epithelial cells. However, EBV also secretes latent proteins LMP1 and LMP2A in exosomes and LMP1 is associated with CD63 in endosomes.

EBV also secretes duplex mature miRNAs in exosomes during latent and active infection (Teow et al. 2017; Pfeffer et al. 2004). Three EBV-miRNAs are located within the BHRF1 gene (cluster 1) and 13 EBV-miRNAs are located within the BART gene (cluster 2). The copy number of these miRNAs can be detected in uninfected peripheral blood mononuclear cells by multiplex quantitative real-time PCR of the reverse-transcribed RNA (Pegtel et al. 2010). Some EBV-miRNAs function to alter host immunity. In some embodiments, secreted RNA is measured in blood or isolated peripheral blood mononuclear cells from an individual to identify and quantify EBV-mediated neuropathology, the RNA selected from EBER1 (SEQ ID NO 31), EBER2 (SEQ ID NO 32), EBV-mir-BHRF1-1 (SEQ ID NO 33), EBV-mir-BHRF1-2 (SEQ ID NO 34), EBV-mir-BHRF1-3 (SEQ ID NO 35), EBV-mir-BART1 (SEQ ID NO 36), EBV-mir-BART2 (SEQ ID NO 37), EBV-mir-BART3 (SEQ ID NO 38), EBV-mir-BART4 (SEQ ID NO 39), EBV-mir-BART5 (SEQ ID NO 40), EBV-mir-BART6 (SEQ ID NO 41) EBV-mir-BART7 (SEQ ID NO 42), EBV-mir-BART8 (SEQ ID NO 43), EBV-mir-BART9 (SEQ ID NO 44), EBV-mir-BART10 (SEQ ID NO 45) EBV-mir-BART11 (SEQ ID NO 46), EBV-mir-BART12 (SEQ ID NO 47), EBV-mir-BART13 (SEQ ID NO 48). Although a single strand miRNA sequence is listed, the disclosure is presumed includes the complementary sequence as well.

During lytic infection, EBV secretes a homolog of human IL-10, ebvIL-10. The ebvIL-10 protein shares 70% amino acid sequence with human IL-10, and absent a few costimulatory effects, has similar immunosuppressive effects. A mouse monoclonal antibody from R&D Systems (MAB9151) can distinguish ebvIL-10 from human IL-10. EbvIL-10 is detected in the serum or plasma from about 20% of infectious mononucleosis patients (Taga et al. 1995). In some embodiments, human proteins associated with EBV RNAs or EBV secreted proteins are measured in blood from an individual to identify and quantify EBV-mediated neuropathology, the proteins selected from Homo Sapien La (SEQ ID NO 49) and Homo Sapien L22 (SEQ ID NO 50), LMP1 (SEQ ID NO 51) and LMP2A (SEQ ID NO 52), ebvIL-10 (SEQ ID NO 53), and gp350 (SEQ ID NO 17).

HSV infected neuroblastoma cells increase amyloidogenic APP processing (Chiara et al. 2010; Shipley et al. 2005). HIV infected macrophages and microglia also misprocess APP to produce toxic amyloid-β. In HIV infected cells, APP associates with the HIV Gag polyprotein, restricting HIV particles from translocating to lipid rafts, but Gag induces APP secretase cleavage for release, resulting in amyloidogenic processing (Chai et al. 2017; Hategan et al. 2019). HSV-1 appears to use a similar strategy as HIV to reduce APP. The HSV-1 glycoprotein B (gB) contains an N-terminal sequence with 67% sequence similarity to the transmembrane region of APP (Cribbs et al. 2000). APP transmembrane sequence associates and is cleaved by the beneficial α-secretase (ADAM10). Thus, HSV-1 gB can theoretically compete with α-secretase for APP binding, effectively reducing APP α-secretase cleavage into the beneficial uAPP-C-terminal fragment; and thus, promoting β- and γ-secretase APP cleavage to produce amyloid-β. The N-terminal of gB has a signal peptide cleavage site, which would produce a 30-amino acids cleavage product, gB1. It is also possible that α-secretase cleaves gB into gB1. The gB1 peptide is secreted by HSV infected cells in culture and is neurotoxic (Pachl et al. 1987). Neurons cultured with the gB1 fragment for 24 hours show neurite retraction, neuritic dystrophy, reduced cell size and increased death (Cribbs et al. 2000).

The HSV-1 protein VP22 is secreted and taken up by uninfected neighboring cells (Elliott and O'Hare 1997). VP22 may have a binding partner, such as glycoprotein E. VP22 associates with Rab6 vesicles and may function like HCMV pp150 in disrupting APP|Rab6 trafficking. VP22 inhibits the cell's ability to detect viral DNA by inactivating cyclic GMP/AMP synthase. Thus, disseminated VP22 can increase susceptibility to viral infection. Measuring VP22 in blood can indicate HSV-1 activity and thus is a predictive biomarker for HSV-i-mediated neurodegeneration. The HSV-1 glycoprotein C is secreted from infected cells (Sedlackova et al. 2008). In some embodiments, HSV-1 secreted proteins is measured in blood from an individual to identify and quantify HSV-1-mediated neuropathology, the proteins selected from VP22 (SEQ ID NO 54), glycoprotein B (SEQ ID NO 55), glycoprotein C (SEQ ID NO 56), and UL56 (SEQ ID NO 57) or cleaved peptides.

Although VP22 shares 65% of the amino acid sequence between HSV-1 and HSV-2, HSV-2 VP22 does not contain a cell-penetrating peptide domain. HSV-2 glycoprotein G is secreted as a 34 kDa high mannose cleavage product (SgG2) corresponding to the N-terminal 300 amino acids of glycoprotein G. SgG2 is further processed into a 15 amino acid peptide (gG-2p20). The peptide gG-2p20 produces monocyte and neutrophil chemotaxis and neutrophil oxidative burst that can kill lymphocytes and NK cells (Bellner et al. 2005). Thus, the gG-2p20 peptide is proinflammatory and could produce non-cell-autonomous degeneration and immunosuppression. In some embodiments, HSV-2 secreted proteins are measured in blood from an individual to identify and quantify HSV-2-mediated neuropathology, the proteins selected from HSV-2 glycoprotein B (SEQ ID NO 58), glycoprotein G (SEQ ID NO 59), and cleaved peptide gG-2p20 (SEQ ID NO 60).

In some embodiments, the VZV secreted proteins or their cleaved peptides, are measured in blood to detect and quantify VZV-mediated neuropathology, the proteins selected from glycoprotein B (SEQ ID NO 61), glycoprotein E (SEQ ID NO 62), glycoprotein I (SEQ ID NO 63), glycoprotein C (SEQ ID NO 64), glycoprotein H (SEQ ID NO 65) and glycoprotein L (SEQ ID NO 66).

KSHV has multiple strain types that have selective cell tropism. For example, a strain type could specifically target lymphatic or microvessel endothelial cells and induce inflammation. Chronic inflammation can be stimulated by the cytokine human interleukin 6 (hIL-6). KSHV secretes a homolog of hIL-6, viral IL-6 (Uldrick et al. 2010). KSHV infected cells also express viral ORF 8.1, a 197 amino-acid protein containing a signal sequence without a transmembrane domain which is likely to be secreted (Chandran et al. 1998). During latency, KSHV exports 12 duplex mature miRNAs in exosomes (Yogev et al. 2017). These miRNAs change the metabolism of nearby cells to support the viral growth, including reduced mitochondrial biogenesis, aerobic glycolysis, and antiviral response (Top et al. 2016). In some embodiments, the KSHV secreted proteins or miRNA, are measured in blood to detect and quantify KSHV-mediated neuropathology, the proteins selected from vIL-6 (SEQ ID NO 67), ORF 8.1 (SEQ ID NO 68), ORF4 (SEQ ID NO 69), and miRNAs selected from, miR-K12-1 (SEQ ID NO 70), miR-K12-2 (SEQ ID NO 71), miR-K12-3 (SEQ ID NO 72), miR-K12-4 (SEQ ID NO 73), miR-K12-5 (SEQ ID NO 74), miR-K12-6 (SEQ ID NO 75), miR-K12-7 (SEQ ID NO 76), miR-K12-8 (SEQ ID NO 77), miR-K12-9 (SEQ ID NO 78), miR-K12-10 (SEQ ID NO 79), miR-K12-11 (SEQ ID NO 80), miR-K12-12 (SEQ ID NO 81).

FIG. 2 is an example of an individual's herpesvirus secretome data obtained from annual blood samples. In this example, the individual tested positive for HCMV vIL-10, VZV glycoprotein C, and EBV EBER1 at age of 50. The average yearly increase for vIL-10, glycoprotein C, and EBER1 in the first 10 years is 11%. The increase indicates that the individual's immune system is not controlling the herpesviruses and viral load is increasing with intermittent reactivations. Annual mini-mental state exam (MMSE) scores are included in the graph to demonstrate the presumed association of secretome levels and cognitive impairment. This individual may notice cognitive decline for the first time at age 58 which may appear soon after an illness or stressful life event. HCMV, VZV, and EBV are the most common herpesvirus types and therefore they are likely to be involved in the most common neurodegeneration, AD.

Neurodegenerations like AD is associated with blood biomarkers (Thambisetty et al. 2011). Increased amyloidogenesis in microvessel walls increases inflammatory cytokines, which leads to prothrombotic protein expression, such as increased platelet tissue factor and decreased thrombomodulin. Aβ proximately 80% of AD patients have some degree of cerebral amyloid angiography characterized by Aβ deposits in small arterials. Cerebral amyloid angiography narrows the vascular lumen and is associated with hemorrhagic bleeds, microinfarcts, and white matter pathology. Accordingly, Aβ aggregates are also found among degenerating microvessels. Vascular inflammation can activate coagulation cascade pathways resulting in vessel occlusion. At the same time, angiogenesis is upregulated in the hippocampus and proangiogenic factors are increased. All AD brains show some degree of vascular pathology and this can be detected by measuring blood coagulation biomarkers (Farkas et al. 2000; Desai et al. 2009). Measuring blood coagulation biomarkers, along with herpesvirus secreted factors and AD biomarkers, can aid in staging neurodegeneration and demonstrate causality (FIG. 3).

Accordingly, activated factor VII (SEQ ID NO 93) and von Willebrand factor (SEQ ID NO 87) are significantly higher in AD patients compared to age-matched controls (Mari et al. 1996). In some embodiments, staging and progression rate of herpesvirus-mediated neuropathology is obtained by combining the herpesvirus secretome data with a blood coagulation factor measurement, the factors selected from γ-chain-fibrinogen (SEQ ID NO 82), thrombin (SEQ ID NO 83), tissue thromboplastin (SEQ ID NO 84), plasminogen activator inhibitor type I (SEQ ID NO 85), D-dimer (SEQ ID NO 86), Von Willebrand factor (SEQ ID NO 87), Bradykinin (SEQ ID NO 88), antithrombin (SEQ ID NO 89), activated factor XII (SEQ ID NO 90), activated factor VIII (SEQ ID NO 91), Kallikrein-8 (SEQ ID NO 92), activated factor VII (SEQ ID NO 93), and activated factor X (SEQ ID NO 94). Many of these biomarkers are interchangeable for measuring coagulation activity.

Cell adhesion molecules shed from the cell surface after inflammation-induced endothelial cell activation and are released into circulation. Vascular cell adhesion molecule-1 (VCAM-1), intercellular adhesion molecule-1 (ICAM-1), and E-selectin are significantly elevated in plasma from AD patients compared to controls. However, only VCAM-1 is significantly associated with the degree of white matter lesions and short-term memory (Huang et al. 2015). In some embodiments, the staging and progression rate of herpesvirus-mediated neuropathology is obtained by combining the herpesvirus secretome data with a measure of cell adhesion molecules, the molecules selected from VCAM-1 (SEQ ID NO 95), ICAM-1 (SEQ ID NO 96), and E-selectin (SEQ ID NO 97).

Noradrenaline, also known as norepinephrine, is increased in plasma from AD patients compared to cognitively healthy age-matched controls (Pillet et al. 2020). Normal cell surface density of α₂-adrenergic receptors depends on Sorla and APP. Sorla associates with APP at its C-terminal end and restricts APP transport to the Golgi or cell membrane. Without Sorla, APP is endocytosed and merges with an early endosome where it is cleaved by β- and γ-secretase to produce Aβ. Norepinephrine-mediated activation of α₂-adrenergic receptors changes APP localization by disrupting Sorla and APP association. APP associates with activated α2-adrenergic receptors, stabilizing the α2-adrenergic receptor at the cell surface, thereby preventing arrestin 3 internalization and inhibitory feedback desensitization. The internalization of presynaptic inhibitory α2-adrenergic receptors desensitizes feedback control, increasing norepinephrine release. Accordingly, cell surface α₂-adrenergic receptor density is significantly decreased in AD brain compared to age-matched control.

One-way adrenergic neurons modulate activity is by regulating vascular tone. Norepinephrine binds α-adrenergic receptors on smooth muscle cells and pericytes to induce vasoconstriction and binds β-adrenergic receptors to induce vasorelaxation. The internalization of norepinephrine-mediated postsynaptic α₂-adrenergic receptor signaling in pericytes could result in chronic vasodilation, which would explain the depressed baroreflex in AD patients. Persistent norepinephrine-mediated postsynaptic α1-adrenergic receptor signaling can cause smooth muscle cell chronic vasoconstriction.

Aβ deposits are a defining pathological feature in AD brains. Norepinephrine activated β₂-adrenergic receptor induces cAMP-mediated APP gene expression and amyloidogenic processing in astrocytes. Increased norepinephrine mediated α₁-adrenergic receptor signaling stimulates glutamate and ATP release from astrocytes. Activation of α₂-adrenergic receptors upregulates calcium oscillations in astrocytes, which increases the release of the inhibitory neurotransmitter GABA. GABA in turn reduces calcium oscillation in neurons. However, APP deficiency would decrease α₂-adrenergic receptor stabilization at the astrocyte cell-surface resulting in reduced norepinephrine-mediated GABA release. Accordingly, mice expressing a homozygous loss-of-function APP mutation show electrophysiology consistent with GABAergic deficit. GABA is also involved in regulating adult neurogenesis and neuritogenesis. Thus, loss of tonic GABA release from astrocytes would also explain reduced neurogenesis in the AD dentate gyrus.

Neurons and astrocytes express the ionotropic NMDA receptor (NMDAR). Aβ can bind to NMDARs and activate calcium influx. In neurons, sustained intracellular calcium can induce long term depression, leading to neurite loss. A higher intracellular calcium concentration increases intracellular chloride. Increased intracellular chloride causes chloride efflux in GABA-activated chloride channels, resulting in depolarization. This situation resembles development, wherein GABA from inhibitory interneurons acts as an excitatory neurotransmitter, stimulating neurite outgrowth. This could explain the observance of abnormal neurite outgrowth surrounding Aβ deposits in AD brain. The activation of neuronal α₁- and α₂-adenergic receptors reduces the NMDAR-mediated excitatory post-synaptic potential (EPSP) amplitude but not the paired pulse response. In neurons, Aβ oligomers may bind allosterically to α₂-adenergic receptors to enhance norepinephrine-mediated G-protein activation. Decreased expression of regulator of G-protein signaling 2 (RGS2) in AD brain would potentiate decreased NMDAR EPSP. Thus, increased norepinephrine and Aβ-mediated α1- and α2-adrenergic receptor activation could reduce neuronal EPSP amplitudes, which could affect synaptic maintenance.

Norepinephrine is a chemokine for macrophages and switches cells to the anti-inflammatory M2 phenotype. Norepinephrine can also suppress NK cell activity and dendritic cell maturation. Moreover, norepinephrine activates corticotropin-releasing hormone cells to increase HPA mediated cortisol release. Thus, herpesviruses make the host more hospitable by upregulating norepinephrine. In summary, deficient APP-mediated α₂-adrenergic receptor stabilization increases norepinephrine (noradrenaline) to cause, (1) reduced astrocytic GABA, (2) reduced neurogenesis, (3) reduced EPSP amplitudes, and (4) immunosuppression.

Hyperactive alphaherpesvirus infected LC adrenergic neurons could also increase norepinephrine. Notably, mouse autonomic neurons infected with an alphaherpesvirus and viewed with live calcium imaging show aberrant synchronous firing. Locus coeruleus adrenergic neurons project axons throughout the brain to control optimal circadian homeostatic activity through the release of norepinephrine and coordinated peptides. Herpesviruses evolved to entrain viral activity with their host circadian rhythm. For instance, the HSV ICPO transactivator has binding affinity to cellular BMAL1. In the morning, when BMAL1 level is high, BMAL1 binds the viral ICPO transactivator to block herpesvirus activity. As BMAL1 falls through the day, ICPO becomes free to activate viral transcription. Nocturnal HSV-mediated norepinephrine release could explain why AD patients experience sleep disorders.

Combining herpesvirus secretome levels with coagulation factors, cell adhesion molecules, noradrenaline and other AD-related biomarkers can aid in diagnosis and predict individuals who will progress from mild cognitive impairment to AD-type dementia. For example, Amyloid-β₁₋₄₂ is significantly reduced in plasma from AD patients compared to cognitively healthy age-matched controls (Nakamura et al. 2018) Thus, in some embodiments, herpesvirus secretome factors, coagulation factors, cell adhesion molecules, noradrenaline and additional biomarkers, selected from amyloid-β_{β1-42} (SEQ ID NO 98), alpha-1-microglobulin (SEQ ID NO 99), complement factor I (SEQ ID NO 100), complement 3a (SEQ ID NO 101), are combined to provide accurate staging and progression rate of herpesvirus-mediated neuropathology. In some patients, herpesvirus secretome concentrations will increase with age, as intermittent reactivations increase the viral load.

FIG. 3 shows an example of using AD biomarkers along with the herpesvirus secretome to aid in diagnosis and provide neurodegeneration staging and progression rate in a hypothetical individual. The graph exhibits annual herpesvirus secretome level along with biomarkers, γ-chain-fibrinogen, and noradrenaline. The biomarkers divide pathology into 3 stages. In stage 1, the expression of both biomarkers is negligible indicating an absence of pathology. In stage 2, noradrenaline is increased along with subacute chronic VZV reactivation as shown by increasing VZV gC levels. In stage 3, γ-chain-fibrinogen is increased from vascular inflammation, causing microvessel coagulation and ischemia. The VZV gC level increased an average of 14% per year between age 50-60, increasing approximately 400% in 10 years. During this time, cognitive decline may be undetected due to the gradualness and cognitive reserve. In FIG. 4, the hypothetical individual tests positive for HCMV vIL-10 and VZV gC but negative for EBV EBER1. Secretome levels and AD biomarkers are stable from age 50-69, indicating that the individual is controlling herpesvirus activity and pathology is at stage 0. However, VZV could still reactivate in this hypothetical individual at age 80, and by age 84, after a significant number of VZV infected LC neurons have died, the individual begins a steep cognitive decline. The inflection point could coincide with immunosuppression or a traumatic life event. The ability to predict herpesvirus-mediated neurodegeneration based on herpesvirus secretome levels is based on established nominal levels and an individual's baseline.

In some embodiments, herpesvirus secretome proteins are detected with an Enzyme-Linked ImmunoSorbent Assay (ELISA). A rapid ELISA can be performed with just 0.05 ml of whole blood at room temperature with optical density results in 20 minutes (Hirose et al. 2005). Reagent cost and volumes are minimal. Kits containing preabsorbed antibodies targeting secreted herpesvirus factors and reagents are vastly more precise and economical than the current AD diagnostics. Most antibodies for secretome and biomarker proteins are available from antibody manufacturers. The antibodies or aptamers requiring custom production can be obtained by custom antibody production services. In some cases, an amplification system is used to increase the sensitivity of both the ELISA. For example, ThermoFisher's ELF 97 phosphate substrate produces a precipitate that upon excitation emits a robust fluorescent signal. In some cases, a nucleic acid amplification system for ELISA is used to increase protein detection sensitivity in the femtomolar r

In some embodiments, the herpesvirus secreted factors are identified and quantified using mass spectrometry (Zhang et al. 2010). Proteins secreted from select herpesvirus infected cells are reduced, trypsin digested, and isoelectric-fractionated. The cleaned peptides are analyzed by integrated liquid chromatography-Easy-nLC 1000 (Proxeon, Fischer Scientific) and mass spectrometry Q-Exactive (Thermo Fischer Scientific). The mass spectrometry data is analyzed with MaxQuant to identify the optimal and unique peptides. Parameters for a targeted multiplex mass spectrometry assay are optimized using parallel reaction monitoring (Wee et al. 2019). In summary, an extremely sensitive one-shot MS assay can identify and quantify the presence of multiple secreted herpesvirus factors in an individual's blood sample.

In some embodiments, herpesvirus proteins and RNA are isolated from exosomes. Exosomes are isolated from blood serum. After venipuncture, blood is allowed blood to clot in the tube for one hour and then centrifuged for 15 minutes at 2500 rpm to obtain serum. Alternately, exosomes are obtained from plasma after centrifuging whole blood in an anticoagulant tube. The serum or plasma is transferred to a vial for exosome isolation. Exosomes are isolated by adding ThermoFisher Scientific's exosome isolation reagent (Catalog no. 4478360) or a solution of less than 0.1% sodium azide to serum and incubating for 30 min. at 2° C. to 8° C. Precipitated exosomes are centrifuged at 10,000×g for 10 min and the pellet is resuspended in phosphate buffer saline. The pelleted exosomes can be stored at −20° C. The RNA and protein are released from the exosome by organic extraction and immobilization on glass-fiber filters.

In some cases, exosome isolated RNAs are quantified by reverse transcription to cDNA and quantitative real-time PCR amplification using published methods (Aromseree et al. 2017). Primer sequences for amplifying all non-coding RNAs disclosed herein are published. In some cases, RNA levels are measured by custom microarray builds. In some cases, RNA levels are measured using a multiplex label-free optical biosensor assay, wherein the RNA hybridizes to a DNA probe that is then detected by an antibody. The antibody binding, and especially the addition of a secondary antibody, alters the surface density (Zanchetta et al. 2020; Salina et al. 2015). Similarly, RNA levels are measured with picomolar sensitivity by a modified RNA ELISA assay, such as the Quantikine by R&D Systems. The extracted RNA is detected by hybridization to a complementary oligonucleotide probe, or aptamer, labeled with biotin and digoxigenin and transferred to a streptavidin-coated microplate where the RNA/probe hybrid is captured. The unbound probe is removed by rinsing and anti-digoxigenin alkaline phosphatase conjugate is added. Unbound conjugate is removed, and a substrate solution is added. Color develops in proportion to the amount of target RNA in the sample. The intensity of the color, or optical density, is measured with a spectrophotometer plate reader.

Herpesvirus factor concentrations are determined by interpolating values from a standard curve derived from the purified recombinant protein of known concentrations. Reference molecules or total protein are measured to normalize repeated measures, negating sample viscosity variability. A herpesvirus secretome screening serves to identify the herpesvirus types infecting an individual. Subsequently, secretome factors are quantified and compared against a reference point or personal baseline. All future measurements are referenced to the baseline measurement to determine the progression rate. Collective patient data will define secretome reference protein levels associated with symptom onset and establish thresholds for predicting symptom onset. An algorithm incorporating secretome variables is used to project progression risk. The algorithm uses epidemiology data for predicting progression rate or the date of symptom onset. Variables included in the algorithm are secretome levels, neurodegeneration biomarkers, the herpesvirus types and strains, age, sex, diet, education level, sleep efficiency, comorbidities, exercise, occupation, and stress level. Regression weights are obtained from multiple regression of epidemiological data. Measuring herpesvirus secreted protein and RNA levels can provide rapid feedback on treatment efficacy in AD clinical trials, including trials for therapeutic herpesvirus vaccines.

In some embodiments, reagents to screen select secretome factors in blood samples are assembled into a diagnostic ELISA kit, wherein the kit comprises: (a) an antibody or aptamer which specifically binds to a herpesvirus secretome protein or RNA, (b) a solid matrix or microplate well to which a capture antibody or aptamer is bound, (c) a secondary antibody or aptamer permitting the selective formation of a complex of a sample antigen between two layers of antibodies or aptamers, and (d) reagents for detection. In some cases, a screening kit includes an assortment of antibodies or aptamers that target secretome proteins or RNA from the eight herpesviruses as listed above. In some cases, the kit is customized to include antibodies or aptamers that target secreted factors from specific herpesvirus types, along with the recombinant proteins standards for measuring concentration. In some cases, the kit includes an antibody or aptamer that targets secreted herpesvirus factors and a coagulation factor, cellular adhesion molecule, and/or Alzheimer's disease biomarker.

In one example, an annual screening ELISA kit would contain preabsorbed antibodies to HCMV pp150 (SEQ ID NO 4) and vIL-10 (SEQ ID NO 10); HSV-1 VP22 (SEQ ID NO 54); HSV-2 glycoprotein G (SEQ ID NO 59); HHV-6A glycoprotein B (SEQ ID NO 21) and IE2 (SEQ ID NO 19); HHV-6B U83 (SEQ ID NO 23); HHV-7 glycoprotein B (SEQ ID NO 28) and IE2 (SEQ ID NO 26); EBV gp350 (SEQ ID NO 17), and ebvIL-10 (SEQ ID NO 53). In some embodiments, screening results are used to select a herpesvirus type-specific quantitative kit. The quantitative kit contains reagents for detecting the concentration of secretome factors, reference targets, and concentration standards. This type of kit might be used in a clinical trial testing herpesvirus treatment efficacy. In some embodiments, the kit measures coagulation factors, cell adhesion molecules, and AD biomarkers along with herpesvirus secretome to aid in diagnosis and predict neurodegeneration stage and progression rate.

In some embodiments, blood from an individual is blotted on paper, dried, and shipped from healthcare providers to a processing center (Björkesten et al. 2017).

### CONCLUSION

The described methods and embodiments can precisely, and at low cost, identify the active herpesvirus and stage herpesvirus-mediated neurodegeneration. The method of detecting secreted herpesvirus factors will finally establish that herpesvirus types and strains and combinations thereof contribute to a variety of neurodegenerations. Furthermore, it will usher in a new era of therapeutic discovery to prevent herpesvirus-mediated neurodegeneration.

### BRIEF DESCRIPTION OF THE DRAWINGS

FIG. 1 shows the wide range of HCMV vIL-10 protein levels among individuals. Shown with permission from the author and copyright owner Vivian Young (Vivian P. Young, Master's Thesis: Detection of HCMV viral IL-10 (vIL-10) in healthy blood donors, The University of San Francisco, published May 17, 2015).

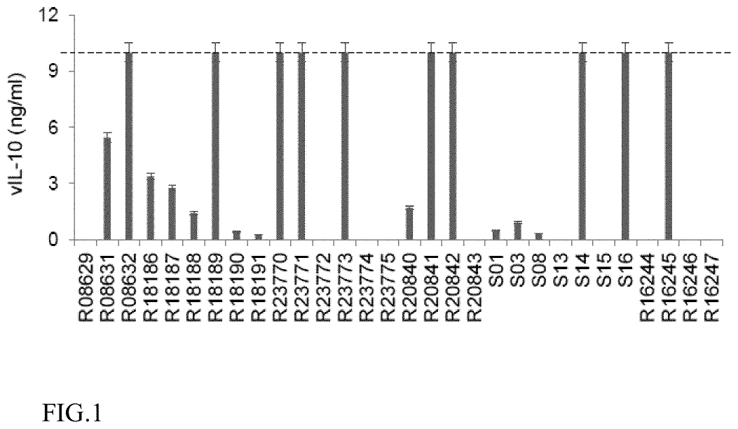

FIG. 2 depicts the annual levels of HCMV, VZV, and EBV secreted proteins in blood samples from a hypothetical individual along with annual Mini-Mental State Exam scores.

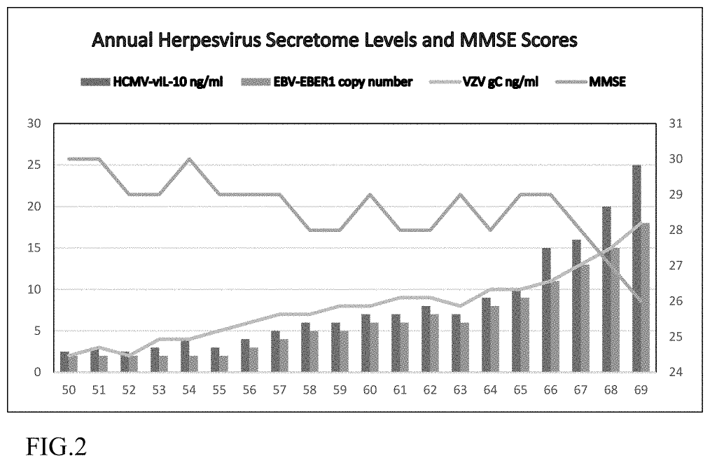

FIG. 3 depicts the annual herpesvirus levels of a hypothetical individual along with AD biomarkers, γ-fibrinogen, and noradrenaline, wherein the biomarkers levels aid in disease staging and progression.

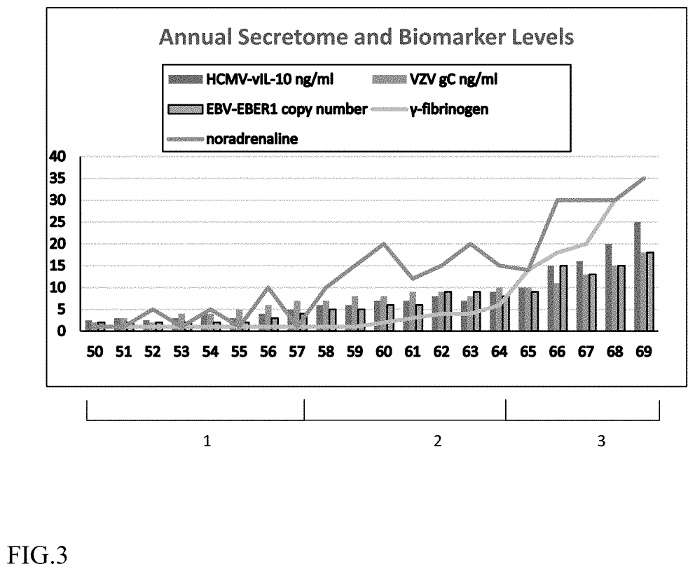

FIG. 4 depicts the annual herpesvirus levels of a hypothetical individual along with AD biomarkers, γ-fibrinogen, and noradrenaline, wherein the individual is positive for HCMV and VZV, but shows no disease progression, stage 0.

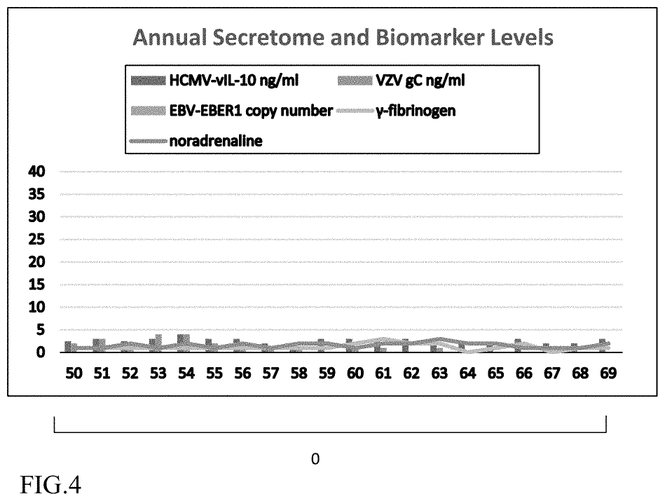

### SEQUENCE DISCLOSURE

The sequences are disclosed in a 339 kB text file named, “10005B—US—NP_Sequences-as-filed”, created on 2021/01/10.

### Definitions

As used herein, the term “AD patient”, and refer to an individual who has been diagnosed with Alzheimer's Disease or has been given a probable diagnosis of Alzheimer's Disease.

As used herein, the phrase “AD biomarker” refers to a biomarker that is associated with an AD diagnosis.

The term “aptamer”, as used herein, refers to an oligonucleotide or peptide molecule that binds to a specific target molecule.

The term norepinephrine and noradrenaline refer to the same chemical.

The term RNA herein refers to non-coding RNA and includes long non-coding RNA and miRNA.

The term “secreted factor” or “secreted protein” includes herpesvirus RNA or proteins secreted in capsid types A and B, dense bodies, RNA and proteins secreted in a complex with other proteins, and RNA and proteins secreted in exosomes.

As used herein, methods for “aiding diagnosis” refer to methods that assist in making a clinical determination regarding a contributing cause of AD or mild cognitive impairment. Accordingly, for example, a method of aiding diagnosis of AD can comprise measuring the amount of one or more AD biomarkers in a blood sample from an individual.

As used herein, the term “staging” refers to sorting individuals into different classes or strata based on the level of herpesvirus secreted proteins and neurodegeneration-related biomarkers. Staging individuals based on blood biomarkers can correlate with cognitive impairment and aids in disease staging (e.g., mild, moderate, advanced, etc.).

As used herein, the term “predicting” means determining the probability that an individual will develop symptoms of neurodegeneration.

As used herein, the phrase “neurodegeneration” refers to a disease or disorder of the central nervous system. Neurodegeneration includes multiple sclerosis, neuropathies, AD, Parkinson's disease, amyotrophic lateral sclerosis (ALS), mild cognitive impairment (MCI), retinitis, herpes zoster ophthalmicus, and frontotemporal dementia.

As used herein, the phrase “neuropathology” refers to the presence of physical or physiological abnormality.

A “blood sample” is a biological sample that is derived from peripheral blood. A blood sample may be, for example, whole blood, plasma, or serum. The definition also includes samples that have been manipulated in any way after their procurement, such as by treatment with reagents, solubilization, or enrichment for certain components, such as proteins or polynucleotides.

An “individual” is a mammal, more preferably a human. Mammals include, but are not limited to, humans, primates, farm animals, sport animals, rodents, and pets.

### REFERENCES

Allnutt, Mary Alice; Johnson, Kory; Bennett, David A.; Connor, Sarah M.; Troncoso, Juan C.; Pletnikova, Olga et al. (2019): Human Herpesvirus-6 (HHV-6) Detection in Alzheimer's Disease Cases and Controls Across Multiple Cohorts. In SSRN Journal. DOI: 10.2139/ssrn.3464033.
André Parent (1996): Carpenter's Human Neuroanatomy. Ninth. Baltimore: Williams and Wilkins.
Andrés-Benito et. al. (2017): Locus coeruleus at asymptomatic early and middle Braak stages of neurofibrillary tangle pathology.
Aoki, Y.; Yarchoan, R.; Wyvill, K.; Okamoto, S.; Little, R. F.; Tosato, G. (2001): Detection of viral interleukin-6 in Kaposi sarcoma-associated herpesvirus-linked disorders. In Blood 97 (7), pp. 2173-2176. DOI: 10.1182/blood.V97.7.2173.
Aromseree, Sirinart; Middeldorp, Jaap M.; Pientong, Chamsai; van Eijndhoven, Monique; Ramayanti, Octavia; Lougheed, Sinead M. et al. (2017): High Levels of EBV-Encoded RNA 1 (EBER1) Trigger Interferon and Inflammation-Related Genes in Keratinocytes Expressing HPV16 E6/E7. In PloS one 12 (1), e0169290. DOI: 10.1371/journal.pone.0169290.
Arvin A, Campadelli-Fiume G, Mocarski E, et al (Ed.) (2007): Human Herpesviruses: Biology, Therapy, and Immunoprophylaxis. Cambridge: University Press.
Baillargeon, Jacques; Leach, Charles T.; Deng, Jian-Hong; Gao, Shou-Jiang; Jenson, Hal B. (2002): High prevalence of human herpesvirus 8 (HHV-8) infection in south Texas children. In Journal of medical virology 67 (4), pp. 542-548. DOI: 10.1002/jmv.10136.
Bakker, Paul I. W. de; McVean, Gil; Sabeti, Pardis C.; Miretti, Marcos M.; Green, Todd; Marchini, Jonathan et al. (2006): A high-resolution HLA and SNP haplotype map for disease association studies in the extended human MHC. In Nature genetics 38 (10), pp. 1166-1172. DOI: 10.1038/ng1885.
Baringer, J. R.; Pisani, P. (1994): Herpes simplex virus genomes in human nervous system tissue analyzed by polymerase chain reaction. In Annals of neurology 36 (6), pp. 823-829. DOI: 10.1002/ana.410360605.
Barnes, Lisa L.; Capuano, Ana W.; Aiello, Alison E.; Turner, Arlener D.; Yolken, Robert H.; Torrey, E. Fuller; Bennett, David A. (2015): Cytomegalovirus infection and risk of Alzheimer disease in older black and white individuals. In The Journal of infectious diseases 211 (2), pp. 230-237. DOI: 10.1093/infdis/jiu437.
Bigger, J. E.; Tanigawa, M.; Zhang, M.; Atherton, S. S. (2000): Murine cytomegalovirus infection causes apoptosis of uninfected retinal cells. In Investigative ophthalmology & visual science 41 (8), pp. 2248-2254.
Björkesten, Johan; Enroth, Stefan; Shen, Qiujin; Wik, Lotta; Hougaard, David M.; Cohen, Arieh S. et al. (2017): Stability of Proteins in Dried Blood Spot Biobanks. In Molecular & cellular proteomics: MCP 16 (7), pp. 1286-1296. DOI: 10.1074/mcp.RA117.000015.
Casiraghi, Costanza; Dorovini-Zis, Katerina; Horwitz, Marc S. (2011): Epstein-Barr virus infection of human brain microvessel endothelial cells. A novel role in multiple sclerosis. In Journal of neuroimmunology 230 (1-2), pp. 173-177. DOI: 10.1016/j.jneuroim.2010.08.003.
Cesarman, Ethel; Damania, Blossom; Krown, Susan E.; Martin, Jeffrey; Bower, Mark; Whitby, Denise (2019): Kaposi sarcoma. In Nature reviews. Disease primers 5 (1), p. 9. DOI: 10.1038/s41572-019-0060-9.
Chandran, B.; Smith, M. S.; Koelle, D. M.; Corey, L.; Horvat, R.; Goldstein, E. (1998): Reactivities of human sera with human herpesvirus-8-infected BCBL-1 cells and identification of HHV-8-specific proteins and glycoproteins and the encoding cDNAs. In Virology 243 (1), pp. 208-217. DOI: 10.1006/viro.1998.9055.
Chen, Huang-Hsi; Perng, Wuu-Tsun; Chiou, Jeng-Yuan; Wang, Yu-Hsun; Huang, Jing-Yang; Wei, James Cheng-Chung (2019): Risk of dementia among patients with Sjogren's syndrome. A nationwide population-based cohort study in Taiwan. In Seminars in arthritis and rheumatism 48 (5), pp. 895-899. DOI: 10.1016/j.semarthrit.2018.06.007.
Chen, Lei; Lagunoff, Michael (2007): The KSHV viral interleukin-6 is not essential for latency or lytic replication in BJAB cells. In Virology 359 (2), pp. 425-435. DOI: 10.1016/j.virol.2006.09.044.
Chiara, Giovanna de; Marcocci, Maria Elena; Civitelli, Livia; Argnani, Rafaela; Piacentini, Roberto; Ripoli, Cristian et al. (2010): APP processing induced by herpes simplex virus type 1 (HSV-1) yields several APP fragments in human and rat neuronal cells. In PloS one 5 (11), e13989. DOI: 10.1371/journal.pone.0013989.
Chung, Sun Ju; Kim, Mi-Jung; Kim, Young Jin; Kim, Juyeon; You, Sooyeoun; Jang, Eun Hye et al. (2014): CR1, ABCA7, and APOE genes affect the features of cognitive impairment in Alzheimer's disease. In Journal of the neurological sciences 339 (1-2), pp. 91-96. DOI: 10.1016/j.jns.2014.01.029.
Ciaramella, Antonio; Salani, Francesca; Bizzoni, Federica; Orfei, Maria Donata; Caltagirone, Carlo; Spalletta, Gianfranco; Bossu, Paola (2016): Myeloid dendritic cells are decreased in peripheral blood of Alzheimer's disease patients in association with disease progression and severity of depressive symptoms. In J Neuroinflammation 13 (1), p. 383. DOI: 10.1186/s12974-016-0483-0.
Collard, C. D.; Bukusoglu, C.; Agah, A.; Colgan, S. P.; Reenstra, W. R.; Morgan, B. P.; Stahl, G. L. (1999): Hypoxia-induced expression of complement receptor type 1 (CR1, CD35) in human vascular endothelial cells. In The American journal of physiology 276 (2), C450-8. DOI: 10.1152/ajpcell.1999.276.2.C450.
Cone, R. W.; Huang, M. L.; Ashley, R.; Corey, L. (1993): Human herpesvirus 6 DNA in peripheral blood cells and saliva from immunocompetent individuals. In Journal of Clinical Microbiology 31 (5), pp. 1262-1267.
Deatly, A. M.; Haase, A. T.; Fewster, P. H.; Lewis, E.; Ball, M. J. (1990): Human herpes virus infections and Alzheimer's disease. In Neuropathology and applied neurobiology 16 (3), pp. 213-223.
Desai, Brinda S.; Schneider, Julie A.; Li, Jia-Liang; Carvey, Paul M.; Hendey, Bill (2009): Evidence of angiogenic vessels in Alzheimer's disease. In Journal of neural transmission (Vienna, Austria: 1996) 116 (5), pp. 587-597. DOI: 10.1007/s00702-009-0226-9.
Di Carlo, Paola; Trizzino, Marcello; Titone, Lucina; Capra, Giuseppina; Colletti, Piero; Mazzola, Giovanni et al. (2011): Unusual MRI findings in an immunocompetent patient with EBV encephalitis. A case report. In BMC Med Imaging 11 (1), p. 481. DOI: 10.1186/1471-2342-11-6.
Dumortier, Jerome; Streblow, Daniel N.; Moses, Ashlee V.; Jacobs, Jon M.; Kreklywich, Craig N.; Camp, David et al. (2008): Human Cytomegalovirus Secretome Contains Factors That Induce Angiogenesis and Wound Healing. In JVI 82 (13), pp. 6524-6535. DOI: 10.1128/JVI.00502-08.
Ehlenbach, William J.; Hough, Catherine L.; Crane, Paul K.; Haneuse, Sebastien J. P. A.; Carson, Shannon S.; Curtis, J. Randall; Larson, Eric B. (2010): Association between acute care and critical illness hospitalization and cognitive function in older adults. In JAMA 303 (8), pp. 763-770. DOI: 10.1001/jama.2010.167.
Elliott, Gillian; O'Hare, Peter (1997): Intercellular Trafficking and Protein Delivery by a Herpesvirus Structural Protein. In Cell 88 (2), pp. 223-233. DOI: 10.1016/s0092-8674(00)81843-7.
England, Bryant R.; Mikuls, Ted R.; Xie, Fenglong; Yang, Shuo; Chen, Lang; Curtis, Jeffrey R. (2017): Herpes Zoster as a Risk Factor for Incident Giant Cell Arteritis. In Arthritis & rheumatology (Hoboken, N.J.). DOI: 10.1002/art.40236.
Farkas, E.; Jong, G. I. de; Vos, R. A. de; Jansen Steur, E. N.; Luiten, P. G. (2000): Pathological features of cerebral cortical capillaries are doubled in Alzheimer's disease and Parkinson's disease. In Acta Neuropathol 100 (4), pp. 395-402. DOI: 10.1007/s004010000195.
Fonseca, Maria I.; Chu, Shuhui; Pierce, Aimee L.; Brubaker, William D.; Hauhart, Richard E.; Mastroeni, Diego et al. (2016): Analysis of the Putative Role of CR1 in Alzheimer's Disease. Genetic Association, Expression and Function. In PloS one 11 (2), e0149792. DOI: 10.1371/journal.pone.0149792.
Fraser, N. W.; Lawrence, W. C.; Wroblewska, Z.; Gilden, D. H.; Koprowski, H. (1981): Herpes simplex type 1 DNA in human brain tissue. In Proceedings of the National Academy of Sciences 78 (10), pp. 6461-6465. DOI: 10.1073/pnas.78.10.6461.
Gate, David; Saligrama, Naresha; Leventhal, Olivia; Yang, Andrew C.; Unger, Michael S.; Middeldorp, Jinte et al. (2020): Clonally expanded CD8 T cells patrol the cerebrospinal fluid in Alzheimer's disease. In Nature 577 (7790), pp. 399-404. DOI: 10.1038/s41586-019-1895-7.
Geerling, Joel C.; Shin, Jung-Won; Chimenti, Peter C.; Loewy, Arthur D. (2010): Paraventricular hypothalamic nucleus: axonal projections to the brainstem. In THE JOURNAL OF COMPARATIVE NEUROLOGY 518 (9), pp. 1460-1499. DOI: 10.1002/cne.22283.
Granstedt, Andrea E.; Bosse, Jens B.; Thiberge, Stephan Y.; Enquist, Lynn W. (2013): In vivo imaging of alphaherpesvirus infection reveals synchronized activity dependent on axonal sorting of viral proteins. In Proceedings of the National Academy of Sciences of the United States of America 110 (37), E3516-25. DOI: 10.1073/pnas.1311062110.
Hemling, Niina; Roytta, Matias; Rinne, Juha; Pollanen, Paju; Broberg, Eeva; Tapio, Virpi et al. (2003): Herpesviruses in brains in Alzheimer's and Parkinson's diseases. In Ann Neurol. 54 (2), pp. 267-271. DOI: 10.1002/ana.10662.
Hirose, Yoshinori; Kirinoki, Masashi; Lipayon, Ignacio L.; Blas, Bayani L.; Chigusa, Yuichi; Matsuda, Hajime (2005): Development of the rapid and simplified ELISA (WHOLE BLOOD-ELISA) using samples of Schistosoma japonicum-infected human whole blood. In The Southeast Asian journal of tropical medicine and public health 36 (1), pp. 72-78.
Hou, Tsung-Yun; Hsu, Hui-Ching; Lin, Tzu-Min; Chang, Yu-Sheng; Chen, Wei-Sheng; Kuo, Pei-I et al. (2019): Higher risk of dementia in primary Sjogren's syndrome. In Annals of clinical and translational neurology 6 (4), pp. 633-641. DOI: 10.1002/acn3.737.
Hu, Benxia; Li, Xin; Huo, Yongxia; Yu, Yafen; Zhang, Qiuping; Chen, Guijun et al. (2016): Cellular responses to HSV-1 infection are linked to specific types of alterations in the host transcriptome. In Scientific reports 6, p. 28075. DOI: 10.1038/srep28075.
Huang, Chi-Wei; Tsai, Meng-Han; Chen, Nai-Ching; Chen, Wei-Hsi; Lu, Yan-Ting; Lui, Chun-Chung et al. (2015): Clinical significance of circulating vascular cell adhesion molecule-1 to white matter disintegrity in Alzheimer's dementia. In Thrombosis and haemostasis 114 (6), pp. 1230-1240. DOI: 10.1 160/TH14-11-0938.
Iwakiri, Dai; Zhou, Li; Samanta, Mrinal; Matsumoto, Misako; Ebihara, Takashi; Seya, Tsukasa et al. (2009): Epstein-Barr virus (EBV)-encoded small RNA is released from EBV-infected cells and activates signaling from Toll-like receptor 3. In The Journal of experimental medicine 206 (10), pp. 2091-2099. DOI: 10.1084/jem.20081761.
Jabs, W. J.; Paulsen, M.; Wagner, H. J.; Kirchner, H.; Kltter, H. (1999): Analysis of Epstein-Barr virus (EBV) receptor CD21 on peripheral B lymphocytes of long-term EBV-adults. In Clinical & Experimental Immunology 116 (3), pp. 468-473. DOI: 10.1046/j.1365-2249.1999.00912.x.
James, Lisa M.; Christova, Peka; Lewis, Scott M.; Engdahl, Brian E.; Georgopoulos, Angeliki; Georgopoulos, Aβ ostolos P. (2018): Protective Effect of Human Leukocyte Antigen (HLA) Allele DRB1*13:02 on Age-Related Brain Gray Matter Volume Reduction in Healthy Women. In EBioMedicine 29, pp. 31-37. DOI: 10.1016/j.ebiom.2018.02.005.
Jeong, Hyun-Hwan; Liu, Zhandong (2019): Are HHV-6A and HHV-7 Really More Abundant in Alzheimer's Disease? In Neuron 104 (6), pp. 1034-1035. DOI: 10.1016/j.neuron.2019.11.009.
Jha, Hem Chandra; Mehta, Devan; Lu, Jie; El-Naccache, Darine; Shukla, Sanket K.; Kovacsics, Colleen et al. (2015): Gammaherpesvirus Infection of Human Neuronal Cells. In mBio 6 (6), e01844-15. DOI: 10.1128/mBio.01844-15.
Jones, K.; Rivera, C.; Sgadari, C.; Franklin, J.; Max, E. E.; Bhatia, K.; Tosato, G. (1995): Infection of human endothelial cells with Epstein-Barr virus. In The Journal of experimental medicine 182 (5), pp. 1213-1221. DOI: 10.1084/jem.182.5.1213.
Karkhaneh, Reza; Lashay, Alireza; Ahmadraji, Aliasghar (2016): Cytomegalovirus retinitis in an immunocompetent patient. A case report. In Journal of current ophthalmology 28 (2), pp. 93-95. DOI: 10.1016/j.joco.2015.12.003.
Kittur, S. D.; Hoh, J. H.; Kawas, C. H.; Hayward, G. S.; Endo, H.; Adler, W. H. (1992): A molecular hybridization study for the presence of Herpes simplex, cytomegalovirus and Epstein-Barr virus in brain and blood of Alzheimer's disease patients. In Archives of Gerontology and Geriatrics 15 (1), pp. 35-41. DOI: 10.1016/0167-4943(92)90038-6.
Koronyo, Yosef, Biggs, David; Barron, Ernesto; Boyer, David S.; Pearlman, Joel A.; Au, William J. et al. (2017): Retinal amyloid pathology and proof-of-concept imaging trial in Alzheimer's disease. In JCI insight 2 (16). DOI: 10.1172/jci.insight.93621.
Kosenko, Elena A.; Tikhonova, Lyudmila A.; Montoliu, Carmina; Barreto, George E.; Aliev, Gjumrakch; Kaminsky, Yury G. (2017): Metabolic Abnormalities of Erythrocytes as a Risk Factor for Alzheimer's Disease. In Frontiers in neuroscience 11, p. 728. DOI: 10.3389/fnins.2017.00728.
Kosugi, I.; Shinmura, Y.; Li, R. Y.; Aiba-Masago, S.; Baba, S.; Miura, K.; Tsutsui, Y. (1998): Murine cytomegalovirus induces apoptosis in non-infected cells of the developing mouse brain and blocks apoptosis in primary neuronal culture. In Acta Neuropathol 96 (3), pp. 239-247. DOI: 10.1007/s004010050890.
Kwok, Hin; Chan, Koon Wing; Chan, Kwok Hung; Chiang, Alan Kwok Shing (2015): Distribution, persistence and interchange of Epstein-Barr virus strains among PBMC, plasma and saliva of primary infection subjects. In PloS one 10 (3), e0120710. DOI: 10.1371/journal.pone.0120710.
Levesque-Sergerie, Jean-Philippe; Duquette, Mathieu; Thibault, Catherine; Delbecchi, Louis; Bissonnette, Nathalie (2007): Detection limits of several commercial reverse transcriptase enzymes. Impact on the low- and high-abundance transcript levels assessed by quantitative RT-PCR. In BMC molecular biology 8, p. 93. DOI: 10.1186/1471-2199-8-93.
Li, Huifen; Weng, Peter; Najarro, Kevin; Xue, Qian-Li; Semba, Richard D.; Margolick, Joseph B.; Leng, Sean X. (2014): Chronic CMV infection in older women. Longitudinal comparisons of CMV DNA in peripheral monocytes, anti-CMV IgG titers, serum IL-6 levels, and CMV pp65 (NLV)-specific CD8(+) T-cell frequencies with twelve year follow-up. In Experimental gerontology 54, pp. 84-89. DOI: 10.1016/j.exger.2014.01.010.
Lin, Shih-Yi; Hsu, Wu-Huei; Lin, Cheng-Chieh; Lin, Cheng-Li; Yeh, Hung-Chieh; Kao, Chia-Hung (2019): Association of Transfusion With Risks of Dementia or Alzheimer's Disease. A Population-Based Cohort Study. In Frontiers in psychiatry 10, p. 571. DOI: 10.3389/fpsyt.2019.00571.
Lin, Woan-Ru; Wozniak, Matthew A.; Wilcock, Gordon K.; Itzhaki, Ruth F. (2002): Cytomegalovirus is present in a very high proportion of brains from vascular dementia patients. In Neurobiology of Disease 9 (1), pp. 82-87. DOI: 10.1006/nbdi.2001.0465.
Lodding, Isabelle P.; Mocroft, Amanda; da Cunha Bang, Caspar; Gustafsson, Finn; Iversen, Martin; Kirkby, Nikolai et al. (2018): Impact of CMV PCR Blips in Recipients of Solid Organ and Hematopoietic Stem Cell Transplantation. In Transplantation direct 4 (6), e355. DOI: 10.1097/TXD.0000000000000787.
Lu, Rui-Chun; Yang, Wu; Tan, Lin; Sun, Fu-Rong; Tan, Meng-Shan; Zhang, Wei et al. (2017): Association of HLA-DRB1 polymorphism with Alzheimer's disease. A replication and meta-analysis. In Oncotarget 8 (54), pp. 93219-93226. DOI: 10.18632/oncotarget.21479.
Luczynski, Pauline; Laule, Cornelia; Hsiung, Ging-Yuek Robin; Moore, G. R. Wayne; Tremlett, Helen (2019): Coexistence of Multiple Sclerosis and Alzheimer's disease. A review. In Multiple sclerosis and related disorders 27, pp. 232-238. DOI: 10.1016/j.msard.2018.10.109.
Luganini, Anna; Terlizzi, Maria E.; Gribaudo, Giorgio (2016): Bioactive Molecules Released From Cells Infected with the Human Cytomegalovirus. In Frontiers in microbiology 7, p. 715. DOI: 10.3389/fmicb.2016.00715.
Lupey-Green, Lena N.; Caruso, Lisa B.; Madzo, Jozef, Martin, Kayla A.; Tan, Yinfei; Hulse, Michael; Tempera, Italo (2018): PARP1 Stabilizes CTCF Binding and Chromatin Structure To Maintain Epstein-Barr Virus Latency Type. In J Virol 92 (18). DOI: 10.1128/JVI.00755-18.
Lurain, Nell S.; Hanson, Barbara A.; Martinson, Jeffrey; Leurgans, Sue E.; Landay, Alan L.; Bennett, David A.; Schneider, Julie A. (2013): Virological and immunological characteristics of human cytomegalovirus infection associated with Alzheimer disease. In The Journal of infectious diseases 208 (4), pp. 564-572. DOI: 10.1093/infdis/jit210.
Mari, D.; Parnetti, L.; Coppola, R.; Bottasso, B.; Reboldi, G. P.; Senin, U.; Mannucci, P. M. (1996): Hemostasis abnormalities in patients with vascular dementia and Alzheimer's disease. In Thrombosis and haemostasis 75 (2), pp. 216-218.
Martin J N (2007): The epidemiology of KSHV and its association with malignant disease. Human Herpesviruses: Biology, Therapy, and Immunoprophylaxis. Chapter 54. With assistance of Arvin A, Campadelli-Fiume G, Mocarski E, et al., editors. Cambridge: Cambridge University Press; 2007. Available online at https://www.ncbi.nlm.nih.gov/books/NBK47381/.
Mason, Gavin M.; Poole, Emma; Sissons, J. G. Patrick; Wills, Mark R.; Sinclair, John H. (2012): Human cytomegalovirus latency alters the cellular secretome, inducing cluster of differentiation (CD)4+ T-cell migration and suppression of effector function. In Proceedings of the National Academy of Sciences of the United States of America 109 (36), pp. 14538-14543. DOI: 10.1073/pnas.1204836109.
Mechelli, Rosella; Manzari, Caterina; Policano, Claudia; Annese, Anita; Picardi, Ernesto; Umeton, Renato et al. (2015): Epstein-Barr virus genetic variants are associated with multiple sclerosis. In Neurology 84 (13), pp. 1362-1368. DOI: 10.1212/WNL.0000000000001420.
Middleton P J et. al. (1980): Herpes-Simplex Viral Genome and Senile and Presenile Dementias of Alzheimers and Pick. In Lancet 315 (8176), p. 1038.
Milind, Nikhil; Preuss, Christoph; Haber, Annat; Ananda, Guruprasad; Mukherjee, Shubhabrata; John, Cai et al. (2020): Transcriptomic stratification of late-onset Alzheimer's cases reveals novel genetic modifiers of disease pathology. In PLoS genetics, e1008775. DOI: 10.1371/journal.pgen.1008775.
Miyakawa, T. (1997): Electron microscopy of amyloid fibrils and microvessels. In Annals of the New York Academy of Sciences 826, pp. 25-34. DOI: 10.1111/j.1749-6632.1997.tb48458.x.
Miyani, B.; McCall, C.; Xagoraraki, I. (2020): High abundance of human herpesvirus 8 in wastewater from a large urban area. In Journal of applied microbiology. DOI: 10.1111/jam.14895.
Mocarski, E. S.; Pereira, L.; McCormick, A. L. (1988): Human cytomegalovirus ICP22, the product of the HWLF1 reading frame, is an early nuclear protein that is released from cells. In The Journal of general virology 69 (Pt 10), pp. 2613-2621. DOI: 10.1099/0022-1317-69-10-2613.
Mubangizi, Vincent; Maling, Samuel; Obua, Celestino; Tsai, Alexander C. (2020): Prevalence and correlates of Alzheimer's disease and related dementias in rural Uganda. Cross-sectional, population-based study. In BMC geriatrics 20 (1), p. 48. DOI: 10.1186/s12877-020-1461-z.
Nagel, Maria A.; White, Teresa; Khmeleva, Nelly; Rempel, April; Boyer, Philip J.; Bennett, Jeffrey L. et al. (2015): Analysis of Varicella-Zoster Virus in Temporal Arteries Biopsy Positive and Negative for Giant Cell Arteritis. In JAMA neurology 72 (11), pp. 1281-1287. DOI: 10.1001/jamaneurol.2015.2101.
Nakamura, Akinori; Kaneko, Naoki; Villemagne, Victor L.; Kato, Takashi; Doecke, James; Doré, Vincent et al. (2018): High performance plasma amyloid-β biomarkers for Alzheimer's disease. In Nature 554 (7691), pp. 249-254. DOI: 10.1038/nature25456.
Nath, Avindra (2018): Association of Herpes Viral Infections, Antiherpetic Therapy, and Dementia. Real or Alternative Fact? In Neurotherapeutics: the journal of the American Society for Experimental NeuroTherapeutics 15 (2), pp. 415-416. DOI: 10.1007/s13311-018-0625-4.
Norton, Maria C.; Smith, Ken R.; Ostbye, Truls; Tschanz, JoAnn T.; Corcoran, Chris; Schwartz, Sarah et al. (2010): Greater risk of dementia when spouse has dementia? The Cache County study (58).
Olsson, Jan; Lovheim, Hugo; Honkala, Emma; Karhunen, Pekka J.; Elgh, Fredrik; Kok, Eloise H. (2016): HSV presence in brains of individuals without dementia: the TASTY brain series. In Disease models & mechanisms 9 (11), pp. 1349-1355. DOI: 10.1242/dmm.026674.
Ounanian, Annette; Guilbert, Brigitte; Seigneurin, Jean-Marie (1992): Characteristics of Epstein-Barr virus transformed B cell lines from patients with Alzheimer's disease and age-matched controls. In Mechanisms of ageing and development 63 (1), pp. 105-116. DOI: 10.1016/0047-6374(92)90020-e.
Palmqvist, Sebastian; Janelidze, Shorena; Quiroz, Yakeel T.; Zetterberg, Henrik; Lopera, Francisco; Stomrud, Erik et al. (2020): Discriminative Accuracy of Plasma Phospho-tau217 for Alzheimer Disease vs Other Neurodegenerative Disorders. In JAMA 324 (8), pp. 772-781. DOI: 10.1001/jama.2020.12134.
Panikkar, Archana; Smith, Corey; Hislop, Andrew; Tellam, Nick; Dasari, Vijayendra; Hogquist, Kristin A. et al. (2015): Cytokine-Mediated Loss of Blood Dendritic Cells During Epstein-Barr Virus-Associated Acute Infectious Mononucleosis. Implication for Immune Dysregulation. In The Journal of infectious diseases 212 (12), pp. 1957-1961. DOI: 10.1093/infdis/jiv340.
Parry, Helen M.; Zuo, Jianmin; Frumento, Guido; Mirajkar, Nikhil; Inman, Charlotte; Edwards, Emma et al. (2016): Cytomegalovirus viral load within blood increases markedly in healthy people over the age of 70 years. In Immunity & ageing: I & A 13, p. 1. DOI: 10.1186/s12979-015-0056-6.
Patwardhan, Anand; Bardin, Sabine; Miserey-Lenkei, Stephanie; Larue, Lionel; Goud, Bruno; Raposo, Graga; Delevoye, Cedric (2017): Routing of the RAB6 secretory pathway towards the lysosome related organelle of melanocytes. In Nature communications 8, p. 15835. DOI: 10.1038/ncomms15835.
Pegtel, D. Michiel; Cosmopoulos, Katherine; Thorley-Lawson, David A.; van Eijndhoven, Monique A. J.; Hopmans, Erik S.; Lindenberg, Jelle L. et al. (2010): Functional delivery of viral miRNAs via exosomes. In Proceedings of the National Academy of Sciences of the United States of America 107 (14), pp. 6328-6333. DOI: 10.1073/pnas.0914843107.
Pfeffer, Sébastien; Zavolan, Mihaela; Grässer, Friedrich A.; Chien, Minchen; Russo, James J.; Ju, Jingyue et al. (2004): Identification of virus-encoded microRNAs. In Science (New York, N.Y.) 304 (5671), pp. 734-736. DOI: 10.1126/science.1096781.
Phelan, Elizabeth A.; Borson, Soo; Grothaus, Louis; Balch, Steven; Larson, Eric B. (2012): Association of incident dementia with hospitalizations. In JAMA 307 (2), pp. 165-172. DOI: 10.1001/jama.2011.1964.
Polizzotto, Mark N.; Uldrick, Thomas S.; Wang, Victoria; Aleman, Karen; Wyvill, Kathleen M.; Marshall, Vickie et al. (2013): Human and viral interleukin-6 and other cytokines in Kaposi sarcoma herpesvirus-associated multicentric Castleman disease. In Blood 122 (26), pp. 4189-4198. DOI: 10.1182/blood-2013-08-519959.
Poole, Emma; Sinclair, John (2015): Sleepless latency of human cytomegalovirus. In Medical microbiology and immunology 204 (3), pp. 421-429. DOI: 10.1007/s00430-015-0401-6.
Powers, James F.; Bedri, Shahinaz; Hussein, Shakir; Salomon, Robert N.; Tischler, Arthur S. (2005): High prevalence of herpes simplex virus DNA in temporal arteritis biopsy specimens. In American journal of clinical pathology 123 (2), pp. 261-264. DOI: 10.1309/2996tt2ctltkn0kt.
Pyoria, Lari; Jokinen, Maija; Toppinen, Mari; Salminen, Henri; Vuorinen, Tytti; Hukkanen, Veijo et al. (2020): HERQ-9 Is a New Multiplex PCR for Differentiation and Quantification of All Nine Human Herpesviruses. In mSphere 5 (3). DOI: 10.1128/mSphere.00265-20.
Qu, Lirong; Jenkins, Frank; Triulzi, Darrell J. (2010): Human herpesvirus 8 genomes and seroprevalence in United States blood donors. In Transfusion 50 (5), pp. 1050-1056. DOI: 10.1111/j.1537-2995.2009.02559.x.
Readhead, Ben; Haure-Mirande, Jean-Vianney; Funk, Cory C.; Richards, Matthew A.; Shannon, Paul; Haroutunian, Vahram et al. (2018): Multiscale Analysis of Independent Alzheimer's Cohorts Finds Disruption of Molecular, Genetic, and Clinical Networks by Human Herpesvirus. In Neuron 99 (1), 64-82.e7. DOI: 10.1016/j.neuron.2018.05.023.
Renvoize, E. B.; Hambling, M. H.; Pepper, M. D.; Rajah, S. M. (1979): Possible association of Alzheimer's disease with HLA-BW15 and cytomegalovirus infection. In The Lancet 1 (8128), p. 1238.
Ross, S. A. (2011): Diagnosis of Cytomegalovirus Infections.
Russ, Tom C.; Batty, G. David; Hearnshaw, Gena F.; Fenton, Candida; Starr, John M. (2012): Geographical variation in dementia. Systematic review with meta-analysis. In International journal of epidemiology 41 (4), pp. 1012-1032. DOI: 10.1093/ije/dys103.
Salina, Matteo; Giavazzi, Fabio; Lanfranco, Roberta; Ceccarello, Erica; Sola, Laura; Chiari, Marcella et al. (2015): Multi-spot, label-free immunoassay on reflectionless glass. In Biosensors & bioelectronics 74, pp. 539-545. DOI: 10.1016/j.bios.2015.06.064.
Sänchez-Ponce, Yessica; Varela-Fascinetto, Gustavo; Romo-Vázquez, José Carlos; López-Martinez, Briceida; Sánchez-Huerta, José Luis; Parra-Ortega, Israel et al. (2018): Simultaneous Detection of Beta and Gamma Human Herpesviruses by Multiplex qPCR Reveals Simple Infection and Coinfection Episodes Increasing Risk for Graft Rejection in Solid Organ Transplantation. In Viruses 10 (12). DOI: 10.3390/v10120730.
Schlager, Max A.; Kapitein, Lukas C.; Grigoriev, Ilya; Burzynski, Grzegorz M.; Wulf, Phebe S.; Keijzer, Nanda et al. (2010): Pericentrosomal targeting of Rab6 secretory vesicles by Bicaudal-D-related protein 1 (BICDR-1) regulates neuritogenesis. In The EMBO journal 29 (10), pp. 1637-1651. DOI: 10.1038/emboj.2010.51.
Sedlackova, Lenka; Perkins, Keith D.; Lengyel, Joy; Strain, Anna K.; van Santen, Vicky L.; Rice, Stephen A. (2008): Herpes Simplex Virus Type 1 ICP27 Regulates Expression of a Variant, Secreted Form of Glycoprotein C by an Intron Retention Mechanism. In J. Virol. 82 (15), pp. 7443-7455. DOI: 10.1128/JVI.00388-08.
Shipley, Suzanne J.; Parkin, Edward T.; Itzhaki, Ruth F.; Dobson, Curtis B. (2005): Herpes simplex virus interferes with amyloid precursor protein processing. In BMC microbiology 5, p. 48. DOI: 10.1186/1471-2180-5-48.
Steele, Natasha Z. R.; Carr, Jessie S.; Bonham, Luke W.; Geier, Ethan G.; Damotte, Vincent; Miller, Zachary A. et al. (2017): Fine-mapping of the human leukocyte antigen locus as a risk factor for Alzheimer disease. A case-control study. In PLoS medicine 14 (3), e1002272. DOI: 10.1371/journal.pmed.1002272.
Steiner, I.; Mador, N.; Reibstein, I.; Spivack, J. G.; Fraser, N. W. (1994): Herpes simplex virus type 1 gene expression and reactivation of latent infection in the central nervous system. In Neuropathology and applied neurobiology 20 (3), pp. 253-260.
Stewart, Michael W.; Bolling, James P.; Mendez, Julio C. (2005): Cytomegalovirus retinitis in an immunocompetent patient. In Archives of ophthalmology (Chicago, Ill.: 1960) 123 (4), pp. 572-574. DOI: 10.1001/archopht.123.4.572.
Stowe, Raymond P.; Kozlova, Elena V.; Yetman, Deborah L.; Walling, Dennis M.; Goodwin, James S.; Glaser, Ronald (2007): Chronic herpesvirus reactivation occurs in aging. In Experimental gerontology 42 (6), pp. 563-570. DOI: 10.1016/j.exger.2007.01.005.
Taga, H.; Taga, K.; Wang, F.; Chretien, J.; Tosato, G. (1995): Human and viral interleukin-10 in acute Epstein-Barr virus-induced infectious mononucleosis. In The Journal of infectious diseases 171 (5), pp. 1347-1350. DOI: 10.1093/infdis/171.5.1347.
Tang, et. al. (1997): Molecular Evidence and Clinical Significance of Herpesvirus Coinfection in the Central Nervous System. In Journal of Clinical Microbiology 35 (11), pp. 2869-2872.
Teow, Sin-Yeang; Liew, Kitson; Khoo, Alan Soo-Beng; Peh, Suat-Cheng (2017): Pathogenic Role of Exosomes in Epstein-Barr Virus (EBV)-Associated Cancers. In International journal of biological sciences 13 (10), pp. 1276-1286. DOI: 10.7150/ijbs.19531.
Thambisetty, Madhav; Simmons, Andrew; Hye, Abdul; Campbell, James; Westman, Eric; Zhang, Yi et al. (2011): Plasma biomarkers of brain atrophy in Alzheimer's disease. In PloS one 6 (12), e28527. DOI: 10.1371/journal.pone.0028527. Theil, Diethilde; Horn, Anja K. E.; Derfuss, Tobias; Strupp, Michael; Arbusow, Viktor; Brandt, Thomas (2004): Prevalence and distribution of HSV-1, VZV, and HHV-6 in human cranial nerve nuclei III, IV, VI, VII, and XII. In Journal of medical virology 74 (1), pp. 102-106. DOI: 10.1002/jmv.20152.
Timens, W.; Boes, A.; Vos, H.; Poppema, S. (1991): Tissue distribution of the C3d/EBV-receptor. CD21 monoclonal antibodies reactive with a variety of epithelial cells, medullary thymocytes, and peripheral T-cells. In Histochemistry 95 (6), pp. 605-611. DOI: 10.1007/BF00266748.
Toczyski, D. P.; Matera, A. G.; Ward, D. C.; Steitz, J. A. (1994): The Epstein-Barr virus (EBV) small RNA EBER1 binds and relocalizes ribosomal protein L22 in EBV-infected human B lymphocytes. In Proceedings of the National Academy of Sciences 91 (8), pp. 3463-3467. DOI: 10.1073/pnas.91.8.3463.
Top; Rainy, Nir; Zayoud, Morad; Kloog, Yoel; Rechavi, Oded; Goldstein, Itamar (2016): Viral oncomiR spreading between B and T cells is employed by Kaposi's sarcoma herpesvirus to induce non-cell-autonomous target gene regulation. In Oncotarget 7 (27), pp. 41870-41884. DOI: 10.18632/oncotarget.9627.
Tsai, Ming-Chieh; Cheng, Wan-Ling; Sheu, Jau-Jiuan; Huang, Chung-Chien; Shia, Ben-Chang; Kao, Li-Ting; Lin, Herng-Ching (2017): Increased risk of dementia following herpes zoster ophthalmicus. In PloS one 12 (11), e0188490. DOI: 10.1371/journal.pone.0188490.
Tso, For Yue; Sawyer, Ashley; Kwon, Eun Hee; Mudenda, Victor; Langford, Dianne; Zhou, You et al. (2017): Kaposi's Sarcoma-Associated Herpesvirus Infection of Neurons in HIV-Positive Patients. In The Journal of infectious diseases 215 (12), pp. 1898-1907. DOI: 10.1093/infdis/jiw545.
Uematsu, Miho; Nakamura, Ayako; Ebashi, Momoko; Hirokawa, Katsuiku; Takahashi, Ryosuke; Uchihara, Toshiki (2018): Brainstem tau pathology in Alzheimer's disease is characterized by increase of three repeat tau and independent of amyloid β. In Acta neuropathologica communications 6 (1), p. 1. DOI: 10.1186/s40478-017-0501-1.
Uldrick, Thomas S.; Wang, Victoria; O'Mahony, Deirdre; Aleman, Karen; Wyvill, Kathleen M.; Marshall, Vickie et al. (2010): An interleukin-6-related systemic inflammatory syndrome in patients co-infected with Kaposi sarcoma-associated herpesvirus and HIV but without Multicentric Castleman disease. In Clinical infectious diseases: an official publication of the Infectious Diseases Society of America 51 (3), pp. 350-358. DOI: 10.1086/654798.
Vanheusden, Marjan; Broux, Bieke; Welten, Suzanne P. M.; Peeters, Liesbet M.; Panagioti, Eleni; van Wijmeersch, Bart et al. (2017): Cytomegalovirus infection exacerbates autoimmune mediated neuroinflammation. In Sci Rep 7 (1), p. 134. DOI: 10.1038/s41598-017-00645-3.
Warren-Gash, Charlotte; Forbes, Harriet J.; Williamson, Elizabeth; Breuer, Judith; Hayward, Andrew C.; Mavrodaris, Angelique et al. (2019): Human herpesvirus infections and dementia or mild cognitive impairment. A systematic review and meta-analysis. In Scientific reports 9 (1), p. 4743. DOI: 10.1038/s41598-019-41218-w.
Wee, Sheena; Alli-Shaik, Asfa; Kek, Relus; Swa, Hannah L. F.; Tien, Wei-Ping; Lim, Vanessa W. et al. (2019): Multiplex targeted mass spectrometry assay for one-shot flavivirus diagnosis. In Proceedings of the National Academy of Sciences of the United States of America 116 (14), pp. 6754-6759. DOI: 10.1073/pnas.1817867116.
Westman, Gabriel; Berglund, David; Widen, Johan; Ingelsson, Martin; Korsgren, Olle; Lannfelt, Lars et al. (2014): Increased inflammatory response in cytomegalovirus seropositive patients with Alzheimer's disease. In PloS one 9 (5), e96779. DOI: 10.1371/journal.pone.0096779.
Wu, Hongling; Li, Ting; Zeng, Musheng; Peng, Tao (2012): Herpes simplex virus type 1 infection activates the Epstein-Barr virus replicative cycle via a CREB-dependent mechanism. In Cellular microbiology, pp. 546-559. DOI: 10.1111/j.1462-5822.2011.01740.x.
Yogev, Ohad; Henderson, Stephen; Hayes, Matthew John; Marelli, Sara Sofia; Ofir-Birin, Yifat; Regev-Rudzki, Neta et al. (2017): Herpesviruses shape tumour microenvironment through exosomal transfer of viral microRNAs. In PLoS pathogens 13 (8), e1006524. DOI: 10.1371/journal.ppat.1006524.
Young, R. F.; Perryman, K. M. (1984): Pathways for orofacial pain sensation in the trigeminal brain-stem nuclear complex of the Macaque monkey. In Journal of neurosurgery 61 (3), pp. 563-568. DOI: 10.3171/jns.1984.61.3.0563.
Young, Vivian (2015): Detection of HCMV viral IL-10 (vIL-10) in healthy blood donors. Master's Thesis. University of San Francisco, San Francisco. USFCA. Available online at https:/repository.usfca.edu/cgi/viewcontent.cgi?article=1182&context=thes, checked on 11/27/2020.
Young, Vivian P.; Mariano, Margarette C.; Tu, Carolyn C.; Allaire, Kathryn M.; Avdic, Selmir; Slobedman, Barry; Spencer, Juliet V. (2017): Modulation of the Host Environment by Human Cytomegalovirus with Viral Interleukin 10 in Peripheral Blood. In The Journal of infectious diseases 215 (6), pp. 874-882. DOI: 10.1093/infdis/jix043.
Zanchetta, Giuliano; Carzaniga, Thomas; Vanjur, Luka; Casiraghi, Luca; Tagliabue, Giovanni; Morasso, Carlo et al. (2020): Design and Optimization of a Rapid, Multiplex miRNA Assay without Washing Steps. In Proceedings 60 (1), p. 32. DOI: 10.3390/IECB2020-07040.
Zhang, Guoan; Ueberheide, Beatrix M.; Waldemarson, Sofia; Myung, Sunnie; Molloy, Kelly; Eriksson, Jan et al. (2010): Protein quantitation using mass spectrometry. In Methods in molecular biology (Cifton, N.J.) 673, pp. 211-222. DOI: 10.1007/978-1-60761-842-3_13.
Zhang, Ming; Atherton, Sally S. (2002): Apoptosis in the retina during MCMV retinitis in immunosuppressed BALB/c mice. In Journal of Clinical Virology 25, pp. 137-147. DOI: 10.1016/s1386-6532(02)00102-6.
Zhao, Zhuoxian; Rocha, Natalia P.; Salem, Haitham; Diniz, Breno S.; Teixeira, Antonio L. (2018): The association between systemic lupus erythematosus and dementia A meta-analysis. In Dementia & neuropsychologia 12 (2), pp. 143-151. DOI: 10.1590/1980-57642018dn12-020006.

1. A method of diagnosing risk of herpesvirus-mediated Alzheimer's disease in a human subject comprising,
(a) isolating exosomes from a serum sample obtained from a subject by contacting exosomes with an isolation reagent, and incubating serum to precipitate the exosomes, and then centrifuging to pellet the exosomes; (b) releasing the proteins from the exosomes by organic extraction and immobilization on a glass fiber filter, (c) detecting herpesvirus-encoded proteins by contacting the exosome proteins with a plurality of detection reagents

2. [symbol] wherein the plurality of detection reagents comprises at least 50 detection reagents; (d) measuring the amount of the herpesvirus-encoded protein detected in the blood sample by each detection reagent; (e) comparing the amount of herpesvirus-encoded protein detected in the exosome sample by each detection reagent to a predetermined background control amount; wherein the detection over a background control value confers a diagnosis for risk of herpesvirus-mediated Alzheimer's disease.(Currently amended) A method according to claim 1, wherein the human cytomegalovirus encoded protein detected is selected from the group consisting of pp52 (SEQ ID NO 1), IE1 (SEQ ID NO 2), IE2 (SEQ ID NO 3), pp150 (SEQ ID NO 4), UL21.5 (SEQ ID NO 5), UL7 (SEQ ID NO 6), UL128 (SEQ ID NO 7), vCXCL-1 (SEQ ID NO 8), vCXCL-2 (SEQ ID NO 9), early nuclear protein HWLF1 (SEQ ID NO 12), US34 (SEQ ID NO 13), or UL13 (SEQ ID NO 14).

3. A method according to claim 1, wherein the secreted human herpesvirus 6A encoded protein detected is selected from the group consisting of pp100 (SEQ ID NO 15), IE 1 (SEQ ID NO 18), IE2 (SEQ ID NO 19), PPS (SEQ ID NO 20), glycoprotein B (SEQ ID NO 21), or DR1/DR2 (SEQ ID NO 22).

4. A method according to claim 1, wherein the secreted human herpesvirus 7 encoded protein detected is selected from the group consisting of pp100 (SEQ ID NO 16), IE1 (SEQ ID NO 25), IE2 (SEQ ID NO 26), PPS (SEQ ID NO 27), glycoprotein B (SEQ ID NO 28), or DR1/DR2 (SEQ ID NO 29).

5. A method according to claim 1, wherein the secreted Epstein-Barr virus encoded protein detected is selected from the group consisting of, BHRF1-1 (SEQ ID NO 33), BHRF1-2 (SEQ ID NO 34), BHRF1-3 (SEQ ID NO 35), LMP1 (SEQ ID NO 51), LMP2A (SEQ ID NO 52), or ebvIL-10 (SEQ ID NO 53).

6. A method according to claim 1, wherein the secreted human simplex virus type 1 encoded protein detected is selected from the group consisting of VP22 (SEQ ID NO 54), glycoprotein B (SEQ ID NO 55), glycoprotein C (SEQ ID NO 56), or UL56 (SEQ ID NO 57).

7. A method according to claim 1, wherein the secreted varicella-zoster virus encoded protein detected is selected from the group consisting of glycoprotein B (SEQ ID NO 61), glycoprotein E (SEQ ID NO 62), glycoprotein I (SEQ ID NO 63), glycoprotein C (SEQ ID NO 64), glycoprotein H (SEQ ID NO 65), or glycoprotein L (SEQ ID NO 66).

8. A method according to claim 1, wherein the secreted Kaposi's sarcoma virus encoded protein detected is selected from the group consisting of vIL-6 (SEQ ID NO 67), ORF 8.1 (SEQ ID NO 68), ORF 4 (SEQ ID NO 69).

9. (canceled)

10. A method according to claim 1, wherein secreted herpesvirus-encoded proteins from the nine human herpesvirus types are screened in a human subject to identify the total number of herpesvirus types infecting a subject, wherein detection of proteins over a background control indicates the presence of infection, and wherein infection with multiple herpesvirus types prescribes monitoring a subject's herpesvirus-encoded protein levels.

11. (canceled)

12. A diagnostic kit for diagnosing the risk of human herpesvirus-mediated Alzheimer's disease by measuring human herpesvirus encoded proteins secreted from cells in a blood sample obtained from a human subject, the kit comprising: (a) an antibody or aptamer which specifically binds to a secreted human herpesvirus-encoded protein, (b) a solid matrix or microplate well to which a capture antibody or aptamer is bound, (c) a secondary antibody or aptamer permitting the selective formation of a complex between a human herpesvirus protein and two antibodies or aptamers, or one antibody and one aptamer, (d) recombinant herpesvirus protein standard(e) reagents for detection.

13. (canceled)

14. A kit according to claim 12, wherein an antibody or aptamer binds to a secreted human cytomegalovirus-encoded protein, the protein selected from the group consisting of pp52 (SEQ ID NO 1), IE1 (SEQ ID NO 2), IE2 (SEQ ID NO 3), pp150 (SEQ ID NO 4), pUL21.5 (SEQ ID NO 5), UL7 (SEQ ID NO 6), UL128 (SEQ ID NO 7), vCXCL-1 (SEQ ID NO 8), vCXCL-2 (SEQ ID NO 9), vIL-10 (SEQ ID NO 10), and protein LAvIL-10 (SEQ ID NO 11), early nuclear protein HWLF1 (SEQ ID NO 12), US34 (SEQ ID NO 13), or UL13 (SEQ ID NO 14)

15. A kit according to claim 12, wherein the antibody or aptamer binds to a secreted human herpesvirus 6A-encodedprotein, the proteins are selected from the group consisting of pp100 (SEQ ID NO 15), IE1 (SEQ ID NO 18), IE2 (SEQ ID NO 19), PPS (SEQ ID NO 20), glycoprotein B (SEQ ID NO 21) or DR1/DR2 (SEQ ID NO 22).

16. A kit according to claim 12, wherein the antibody or aptamer binds to a secreted human herpesvirus 7-encodedprotein, the protein selected from the group consisting of pp100 (SEQ ID NO 16), IE1 (SEQ ID NO 25), IE2 (SEQ ID NO 26), PPS (SEQ ID NO 27), glycoprotein B (SEQ ID NO 28) or DR1/DR2 (SEQ ID NO 29).

17. A kit according to claim 12, wherein the antibody or aptamer binds to a secreted Epstein-Barr virus protein, the protein selected from the group consisting of, LMP1 (SEQ ID NO 51), LMP2A (SEQ ID NO 52), or protein ebvIL-10 (SEQ ID NO 53).

18. A kit according to claim 12, wherein the antibody or aptamer binds to a secreted human simplex virus type 1protein, the protein selected from the group consisting of VP22 protein (SEQ ID NO 54), glycoprotein B (SEQ ID NO 55), glycoprotein C (SEQ ID NO 56), or UL56 (SEQ ID NO 57).

19. A kit according to claim 12, wherein the antibody or aptamer binds to a secreted varicella-zoster virusprotein, the protein selected from the group consisting of glycoprotein B (SEQ ID NO 61), glycoprotein E (SEQ ID NO 62), glycoprotein I (SEQ ID NO 63), glycoprotein C (SEQ ID NO 64), glycoprotein H (SEQ ID NO 65), or glycoprotein L (SEQ ID NO 66).

20. (canceled)

21. A method according to claim 1, wherein the human simplex virus type 2 encoded protein detected selected from the group consisting of glycoprotein B (SEQ ID NO 58), N-terminal glycoprotein G (SEQ ID NO 59), or the peptide gG-2p20 (SEQ ID NO 60).

22. A method of detecting diagnosing risk of herpesvirus-mediated Alzheimer's disease neurodegeneration in a human subject comprising, (a) isolating exosomes from a serum sample obtained from a subject by contacting exosomes with an isolation reagent, and incubating serum to precipitate the exosomes, and then centrifuging to pellet the exosomes; (b) releasing the proteins from the exosomes by organic extraction and immobilization on a glass fiber filter, (c) detecting herpesvirus-encoded proteins by cleaning with trypsin digestion and isoelectric fractionation and processing by liquid chromatography and mass spectrometry and analyzing the data (e) comparing the amount of herpesvirus-encoded protein detected in the exosome sample by each detection reagent to a predetermined background control amount; wherein the detection over a background control value confers a diagnosis for risk of herpesvirus-mediated Alzheimer's disease.
